(19) United States
(12) Patent Application Publication
(10) Pub. No.: US 2025/0082832 A1
(43) Pub. Date: Mar. 13, 2025

(54) INSERTION CATHETER FOR A CIRCULATORY SUPPORT CATHETER

(71) Applicant: EDWARDS LIFESCIENCES CORPORATION, Irvine, CA (US)

(72) Inventors: Hans Christof, Unterensingen (DE); Leon Wenning, Stuttgart (DE); Kenneth M. Martin, Woodside, CA (US); Inga Schellenberg, Stuttgart (DE); Hardy Baumbach, Suttgart (DE); Thomas Friedrich, Stuttgart (DE); Mario Heintze, Stuttgart (DE); Hien T. Nguyen, Aliso Viejo, CA (US); Daniel James Murray, Orange, CA (US); Michael G. Valdez, Riverside, CA (US); Yair A. Neumann, Moshav Sede Varburg (IL); Anne Bernadette Aragon Alcasid, Costa Mesa, CA (US); Amir Davidesko, Binyamina Givat Ada (IL); Steven Park, Santa Ana, CA (US); Sofia Rozenberg, Tel-Aviv (IL); Linda Thai, Mission Viejo, CA (US); Cristobal R. Hernandez, Santa Ana, CA (US); Omar Fawzi Azanki, Coto de Caza, CA (US)

(21) Appl. No.: 18/957,242

(22) Filed: Nov. 22, 2024

Related U.S. Application Data
(63) Continuation of application No. PCT/US2023/023400, filed on May 24, 2023.
(60) Provisional application No. 63/345,182, filed on May 24, 2022.

(51) Int. Cl. A61M 1/36

(52) U.S. Cl. CPC A61M 1/36

(57) ABSTRACT

An insertion catheter for a circulatory support catheter having a circulatory support device carried by an elongate flexible catheter shaft. The insertion catheter comprises a tubular body with a distal portion configured to axially movably receive the circulatory support device. A diameter of the distal portion is greater than a diameter of a mid portion of the tubular body. The insertion catheter may be inserted into an introducer sheath for delivery of the MCS device. A distal end of the insertion catheter may be located distally of a distal end of the introducer sheath and distally of the arterial bifurcation. The distal portion of the insertion catheter containing the MCS device may be located distally of the bifurcation, with a distal end of the MCS device slightly offset from the distal end of the tubular body.

### INCORPORATION BY REFERENCE TO ANY PRIORITY APPLICATIONS

This application is a continuation of International Patent Application No. PCT/US2023/023400, filed May 24, 2023, which claims the benefit of U.S. Provisional Application No. 63/345,182, filed on May 24, 2022, the complete disclosures of which are hereby incorporated by reference in their entireties.

### BACKGROUND

### Technical Field

The present disclosure is directed generally to mechanical cardiovascular support systems used in the medical field to assist the movement of blood. In particular, the present disclosure is directed to improved insertion catheters, introducer sheaths, and various components thereof for optimized delivery of mechanical circulatory support devices into the heart.

### Description of the Related Art

Mechanical circulatory support systems may be used to assist with pumping blood during various medical procedures and/or as therapy for certain cardiac conditions. For example, cardiogenic shock (CS) is a common cause of mortality, and management remains challenging despite advances in therapeutic options. CS is caused by severe impairment of myocardial performance that results in diminished cardiac output, end-organ hypoperfusion, and hypoxia. Clinically this presents as hypotension refractory to volume resuscitation with features of end-organ hypoperfusion requiring immediate pharmacological or mechanical intervention. Acute myocardial infarction (MI) accounts for over about 80% of patients in CS.

As further example, percutaneous coronary intervention (PCI) is a non-surgical procedure to revascularize stenotic coronary arteries. PCI includes a variety of techniques, e.g. balloon angioplasty, stent implantation, rotablation and lithotripsy. A PCI is considered high risk if either the patient has relevant comorbidities (e.g. frailty or advanced age), the PCI per se is very complex (e.g. bifurcation or total occlusions) or hemodynamic status is challenging (e.g. impaired ventricular function).

Miniature, catheter-based intracardiac blood pumps have been developed for percutaneous insertion into a patient's body as an acute therapy for CS and for temporary assistance during PCI. However, existing solutions for pumps have various performance deficiencies such as, for example, inadequate blood flow, the requirement for ongoing motor purging within the pump, undesirably high hemolysis, and inadequate sensing of hemodynamic parameters. Thus, there remains a need for mechanical circulatory support systems with features that overcome these and other drawbacks as well as improved ways to introduce such systems into the body.

### SUMMARY

The embodiments disclosed herein each have several aspects no single one of which is solely responsible for the disclosure's desirable attributes. Without limiting the scope of this disclosure, its more prominent features will now be briefly discussed. After considering this discussion, and particularly after reading the section entitled “Detailed Description,” one will understand how the features of the embodiments described herein provide advantages over existing systems, devices and methods for circulatory support systems.

The following disclosure describes non-limiting examples of some embodiments. For instance, other embodiments of the disclosed systems and methods may or may not include the features described herein. Moreover, disclosed advantages and benefits can apply only to certain embodiments and should not be used to limit the disclosure.

In some embodiments, disclosed herein is an insertion catheter for a circulatory support catheter having a circulatory support device carried by an elongate flexible catheter shaft, the insertion catheter comprising: a tubular body having a proximal end, a distal end, a longitudinal axis extending between the proximal and distal ends, a proximal portion adjacent the proximal end, a distal portion adjacent the distal end, and a mid portion between the proximal portion and the distal portion, wherein the distal portion of the tubular body is configured to axially movably receive the circulatory support device, and wherein a diameter of the distal portion is greater than a diameter of the mid portion.

In the above insertion catheter or in other embodiments as described herein, one or more of the following features may also be provided. In some embodiments, the insertion catheter further comprises a hub having a proximal end and a distal end, wherein the distal end of the hub fluidly connects with the proximal end of the tubular body. In some embodiments, the tubular body further comprises a proximal transition section between the proximal portion and the mid portion and a distal transition section between the distal portion and the mid portion. In some embodiments, the distal portion and the distal transition section are configured to receive the circulatory support device and the mid portion, proximal transition section, proximal portion, and hub are configured to receive the elongate flexible catheter shaft. In some embodiments, the distal portion, mid portion, and proximal portion are concentric about the longitudinal axis. In some embodiments, the mid portion comprises a smaller outer diameter than an outer diameter of the proximal portion and an outer diameter of the distal portion. In some embodiments, the distal portion comprises a larger inner diameter than an inner diameter of the mid portion and the proximal portion. In some embodiments, the tubular body is configured so that the proximal portion remains outside the body of a patient when the insertion catheter is in use. In some embodiments, the tubular body is configured so that the mid portion extends from outside the body of a patient through an arteriotomy of a femoral artery, through the femoral artery, through a bifurcation of the femoral artery where it joins an aorta, and into the aorta when the insertion catheter is in use. In some embodiments, the tubular body is configured so that the distal portion is positioned within an aorta of a patient when the insertion catheter is in use. In some embodiments, the distal end of the tubular body is configured to extend past a distal end of an introducer sheath when the insertion catheter is fully docked with the introducer sheath when in use. In some embodiments, the tubular body of the insertion catheter has sufficient collapse resistance to maintain patency when passed through one or more hemostatic valves of the introducer sheath. In some embodiments, the distal end of the hub comprises one or more features for preventing rotational and/or axial movement of the insertion catheter when docked with the introducer sheath. In some embodiments, the insertion catheter comprises a tube with a valve in fluid communication with an inner lumen of the tubular body of the insertion catheter configured for flushing with saline. In some embodiments, the insertion catheter comprises a hemostatic valve. In some embodiments, the insertion catheter comprises a plug disposed at the proximal end of the hub configured to connect to a sterile shield sleeve. In some embodiments, the elongate flexible catheter shaft comprises a visual marker spaced proximally from the circulatory support device such that visibility of the visual marker on a proximal side of the hub indicates the circulatory support device is located within the tubular body of the insertion catheter. In some embodiments, the circulatory support device comprises a tubular housing, a motor, an impeller configured to be rotated by the motor, a first guidewire port on a distal end of the tubular housing, a second guidewire port on a sidewall of the tubular housing distal to the impeller, and a third guidewire port on a proximal side of the impeller, and wherein the distal end of the tubular body of the insertion catheter detachably connects to a guidewire aid configured to facilitate entry of a guidewire through the first guidewire port. In some embodiments, the tubular body of the insertion catheter is configured to receive the circulatory support device with a removable guidewire guide tube, wherein the removable guidewire guide tube enters the first guidewire port on the distal end of the tubular housing, exits the tubular housing via the second guidewire port on the sidewall of the tubular housing distal to the impeller, reenters the tubular housing via the third guidewire port on the proximal side of the impeller, and extends proximally into the catheter shaft. In some embodiments, the hub comprises one or more features for preventing axial and optionally rotational movement of the circulatory support catheter. In some embodiments, the hub of the insertion catheter comprises a locking mechanism, the locking mechanism comprising a recess configured to accept a locking pad configured to releasably lock with the circulatory support catheter. In some embodiments, the hub of the insertion catheter comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the circulatory support catheter. In some embodiments, inward compression of the tabs of the locking mechanism compresses the locking pad against the circulatory support catheter. In some embodiments, the locking pad is configured to releasably lock with the catheter shaft of the circulatory support catheter. In some embodiments, the tubular body of the insertion catheter has a length within a range of from about 275 mm to about 675 mm and an inside diameter within a range of from about 1.5 mm to about 6 mm. In some embodiments, the distal portion of the tubular body has a length within a range of from about 75 mm to about 140 mm and an inside diameter within a range of from about 3.5 mm to about 6 mm, wherein the proximal portion of the tubular body has a length within a range of from about 100 mm to about 165 mm and an inside diameter within a range of from about 2.0 mm to about 4.5 mm, and wherein the mid portion of the tubular body has a length within a range of from about 150 mm to about 250 mm and an inside diameter within a range of from about 1.5 mm to about 4.5 mm. In some embodiments, the tubular portion comprises a braided wire. In some embodiments, the distal end of the tubular body comprises a PET band configured to hold ends of the braided wire. In some embodiments, the distal end of the tubular body comprises a radio opaque marker. In some embodiments, the proximal portion of the tubular body is configured to transmit longitudinal force without kinking. In some embodiments, the proximal portion of the tubular body is more stiff than the mid portion and the distal portion. In some embodiments, the proximal portion of the tubular body comprises a reinforced double-braided wire. In some embodiments, at least the distal portion of the tubular body is coated with a hydrophilic coating configured to reduce friction on an exterior surface thereof.

In some embodiments, disclosed herein is a mechanical circulatory support system, comprising: a circulatory support catheter, comprising a circulatory support device carried by an elongate flexible catheter shaft, the circulatory support device comprising a tubular housing, a motor, and an impeller configured to be rotated by the motor; and an insertion catheter comprising a tubular body having a proximal end, a distal end, a longitudinal axis extending between the proximal and distal ends, a proximal portion adjacent the proximal end, a distal portion adjacent the distal end, and a mid portion between the proximal portion and the distal portion, wherein the distal portion of the tubular body is configured to axially movably receive the circulatory support device, and wherein a diameter of the distal portion is greater than a diameter of the mid portion.

In the above system or in other embodiments as described herein, one or more of the following features may also be provided. In some embodiments, the impeller is configured to be rotated by the motor via a shaft. In some embodiments, the impeller is configured to be rotated by the motor via a magnetic coupling. In some embodiments, the system does not require purging. In some embodiments, the insertion catheter further comprises a hub having a proximal end and a distal end, wherein the distal end of the hub fluidly connects with the proximal end of the tubular body. In some embodiments, the tubular body further comprises a proximal transition section between the proximal portion and the mid portion and a distal transition section between the distal portion and the mid portion. In some embodiments, the distal portion and the distal transition section are configured to receive the circulatory support device and the mid portion, proximal transition section, proximal portion, and hub are configured to receive the elongate flexible catheter shaft. In some embodiments, the distal portion, mid portion, and proximal portion are concentric about the longitudinal axis. In some embodiments, the mid portion comprises a smaller outer diameter than an outer diameter of the proximal portion and an outer diameter of the distal portion. In some embodiments, the distal portion comprises a larger inner diameter than an inner diameter of the mid portion and the proximal portion. In some embodiments, the tubular body is configured so that the proximal portion remains outside the body of a patient when the insertion catheter is in use. In some embodiments, the tubular body is configured so that the mid portion extends from outside the body of a patient through an arteriotomy of a femoral artery, through the femoral artery, through a bifurcation of the femoral artery where it joins an aorta, and into the aorta when the insertion catheter is in use. In some embodiments, the tubular body is configured so that the distal portion is positioned within an aorta of a patient when the insertion catheter is in use. In some embodiments, the distal end of the tubular body is configured to extend past a distal end of an introducer sheath when the insertion catheter is fully docked with the introducer sheath when in use. In some embodiments, the distal end of the hub comprises one or more features for preventing rotational and/or axial movement of the insertion catheter when docked with the introducer sheath. In some embodiments, the insertion catheter comprises a tube with a valve in fluid communication with an inner lumen of the tubular body of the insertion catheter configured for flushing with saline. In some embodiments, the insertion catheter comprises a hemostatic valve. In some embodiments, the insertion catheter comprises a plug disposed at the proximal end of the hub configured to connect to a sterile shield sleeve. In some embodiments, the elongate flexible catheter shaft comprises a visual marker spaced proximally from the circulatory support device such that visibility of the visual marker on a proximal side of the hub indicates the circulatory support device is located within the tubular body of the insertion catheter. In some embodiments, the circulatory support device comprises a tubular housing, a motor, an impeller configured to be rotated by the motor, a first guidewire port on a distal end of the tubular housing, a second guidewire port on a sidewall of the tubular housing distal to the impeller, and a third guidewire port on a proximal side of the impeller, and wherein the distal end of the tubular body of the insertion catheter detachably connects to a guidewire aid configured to facilitate entry of a guidewire through the first guidewire port. In some embodiments, the tubular body of the insertion catheter is configured to receive the circulatory support device with a removable guidewire guide tube, wherein the removable guidewire guide tube enters the first guidewire port on the distal end of the tubular housing, exits the tubular housing via the second guidewire port on the sidewall of the tubular housing distal to the impeller, reenters the tubular housing via the third guidewire port on the proximal side of the impeller, and extends proximally into the catheter shaft. In some embodiments, the hub comprises one or more features for preventing axial and optionally rotational movement of the circulatory support catheter. In some embodiments, the hub of the insertion catheter comprises a locking mechanism, the locking mechanism comprising a recess configured to accept a locking pad configured to releasably lock with the circulatory support catheter. In some embodiments, the hub of the insertion catheter comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the circulatory support catheter. In some embodiments, inward compression of the tabs of the locking mechanism compresses the locking pad against the circulatory support catheter. In some embodiments, the locking pad is configured to releasably lock with the catheter shaft of the circulatory support catheter. In some embodiments, the tubular body of the insertion catheter has a length within a range of from about 275 mm to about 675 mm and an inside diameter within a range of from about 1.5 mm to about 6 mm. In some embodiments, the distal portion of the tubular body has a length within a range of from about 75 mm to about 140 mm and an inside diameter within a range of from about 3.5 mm to about 6 mm, wherein the proximal portion of the tubular body has a length within a range of from about 100 mm to about 165 mm and an inside diameter within a range of from about 2.0 mm to about 4.5 mm, and wherein the mid portion of the tubular body has a length within a range of from about 150 mm to about 250 mm and an inside diameter within a range of from about 1.5 mm to about 4.5 mm. In some embodiments, the tubular portion comprises a braided wire. In some embodiments, the distal end of the tubular body comprises a PET band configured to hold ends of the braided wire. In some embodiments, the distal end of the tubular body comprises a radio opaque marker. In some embodiments, the proximal portion of the tubular body is configured to transmit longitudinal force without kinking. In some embodiments, the proximal portion of the tubular body is more stiff than the mid portion and the distal portion. In some embodiments, the proximal portion of the tubular body comprises a reinforced double-braided wire. In some embodiments, at least the distal portion of the tubular body is coated with a hydrophilic coating configured to reduce friction on an exterior surface thereof.

In some embodiments, disclosed herein is a method of using an insertion catheter with a circulatory support catheter having a circulatory support device carried by an elongate flexible catheter shaft, the method comprising: inserting a distal end of the insertion catheter through a proximal end of an introducer sheath that has been advanced into an artery of a patient, and advancing the insertion catheter through the introducer sheath and any hemostatic valves of the introducer sheath until the distal end of the insertion catheter extends past a distal end of the introducer sheath, wherein the insertion catheter comprises a tubular body having a distal portion adjacent the distal end configured to axially movably receive the circulatory support device, and wherein the insertion catheter is configured to protect the circulatory support device as it is advanced through the introducer sheath and any hemostatic valves of the introducer sheath.

In the above method or in other embodiments as described herein, one or more of the following features may also be provided. In some embodiments, the insertion catheter further comprises a hub fluidically connected to a proximal end of the tubular body, the hub configured to lock with the introducer sheath and prevent axial and/or rotational movement of the insertion catheter relative to the introducer sheath when the insertion catheter is fully advanced through the introducer sheath. In some embodiments, the tubular body further comprises a proximal portion adjacent its proximal end and a mid portion between the proximal portion and the distal portion. In some embodiments, the tubular body further comprises a proximal transition section between the proximal portion and the mid portion and a distal transition section between the distal portion and the mid portion. In some embodiments, the distal portion and the distal transition section are configured to receive the circulatory support device and the mid portion, proximal transition section, proximal portion, and hub are configured to receive the elongate flexible catheter shaft. In some embodiments, the tubular body is configured so that the proximal portion remains outside the body of a patient when the insertion catheter is fully advanced through the introducer sheath. In some embodiments, the tubular body is configured so that the distal portion is positioned within an aorta of a patient when the insertion catheter is fully advanced through the introducer sheath. In some embodiments, the tubular body is configured so that the mid portion extends from outside the body of a patient through an arteriotomy of a femoral artery, through the femoral artery, through a bifurcation of the femoral artery where it joins an aorta, and into the aorta when the insertion catheter is fully advanced through the introducer sheath. In some embodiments, the method further comprises advancing the circulatory support catheter through the insertion catheter until its target therapeutic location is reached within a patient. In some embodiments, the hub of the insertion catheter comprises a locking mechanism, and the method further comprises locking the axial location of the circulatory support catheter relative to the insertion catheter. In some embodiments, the locking mechanism comprising a recess configured to accept a locking pad configured to releasably lock with the circulatory support catheter. In some embodiments, the hub of the insertion catheter comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the circulatory support catheter. In some embodiments, inward compression of the tabs of the locking mechanism compresses the locking pad against the circulatory support catheter. In some embodiments, the locking pad is configured to releasably lock with the catheter shaft of the circulatory support catheter. In some embodiments, the method further comprises rotating the housing of the hub to releasably lock the axial location of the circulatory support catheter relative to the insertion catheter.

Described herein are methods and devices relating to a peel-away catheter configured to slidably receive at least a portion of a MCS system, the peel-away catheter comprising a catheter shaft configured to be splitable and/or separable into multiple elongate shaft portions. A proximal portion of the catheter shaft can be configured to mate with an insertion tool for the MSC system. In some instances, a medical delivery system can comprise an introducer sheath, an insertion tool, and a peel-away catheter comprising a catheter shaft having a portion disposed within the introducer sheath while a proximal portion of the catheter shaft is engaged with the insertion tool. The medical delivery system can comprise a spacer configured to axially space the insertion tool from the introducer sheath while the catheter shaft is engaged with the insertion tool.

Described herein are methods and devices relating to a peel-away catheters configured to slidably receive at least a portion of a MCS system, and be used independently of an insertion tool. For example, the MCS system can be slidably disposed through the peel-away catheter for navigation to a target location without using a separate insertion tool. In some instances, the peel-away catheters can comprise a catheter shaft extending distally from a catheter hub. The catheter hub may or may be not a peel-away hub. In some instances, the catheter hub is not a peel-away hub. For example, the catheter shaft can be peeled and/or removed from around the MCS system after the catheter shaft is separated, disengaged from and/or decoupled from the catheter hub.

Also described herein are medical delivery systems comprising an insertion tool comprising an insertion tool hub and an insertion tool shaft extending distally from the insertion tool hub, and a peel-away catheter comprising a catheter shaft configured to be separable into a plurality of elongate shaft portions, and the catheter shaft comprising a proximal portion configured to mate with a distal portion of the insertion tool shaft.

Also described herein are medical delivery systems, wherein the proximal portion of the catheter shaft comprises an insertion tool mating portion configured to be disposed over and have an interference fit with an exterior surface of the distal portion of the insertion tool shaft.

Also described herein are medical delivery systems, wherein the insertion tool mating portion comprises a flared configuration.

Also described herein are medical delivery systems, wherein the peel-away catheter further comprises a first and second operator engagement handle coupled to respective portions of the proximal portion of the catheter shaft for engagement by an operator to separate the catheter shaft into the plurality of elongate shaft portions.

Also described herein are medical delivery systems, wherein the peel-away catheter and the insertion tool are configured to slidably receive respective portions of a Mechanical Circulatory Support (MCS) system.

Also described herein are medical delivery systems, wherein corresponding portions of the Mechanical Circulatory Support (MCS) system are configured to be disposed through respective delivery lumens of the insertion tool and peel-away catheter, a distal tip of the Mechanical Circulatory Support (MCS) system being configured to be disposed distally of the catheter shaft.

Also described herein are medical delivery systems, further comprising a support sleeve comprising slit and configured to be disposed around a portion of a shaft of the Mechanical Circulatory Support (MCS) system, a corresponding portion of a delivery lumen of the catheter shaft being configured to slidably receive the support sleeve and the portion of the shaft of the Mechanical Circulatory Support (MCS) system.

Also described herein are medical delivery systems, further comprising an introducer sheath comprising an introducer sheath shaft extending distally from an introducer sheath hub, and wherein at least a portion of the catheter shaft is configured to be slidably disposed within the introducer sheath shaft and the introducer sheath hub while the proximal portion of the catheter shaft is mated with the distal portion of the insertion tool shaft.

Also described herein are medical delivery systems, further comprising a spacer configured to engage with a spacer engagement feature of the introducer sheath hub and a spacer engagement feature of the insertion tool hub to axially space and align the introducer sheath hub and the insertion tool hub.

Also described herein are medical delivery systems, wherein the spacer comprises a distal portion configured to engage with the spacer engagement feature of the introducer sheath hub, a proximal portion configured to engage with the spacer engagement feature of the insertion tool hub, and a medial portion extending between and perpendicular to the distal and proximal portions, the distal portion and proximal portion each comprising a concave edge configured to mate with a respective groove of the spacer engagement feature of the introducer sheath hub or insertion tool hub.

Also described herein are medical delivery systems comprising an introducer sheath including an introducer sheath shaft extending distally from an introducer sheath hub, a peel-away catheter including a catheter shaft extending distally from a catheter hub, the introducer sheath and peel-away catheter being configured to slidably receive a Mechanical Circulatory Support (MCS) system. The system can include a spacer configured to engage with the introducer sheath hub and the catheter hub to axial space the introducer sheath hub from the catheter hub.

Also described herein are medical delivery systems, wherein the peel-away catheter comprises a first elongate member comprising a first elongate portion having at least a portion configured to be slidably disposed in a first elongate member lumen extending along a first longitudinal portion of a shaft wall of the catheter shaft while the peel-away catheter is in an unpeeled configuration. The peel-away catheter can include a second elongate member comprising a second elongate portion having at least a portion configured to be slidably disposed in a second elongate member lumen extending along a second longitudinal portion of the shaft wall of the catheter shaft while the peel-away catheter is in the unpeeled configuration, the first and second elongate portions being configured to be pulled laterally to cut through corresponding shaft wall portions between the first and second elongate member lumens and an exterior surface of the shaft wall for separating the shaft wall into first and second elongate shaft wall portions.

Also described herein are medical delivery systems, wherein the first elongate portion is opposingly disposed around a circumference of the catheter shaft relative to the second elongate portion.

Also described herein are medical delivery systems, wherein the peel-away catheter comprises a first proximal shaft tab coupled to a proximal portion of the first elongate shaft wall portion, and a second proximal shaft tab coupled to a proximal portion of the second longitudinal shaft wall portion, the first and second proximal shaft tabs being configured to be pulled laterally to tear corresponding shaft wall portions between the first and second elongate member lumens and an interior surface of the shaft wall for separating the shaft wall into the first and second elongate shaft wall portions.

Also described herein are medical delivery systems, wherein the first and second proximal shaft tabs are disposed at opposing positions around a circumference of the catheter shaft.

Also described herein are medical delivery systems, wherein the peel-away catheter comprises a first elongate member comprising a first elongate portion, a second elongate member comprising a second elongate portion, and a first and second elongate wall portions. While the catheter shaft is in an unpeeled configuration, the first elongate portion can be between a first edge of the second elongate shaft wall portion and a second edge of the first elongate shaft wall portion, and the second elongate portion can be between a first edge of the first elongate shaft wall portion and a second edge of the second elongate shaft wall portion, the first and second elongate portions being configured to be pulled laterally to separate the first and second elongate portions from the first and second elongate shaft wall portions.

Also described herein are medical delivery systems, wherein the first elongate portion is opposingly disposed around a circumference of the catheter shaft relative to the second elongate portion, and the first elongate shaft wall portion is opposingly disposed around the circumference of the catheter shaft relative to the second elongate shaft wall portion.

Also described herein are medical delivery systems, wherein the peel-away catheter comprises an elongate member comprising an elongate portion, and wherein a shaft wall of the catheter shaft comprises a first interlocking portion comprising a first plurality of mating portions along a longitudinal portion of the catheter shaft, and a second interlocking portion comprising a second plurality of mating portions along the longitudinal portion of the catheter shaft and configured to mate with the first interlocking portion. The peel-away catheter can include an elongate member comprising an elongate portion, corresponding portions of the elongate portion being configured to be slidably disposed within respective elongate member lumen portions of each of the first and second plurality of mating portions of the first and second interlocking portions.

Also described herein are medical delivery systems, wherein mating edges of the first and second interlocking portions have a rectangular wave shape.

Also described herein are medical delivery systems, wherein the elongate portion is configured to be withdrawn from the elongate member lumen portions to allow the first and second interlocking portions to separate.

Also described herein are medical delivery systems, wherein the peel-away catheter comprises an elongate member configured to fasten together edges of a shaft wall of the catheter shaft to maintain the catheter shaft in an unpeeled configuration.

Also described herein are medical delivery systems, wherein the shaft wall comprises a first edge portion comprises a plurality of first openings at respective locations along a longitudinal portion of the first edge portion, and a second edge portion comprising a plurality of second openings at respective locations along a longitudinal portion of the second edge portion. The elongate member can include an elongate portion, corresponding portions of the elongate portion being configured to be disposed through the plurality of first and second openings in alternating fashion to fasten together the first edge portion and the second edge portion together.

Also described herein are medical delivery systems, wherein the elongate portion is configured to be withdrawn from the plurality of first and second openings to allow the first and second edge portions to separate.

Also described herein are medical delivery systems, wherein the catheter shaft is configured to be in a rolled configuration while the catheter shaft is in the unpeeled configuration.

Also described herein are medical delivery systems, wherein a shaft wall of the catheter shaft comprises a first overlapping wall portion, and a second overlapping wall portion, and while the catheter shaft is in the rolled configuration, the first overlapping wall portion being configured to be over and in contact with the second overlapping wall portion along a longitudinal portion of the shaft wall.

Also described herein are medical delivery systems, wherein the peel-away catheter comprises a proximal shaft tab coupled to a proximal portion of a shaft wall of the catheter shaft, the proximal shaft tab is coupled at a position circumferentially spaced from the first and second overlapping wall portions.

Also described herein are medical delivery systems, wherein the proximal shaft tab is opposingly oriented around a circumference of the catheter shaft relative to the first and second overlapping wall portions.

Also described herein are medical delivery systems, wherein at least one of an outer diameter of the catheter shaft and a diameter of the shaft delivery lumen of the catheter shaft is configured to conform to that of corresponding portions of the MCS system disposed therethrough.

Also described herein are medical delivery systems, wherein the spacer is configured to engage with a spacer engagement feature of the introducer sheath hub and a spacer engagement feature of the catheter hub to axially space and align the introducer sheath hub and the catheter hub.

Also described herein are medical delivery systems, wherein the spacer comprises a distal portion configured to engage with the spacer engagement feature of the introducer sheath hub, a proximal portion configured to engage with the spacer engagement feature of the catheter hub, and a medial portion extending between and perpendicular to the distal and proximal portions, the distal portion and proximal portion each comprising a concave edge configured to mate with a respective groove of the spacer engagement feature of the introducer sheath hub or catheter hub.

Also described herein are medical delivery systems, wherein the catheter hub is not a peel-away hub.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure.

FIG. 1 is a cross sectional rendering of an embodiment of a mechanical circulatory support (MCS) device of the present disclosure carried by a catheter and positioned across an aortic valve via a femoral artery access.

FIG. 2 schematically illustrates an MCS system inserted into the body via the access pathway from the femoral artery to the left ventricle according to some embodiments.

FIG. 3 is a side elevational view of an embodiment of an MCS system that may incorporate the various features described herein.

FIG. 4 is the system of FIG. 3, with the introducer sheath removed and including an insertion tool and a guidewire loading aid.

FIG. 5 shows an introducer kit having an introducer sheath and dilator, that may be used with the various MCS systems and methods described herein.

FIG. 6 shows an embodiment of a placement guidewire that may be used with the various MCS systems and methods described herein.

FIG. 7 is a partial perspective view of a distal, pump region of the MCS device.

FIGS. 8A and 8B are a side elevational view and close up detail view respectively of a distal region of the MCS device, showing the guidewire guide tube defining the guidewire path and the guidewire back loading aid in place.

FIGS. 9A and 9B are respectively a side view of a pump region of the MCS device and a cross sectional view through the impeller region of the MCS device.

FIG. 10 is a partial cross sectional view, through an impeller and magnetic coupling region, of an embodiment of a pump that may be used with the various MCS systems described herein.

FIGS. 11A, 11B, and 11C are a front elevational view, a rear perspective view, and a side perspective view, respectively, of an MCS controller.

FIG. 12 is a side elevational view of an introducer sheath and hub that may be used with the various MCS systems described herein.

FIGS. 13A-13E are various views of an embodiment of an insertion tool that may be used with the various MCS systems described herein.

FIGS. 14A-14E are various views of an embodiment of an insertion catheter that may be used with the various MCS systems described herein.

FIGS. 15A-15C are various views of a tubular body of an insertion catheter according to FIGS. 14A-14E.

FIGS. 16A-16D are various views of an MCS device carried by a catheter separate of and placed within the insertion catheter according to FIGS. 14A-14E.

FIGS. 17A-17C are various views of an MCS device carried by a catheter placed within the insertion catheter according to FIGS. 14A-14E and partially within an introducer sheath.

FIG. 18 illustrates an MCS device carried by a catheter placed within the insertion catheter according to FIGS. 14A-14E not inserted, partially inserted, and fully inserted within an introducer sheath.

FIG. 19A is a side view of a delivery assembly comprising a peel-away catheter and an insertion tool configured to facilitate delivery of an MCS system to a target position.

FIG. 19B is a side view of the peel-away catheter described with reference to FIG. 19A.

FIG. 20 is a side view of a medical delivery system comprising the peel-away catheter described with reference to FIGS. 19A and 19B, an insertion tool, an introducer sheath, and a spacer configured to axially space the insertion tool from the introducer sheath.

FIG. 21A is an exploded perspective view of an example of a peel-away catheter in an unpeeled configuration, and comprising elongate members configured to cut through shaft wall portions of a catheter shaft. FIG. 21B is a perspective view of the catheter shaft described with reference to FIG. 21A being peeled and/or removed from around the Mechanical Circulatory Support (MCS) system disposed through the peel-away catheter.

FIG. 22A is an exploded perspective view of an example of a peel-away catheter in an unpeeled configuration, and comprising elongate members configured to allow separation of a catheter shaft into a plurality of elongate shaft portions. FIG. 22B is a more detailed view of a proximal portion of the catheter shaft described with reference to FIG. 22A. FIG. 22C is a perspective view of the catheter shaft described with reference to FIGS. 22A and 22B being peeled and/or removed from around the Mechanical Circulatory Support (MCS) system disposed through the peel-away catheter.

FIG. 23A is an exploded perspective view of an example of a peel-away catheter comprising interlocking portions configured to be mated together while in an unpeeled configuration. FIG. 23B is a perspective view of the catheter shaft described with reference to FIG. 23A being peeled and/or removed from around the Mechanical Circulatory Support (MCS) system disposed through the peel-away catheter.

FIG. 24A is an exploded perspective view of an example of a peel-away catheter comprising an elongate member configured to fasten together edges of a shaft wall of a catheter shaft. FIG. 24B is a perspective view of the catheter shaft described with reference to FIG. 24A being peeled and/or removed from around the Mechanical Circulatory Support (MCS) system disposed through peel-away catheter.

FIG. 25A is an exploded perspective view of an example of a peel-away catheter assuming a rolled configuration while in an unpeeled configuration. FIG. 25B is a perspective view of the catheter shaft described with reference to FIG. 25A being peeled and/or removed from around the Mechanical Circulatory Support (MCS) system disposed through the peel-away catheter.

FIG. 26 is side cross-sectional view of an example of peel-away catheter comprising a catheter shaft with an outer diameter that varies along a longitudinal dimension of the catheter shaft.

FIG. 27 is side cross-sectional view of an example of peel-away catheter comprising a catheter shaft with an outer diameter that is similar or the same along a longitudinal dimension of the catheter shaft.

FIG. 28 is a side view of a medical delivery system comprising a peel-away catheter, an introducer sheath, and a spacer configured to axially space the peel-away catheter from the introducer sheath.

FIGS. 29A and 29B illustrate perspective and cross-section views of the MCS device placed within an enlarged portion of a relatively longer insertion catheter and fully inserted within a relatively shorter introducer sheath, with the enlarged portion containing the MCS device located beyond the bifurcation and protruding slightly distally out of the introducer sheath prior to deployment of the MCS device.

While the above-identified drawings set forth presently disclosed embodiments, other embodiments are also contemplated, as noted in the detailed description. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following detailed description is directed to certain specific embodiments of the development. In this description, reference is made to the drawings wherein like parts or steps may be designated with like numerals throughout for clarity. Reference in this specification to “one embodiment,” “an embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments. Reference will now be made in detail to embodiments of the invention, examples of which are illustrated in the accompanying drawings. Wherever possible, the same reference numbers will be used throughout the drawings to refer to the same or like parts.

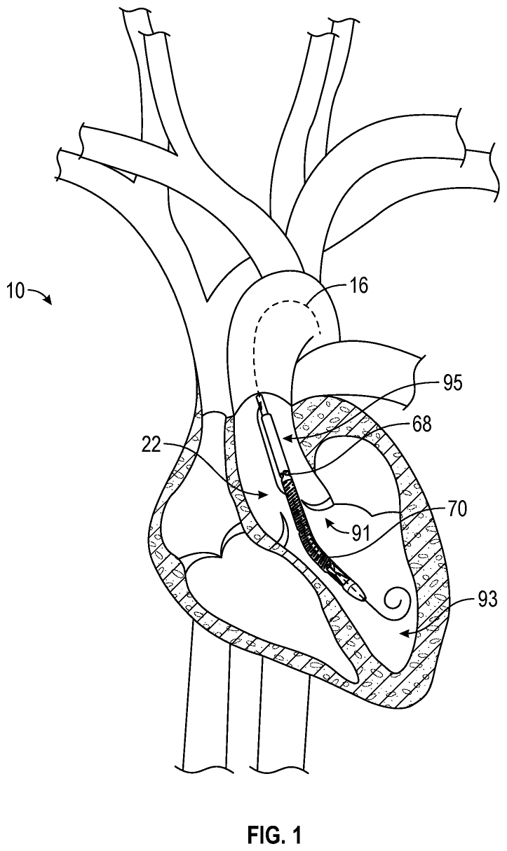

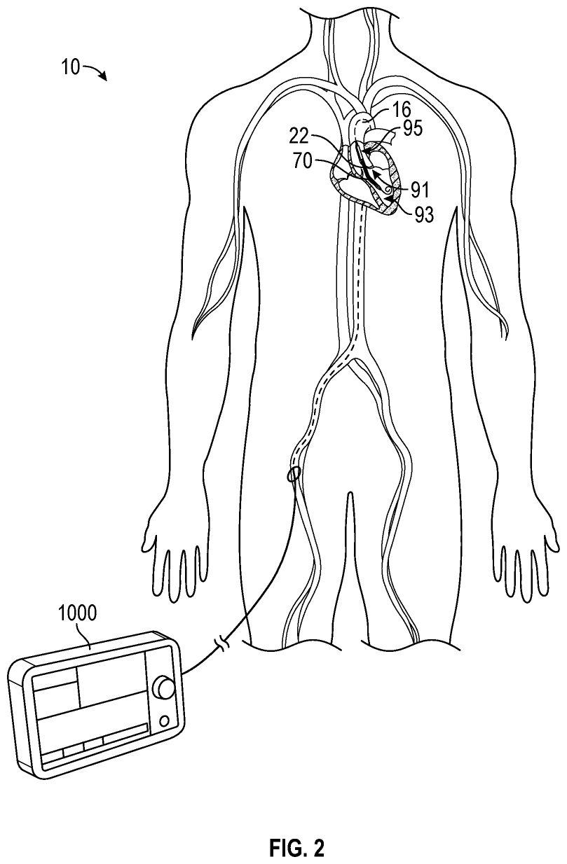

FIG. 1 is a schematic of a distal end of an embodiment of a mechanical circulatory support (MCS) system 10 having a pump 22 mounted on the tip of a catheter 16 placed in the heart. FIG. 2 schematically illustrates an MCS system inserted into the body via an access pathway from a femoral artery to the aorta to the left ventricle according to some embodiments. Some features of the MCS system 10 will be described with respect to FIGS. 1 and 2, with further detail of various features provided elsewhere herein.

Various embodiments of the MCS system 10 are described herein having various features. In some embodiments, the MCS system 10 may include a temporary (e.g., generally no more than about 6 hours, or in some embodiments no more than about 3 hours, no more than about 4 hours, no more than about 7 hours, no more than about 8 hours, no more than about 9 hours, or no more than about 10 hours) left ventricular support device or pump, also referred to as an MCS pump, MCS device, or circulatory support device. The device may be used during high-risk percutaneous coronary intervention (PCI) performed in elective or urgent, hemodynamically stable patients with severe coronary artery disease and/or depressed left ventricular ejection fraction, e.g. when a heart team, including a cardiac surgeon, has determined high risk PCI is the appropriate therapeutic option. The pump can be placed across the aortic valve via a single femoral arterial access.

In some embodiments, the MCS system 10 may include a longer-term pump 22, for example as therapy for cardiogenic shock. The MCS system 10 may include the pump 22 having a first magnet rotated by a motor within a sealed motor housing. An impeller with a second magnet may partially surround the first magnet external to the motor housing. Rotation of the first magnet causes, via magnetic communication, the second magnet and impeller to rotate.

In some embodiments, the MCS system 10 may include an insertion tool having a tubular body and configured to axially movably receive the circulatory support device (which can also be referred to herein as an MCS pump or an MCS device) and its catheter 16 (which can also be referred to herein as a catheter shaft, a pump shaft, or an MCS shaft). An introducer sheath (which can also be referred to herein as an access sheath and/or an expandable sheath), having a tubular body, may be configured to axially movably receive the insertion tool. The insertion tool may protect the circulatory support device, for example during insertion in and through the introducer sheath and any of its hemostatic valve(s).

In some embodiments, the MCS system 10 may include a low-profile axial rotary blood pump mounted on the catheter 16, such as an 8 French (Fr) catheter. When in place, the MCS pump 22 may be driven by an MCS controller 1000 to provide up to about 4.0 liters/minute of partial left ventricular support, which may be at about 60 mm Hg. No system purging is needed due to improved bearing design and sealed motor. The MCS system 10 or portions thereof may be visualized fluoroscopically, eliminating the need for placement using sensors.

In some embodiments, the MCS system 10 may include an introducer sheath. The sheath may be expandable. The expandable sheath may allow for example an 8 to 10 Fr initial access size for easy insertion and closing, expandable to allow introduction of 14 Fr, 16 Fr, and 18 Fr pump devices, and return to a narrower diameter around the 8 Fr catheter 16 and insertion tool once the pump 22 has passed. This feature may allow passage of the pump 22 through vasculature while minimizing shear force within the blood vessel, advantageously reducing risk of bleeding and healing complications. Distention or stretching of an arteriotomy may be done with radial stretching with minimal shear, which is less harmful to the vessel. Access may be accomplished via transfemoral, transaxillary, transaortal, or transapical approach. In some embodiments, an expandable sheath may allow 8 to 16 Fr (e.g., 8 to 10.5) Fr initial access size for easy insertion and closing, expandable to allow introduction of at least about a 14 Fr, a 16 Fr, an 18 or 19 Fr device.

In some embodiments, an inlet tube 70 of the pump 22 extends across the aortic valve 91. An impeller may be located at the outflow section 68 (also referred to as a pump outlet herein) of the inlet tube 70, drawing blood from the left ventricle 93 through the inlet tube 70 and ejecting it out the outflow section 68 into the ascending aorta 95. The motor may be mounted directly proximal to the impeller in a sealed housing, eliminating the need to purge or flush the motor prior to or during use. This configuration provides hemodynamic support during high-risk PCI, with sufficient time and safety for a complete revascularization via a minimally invasive approach (rather than an open surgical procedure).

In some embodiments, the MCS system 10 actively unloads the left ventricle by pumping blood from the ventricle into the ascending aorta and systemic circulation. When in place, the MCS device 22 may be driven by the complementary MCS Controller 1000 to provide between 0.4 l/min up to 4.0 l/min of partial left ventricular support. The MCS system 10 may eliminate the need for motor flushing, provide increased flow performance up to 4.0 l/min at 60 mmHg with acceptably safe hemolysis due to a computational fluid dynamics (CFD) optimized impeller that minimizes shear stress. When in place, the MCS device 22 can be driven by the complementary MCS controller 1000 to provide between 0.4 l/min up to 6.0 l/min of partial left ventricular support. In some embodiments, the MCS device 22 can be driven by the complementary MCS controller 1000 to provide between 0.6 l/min up to 6.0 l/min of partial left ventricular support. A range between 0.6 l/min up to 6.0 l/min may allow for 10 equidistant flow levels, for example.

In some embodiments, the MCS system 10 may include a MCS device 22 comprising an 18 to 19 Fr axial rotary blood pump and inlet tube assembly mounted on the catheter 16, such as a catheter no larger than 10.5 Fr. When in place, the MCS pump 22 can be driven by a complementary MCS controller 1000, which may provide at least about 4.0 or 5.0 and up to about 6.0 liters/minute of partial left ventricular support, at about 60 mm Hg pressure differential. In some embodiments of the pump 22, no system purging is needed due to the encapsulated motor and magnetic bearing design.

In general, the overall MCS system 10 may include a series of related subsystems and accessories, including one or more of the following: The MCS system 10 may include a pump, shaft, proximal hub, insertion tool (or insertion catheter as described later herein), introduction sheath, proximal cable, infection shield, guidewire guide tube and/or guidewire aid. The pump 22 may be provided sterile. An MCS shaft may contain the electrical cables and a guidewire lumen for over-the-wire insertion. The proximal hub can contain a guidewire outlet with a valve to maintain hemostasis and connect the MCS shaft to the proximal cable, that connects the pump 22 to the controller 1000. The proximal cable 28 may be 3.5 m (approx. 177 inch) in length and extend from a sterile field 5 to a non-sterile field 3 where the controller 1000 is located. An insertion tool may be provided pre-mounted on or otherwise pre-coupled to the MCS device and its catheter shaft to facilitate the insertion of the pump and catheter shaft into an introducer sheath and to protect the inlet tube and the valves from potential damage or interference when passing through the introducer sheath. A peel-away guidewire aid may be pre-mounted on the MCS Device to facilitate the insertion of a guidewire, such as an 0.018″ placement guidewire, into the pump 22 and into the MCS catheter shaft 16, optionally with the insertion tool also pre-mounted such that the guidewire guide tube may pass at least in part through a space between the MCS device and the insertion tool. A 3 m, 0.018″ placement guidewire may be used, having a soft coiled pre-shaped tip for atraumatic wire placement into the left ventricle. The guidewire may be provided sterile. A 14 Fr or 16 Fr introducer sheath may be used with a usable length of 275 mm to maintain access into the femoral artery and provide hemostasis for a 0.035″ guidewire, a diagnostic catheter, the 0.018″ placement guidewire, and the insertion tool. The housing of the introducer sheath may be designed to accommodate the insertion tool with the MCS device and its shaft mounted therein. The introducer sheath can be provided sterile. An introducer dilator may be compatible with the introducer sheath to facilitate atraumatic insertion of the introducer sheath into the femoral artery. The introducer dilator can be provided sterile. The controller 1000 may be used which drives and operates the pump 22, observes its performance and condition, and/or provides error and status information. The powered controller 1000 may be designed to support at least about 12 hours of continuous operation and can contain a basic interface to indicate and adjust the level of support provided to the patient. Moreover, the controller 1000 may provide an optical and audible alarm notification in case the system detects an error during operation. The controller 1000 may be provided non-sterile and be contained in an enclosure designed for cleaning and re-use outside of the sterile field 5. The controller 1000 enclosure may contain a socket into which the extension cable is plugged.

In some embodiments, the pump 22 (which may also be referred to as a ventricular support device (VSD) or mechanical circulatory support device) of the present disclosure may be a temporary (generally no more than about 6 days) left ventricle support device for enhancing cardiac output in cardiogenic shock patients such as caused by acute ST elevation myocardial infarction. The pump 22 may be placed across the aortic valve, typically via transvascular access, to pump blood from the left ventricle to the ascending aorta.

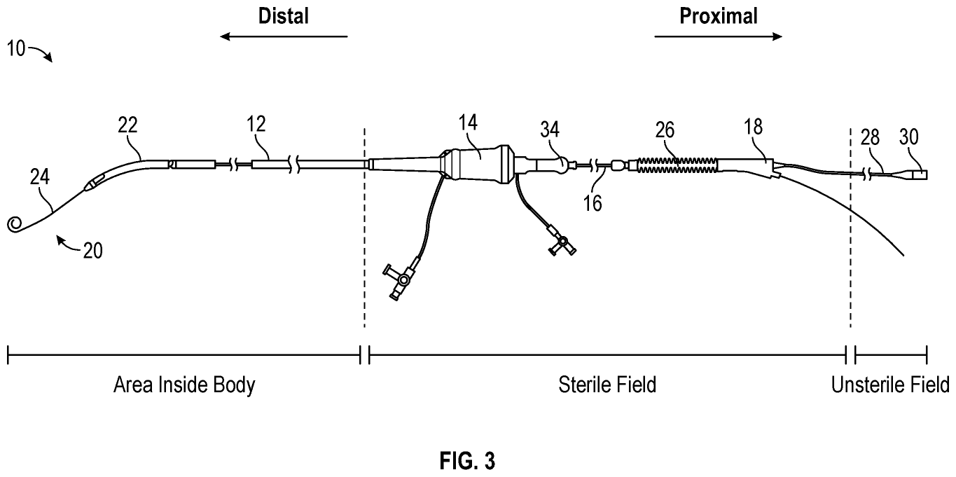

Referring to FIG. 3, there is illustrated an overall MCS system 10 in accordance with some embodiments, subcomponents of which will be described in greater detail below. For reference, the “distal” and “proximal” directions are indicated by arrows in FIGS. 3, 4 and 8A. “Distal” and “proximal” as used herein have their usual and customary meaning, and include, without limitation, a direction more distant from an entry point of the patient's body as measured along the delivery path, and away a direction less distant from an entry point of the patient's body as measured along the delivery path, respectively.

The system 10 may include an introducer sheath 12 having a proximal introducer hub 14 with a central lumen for axially movably receiving an MCS shaft 16 (the MCS shaft may also be referred to as a catheter, catheter shaft, or a shaft herein). The MCS shaft 16 may extend between a proximal hub 18 and a distal end 20 of the system 10, with a guidewire 24 extending therefrom. The proximal hub 18 may be provided with an integrated Microcontroller or memory storage device for device identification and tracking of the running time, which could be used to prevent overuse to avoid excessive wear or other technical malfunction. The microcontroller or memory device could disable the device, for example to prevent using a used device. They could communicate with the controller 1000, which could display information about the device or messages about its usage. An atraumatic cannula tip with radiopaque material allows the implantation/explantation to be visible under fluoroscopy.

The pump 22 can comprise a tubular housing. The tubular housing of the pump 22 is used broadly herein and may include any component of the pump 22 or component in the pump region of the system, such as an inlet tube, a distal endpiece, a motor housing, other connecting tubular structures, and/or a proximal back end of the motor housing. The pump 22, for example the tubular housing, is carried by a distal region of the MCS shaft 16. The system 10 is provided with at least one central lumen for axially movably receiving the guidewire 24. The proximal hub 18 can additionally be provided with an infection shield 26. A proximal cable 28 can extend between the proximal hub 18 and a connector 30 for releasable connection to a controller 1000 typically outside of the sterile field 3, to drive the pump 22.

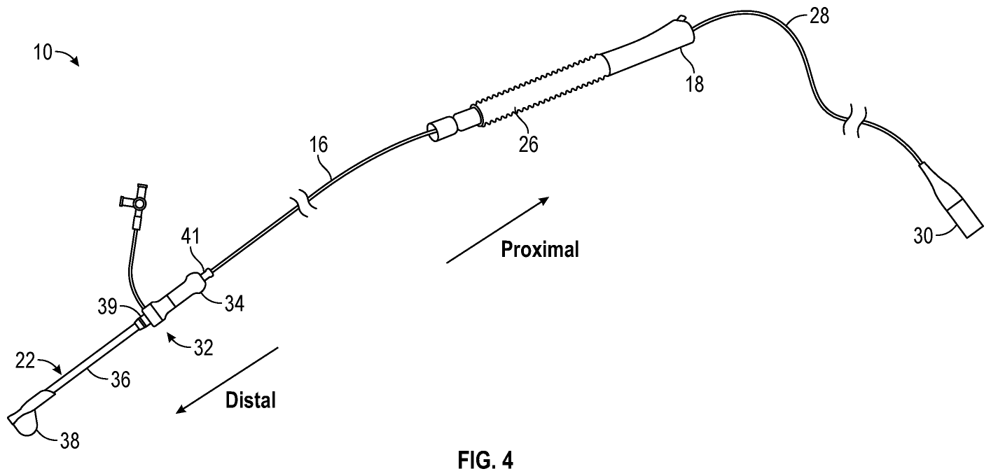

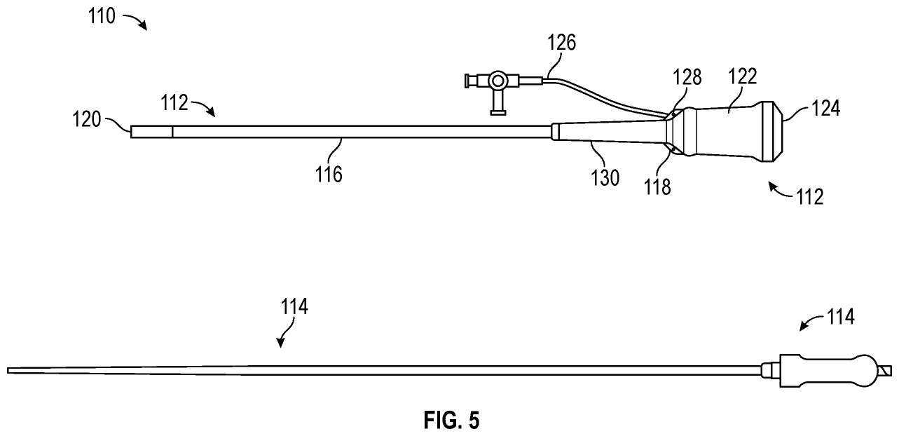

Referring to FIG. 4, the system 10 may additionally include an insertion tool 32, having an elongate tubular body 36 having a length within the range of from about 85 mm to about 160 mm (e.g., about 114 mm) which may be adapted to span the length of the hub 122 and bend relief 130 of the introducer sheath 112 (see FIG. 5) and an inside diameter within the range of from about 4.5 mm to about 8.0 mm (e.g., about 5.55 mm), extending distally from a proximal hub 34. The tubular body 36 includes a central lumen adapted to, in some embodiments, axially movably receive the MCS shaft 16 and pump 22 there through, and sufficient collapse resistance to maintain patency when passed through the hemostatic valves of the introducer sheath. As illustrated in FIG. 4, the tubular body 36 can be configured as a generally linearly straight tube. Also shown, the distal end of the tubular body 36 can taper in the distal direction such that its inner and outer diameter narrows in the distal direction. The pump 22 can be positioned within the tubular body 36, such as to facilitate passage of the pump 22 through the hemostatic valve(s) on the proximal end of an introducer hub 14 and/or the entire length of the introducer sheath 112. A marker 37 (FIG. 7) can be provided on the MCS shaft 16 spaced proximally from the distal tip 64 such that as long as the marker 37 is visible on the proximal side of the hub 34, the clinician knows that the pump is within the tubular body 36.

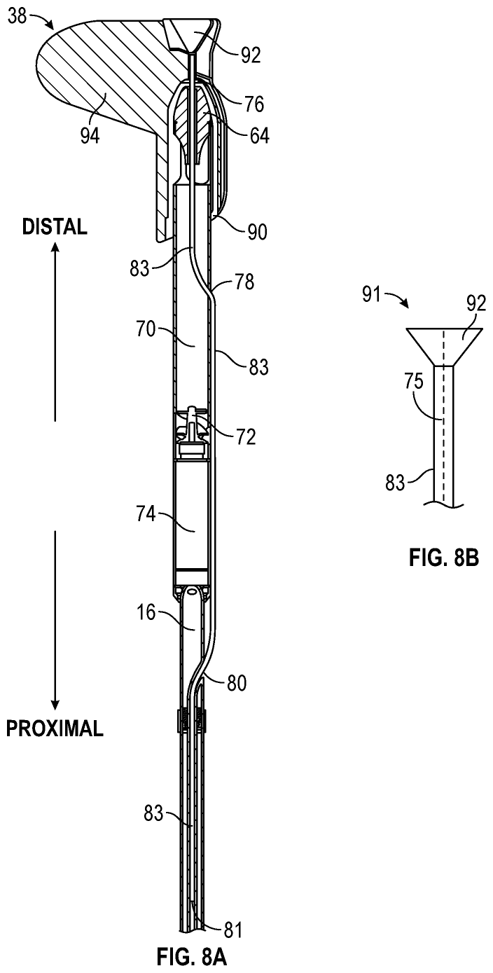

The hub 34 of the insertion tool 32 may be provided with a first engagement structure 39 for engaging a complimentary second engagement structure on the introducer sheath (e.g., insertion sheath 14 and/or 112) to lock the insertion tool into the introducer sheath. For example, the first engagement structure 39 can be disposed at the distal end of the hub 34, such that when the insertion tool 32 is inserted into the introducer sheath it engages with the complementary second engagement structure on the proximal end of the introducer sheath when the insertion tool is fully inserted/docked into the introducer sheath. As shown, such a first engagement structure can have an oblong or oval shape cross-sectional shape, although other configurations are possible, preferably ones that can prevent relative rotational movement between the insertion tool and the introducer sheath when the insertion tool is docked (e.g., fully inserted) with the introducer sheath. The hub 34 may be connected with the infection shield 26 via a connection 41, such as a knob or button that connects via force-fit, screw, or other means. The hub 34 may also be provided with a locking mechanism for clamping onto the shaft 16 to prevent the shaft 16 from sliding proximally or distally through the insertion tool once the MCS device has been positioned at the desired location in the heart (e.g., to prevent axial movement of the catheter shaft 16 and MCS device 22). Additionally or alternatively, the locking mechanism of the insertion tool 32 can be configured to prevent the shaft 16 from rotating relative to the insertion tool. The locking mechanism may be actuated by twisting one or more parts (for example, two parts) of the hub 34. Other actuation means may also be possible. The hub 34 may additionally be provided with a hemostasis valve to seal around the shaft 16. In some embodiments, the hub 34 may accommodate passage of the larger diameter MCS device which includes the pump. In one commercial presentation of the system, the MCS device as packaged is pre-positioned within the insertion tool and the guidewire aid is pre-loaded within the MCS device and shaft 16, as illustrated in FIG. 4. In some examples, the MCS device is configured to be prepositioned in the tube 36 and advanced distally. In such a configuration, the lumen in the hub 34 may be smaller than the MCS device and only the shaft 16 may be configured to pass through the hub 34. When removing the pump from the body, the MCS device may be pulled into the tube 36 and then the insertion tool may be pulled out of the introducer sheath with the pump in the tube 36. Further details of a guidewire aid 38 are discussed, for example, with reference to FIGS. 8A and 8B.

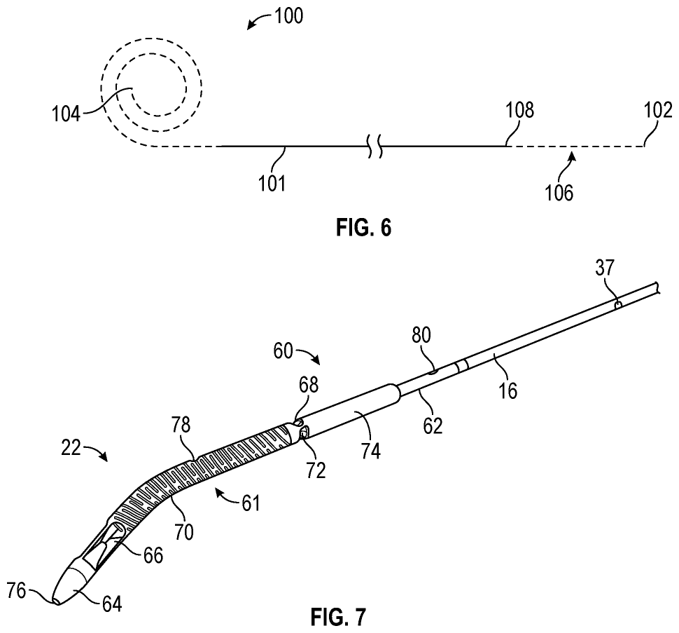

Referring to FIGS. 5 and 6, an introducer kit 110 may include a guidewire 100, an introducer sheath 112, a dilator 114, and/or a guidewire aid 38, such as discussed with reference to FIGS. 8A and 8B. The guidewire 100 and introducer sheath 112 may correspond to guidewire 24 and introducer sheath 12 discussed above. The guidewire 100 (e.g., 0.018″ placement guidewire) may comprise an elongate flexible body 101 extending between a proximal end 102 and a distal end 104. A distal zone of the body 101 may be pre-shaped into a J tip or a pigtail, as illustrated in FIG. 6, to provide an atraumatic distal tip. A proximal zone 106 may be configured to facilitate threading through the MCS device and may extend between the proximal end 102 and a transition 108. The proximal zone 106 may have an axial length within the range of from about 100 mm to about 500 mm (e.g., about 300 mm).

The introducer kit 110 may comprise the introducer sheath 112 and/or the dilator 114. The introducer sheath 112 may comprise an elongate tubular body 116, extending between a proximal end 118 and a distal end 120. The tubular body 116 can terminate proximally in a proximal hub 122. Optionally, the tubular body 116 can be expandable (e.g., to accommodate passage of the MCS device 22 therethrough) or can be peeled apart. The proximal hub 122 includes a proximal end port 124 in communication with a central lumen extending throughout the length of the tubular body 116 and out through a distal opening, configured for axially removably receiving the elongate dilator 114. Proximal hub 122 may additionally be provided with a side port 126, at least one and optionally two or more attachment features such as an eye 128 to facilitate suturing to the patient, and at least one and optionally a plurality of hemostasis valves for providing a seal around a variety of introduced components such as a standard 0.035″ guidewire, a 5 Fr or 6 Fr diagnostic catheter, an 0.018″ placement guidewire 100, the shaft 16, and the insertion tool 32. Proximal hub 122 may have a lock for preventing axial movement of the insertion tool 32 and/or the dilator 114. Furthermore, the proximal hub 122 (e.g., the proximal end of the hub) can include the secondary engagement structure for engaging with the first engagement structure 39 of the insertion tool 32.

FIG. 7 illustrates additional details of a distal pump region 60 of the MCS system showing the device or pump 22 and a distal portion of the catheter shaft 16. The pump zone or region 60 extends between a bend relief 62 at the distal end of shaft 16 and a distal tip 64. The pump 22 can include a tubular housing 61, which may include an inlet tube 70, a distal tip 64, and/or a motor housing 74. The tubular housing 61 may include one or more pump inlets 66 and/or outlets 68, which may be part of the inlet tube 70, or part of other structures such as an intermediate structure joining a proximal end of the inlet tube 70 to the motor housing 74. A guidewire guide aid, as further described herein, may extend into and out of various components of the system, such as the tubular housing 61 of the pump 22 and/or the catheter shaft 16 (e.g., bend relief 62).

The pump inlet 66 comprises one or more windows or openings in fluid communication with a pump outlet 68 (also referred to as an outflow section herein) by way of a flow path extending axially through the inlet tube 70. The pump inlet may be positioned at about the transition between the inlet tube and the proximal end of distal tip 64, and in any event is generally within about 5 cm or 3 cm or less from the distal port 76.

In some embodiments, the distal tip 64 is radiopaque. For example, the distal tip may be made from a polymer containing a radiopacifier such as barium sulfate, bismuth, tungsten, iodine. In some embodiments, an entirety of the MCS device is radiopaque. In some embodiments, a radiopaque marker is positioned on the inlet tube 70 between the pump outlet 68 and the guidewire port 78 to indicate the current position of the MCS device relative to the aortic valve 91.

The inlet tube 70 may comprise a highly flexible slotted (e.g., laser cut) metal (e.g., Nitinol) tube having a polymeric (e.g., Polyurethane) tubular layer to isolate the flow path. Inlet tube 70 may have an axial length within the range of from about 60 mm to about 100 mm and in one implementation may be about 67.5 mm. The outside diameter of the inlet tube 70 may typically be within the range of from about 4 mm to about 5.4 mm, and in one implementation may be about 4.66 mm. The wall thickness of the inlet tube 70 may be within the range of from about 0.05 mm to about 0.15 mm. The connections between the inlet tube 70 and the distal tip 76 and to the motor may be secured such as through the use of laser welding, adhesives, threaded or other interference fit engagement structures, or may be via press fit.

The impeller 72 may be positioned in the flow path between the pump inlet 66 and pump outlet 68. In the illustrated embodiment, the impeller 72 is positioned adjacent to the pump outlet 68. As is discussed further below, the impeller 72 may be rotationally driven by a motor contained within motor housing 74, on the proximal side of the impeller 72.

FIGS. 8A and 8B are a side cross-sectional view and a detail view respectively of the pump region showing an embodiment of a guidewire aid 38. The MCS device can be provided in either a rapid exchange or over the wire configuration. A first guidewire port 76 such as a distal-facing opening on distal face of the distal tip 64 may be in communication, via a first guidewire lumen through the distal tip 64 and at least a portion of the flow path in the inlet tube 70, with a second guidewire port 78 such as an opening extending through a side wall of the inlet tube 70, and distal to the impeller 72. This could be used for rapid exchange, with the guidewire 100 extending proximally alongside the catheter from the second guidewire port 78.

The catheter may be provided in an over the wire configuration, in which the guidewire extends proximally throughout the length of the catheter shaft 16 through a guidewire lumen therein. In the over the wire embodiment of FIGS. 7, 8A and 8B, however, the guidewire 100 exits the inlet tube 70 via second guidewire port 78, extends proximally across the outside of the impeller and motor housing, and reenters the catheter shaft 16 via a third guidewire port 80, which may be an opening in the sidewall of the catheter shaft 16 or of a proximal component of the pump, motor housing, or backend. The third guide wire port 80 may be located proximal to the motor, and, in the illustrated embodiment, is located on the bend relief 62. Third guide wire port 80 is in communication with a guide wire lumen which extends proximally throughout the length of the shaft 16 and exits at a proximal guidewire port carried by or located within the proximal hub 18 (see FIG. 4).

As shown in FIG. 8A, the pump may be provided assembled with the removable guidewire aid 38. The guidewire aid 38 may have a guidewire guide tube 83. The guide tube 83 may be a cylindrical or other closed cross-sectional shape extending axially. The guide tube 83 may be a flexible, transparent material such as polyimide. The guide tube 83 may be adapted to be peeled apart longitudinally, such as having a longitudinal slit or tear line. The inside surface of the guide tube 83 may be provided with a lubricious coating, such as PTFE. The guide tube 83 may track the intended path of the guidewire 100 from the first guidewire port 76, proximally through the tip 64 and back outside of the inlet tube via second guidewire port 78, and back into the catheter shaft 16 via the third guidewire port 80. In the illustrated implementation, the guidewire guide tube 83 extends proximally within the catheter shaft 16 to a proximal end 81 of the guide tube 83, in communication with or within the guidewire lumen which extends to the proximal hub 18. The proximal end 81 of the guide tube 83 may be positioned within about 5 mm or 10 mm of the distal end of the shaft 16, or may extend into the catheter shaft guidewire lumen for at least about 10 mm or 20 mm, such as within the range of from about 10 mm to about 50 mm. In some embodiments, the third port 80 may be located within a proximal end of the tubular housing, such as the motor housing or backend, or in any other components of the device at a location that is proximal to the impeller.

The guidewire aid 38 may have a funnel 92. The funnel 92 may be located at a distal end of the guide tube 83 and be provided pre-positioned at a distal end of the inlet tube, for example at the distal tip 64. The funnel 92 may increase in width in the distal direction, from a narrow proximal end in communication with the guide tube 83, to a wider distal opening at a distal end of the funnel 92. The funnel 92 may be conical, frustoconical, pyramidal, segmented, or other shapes. A proximal end of the funnel 92 may be attached to a distal end of the guidewire guide tube 83. The proximal end 102 of the guidewire 100 (see FIG. 6) may be inserted into the funnel 92, passing through the first (distal) guidewire port 76 and guided along the intended path by tracking inside of the guidewire guide tube 83. The guidewire guide tube 83 may then be removed by sliding the guide tube 83 distally out of the distal tip 64 and peeling it apart longitudinally, leaving the guidewire 100 in place.

The guidewire aid 38 may have a pull tab 94. In some embodiments, a distal end of the guidewire guide tube 83 is attached to the pull tab 94 of the guidewire aid 38. The pull tab 94 may be a structure capable of being gripped by a human hand, for example with a lateral, planar extension as shown. The guidewire aid 38, for example, the pull tab 94, the guide tube 83 and/or the funnel 92, may be provided with a tearable line 75, as more clearly seen in FIG. 8B. The tearable line 75 line may be an axially extending split line. The tearable line 75 may include a weakening, a slot, or a perforated linear region. Removal of the guidewire aid 38 may be accomplished such as by grasping the pull tab 94 and pulling out the guidewire tube 83 and/or funnel 92 and removing them from the guidewire 100 as they split or peel away along the split line 75, such as shown in the detailed inset 91 of FIG. 8B.

The guidewire aid 38 may include a proximal opening 90 configured to slip over and removably receive the distal tip 64 and/or struts at the distal end of the inlet tube 70 that define windows of the pump inlet 66. The guidewire guide tube 83 having a lumen therethrough is positioned within the proximal opening 90 and aligned to pass through the guidewire port 76 of the distal tip 64. The proximal opening 90 may further be configured to slip over and removably receive a distal end of tubular body 36 of an insertion tool 32 as shown in FIG. 4. The MCS system may be dimensioned so that an annular space defined between the outer surface of the MCS device—such as the inlet tube 70, motor housing 74, or MCS catheter bend relief 16—and the inner surface of the tubular body 36 of the insertion tool 32, may removably receive the guidewire guide tube 83 therein, when the MCS device, guidewire aid 38 and insertion tool 32 are assembled together.

In some embodiments, the lumen of the guidewire guide tube 83 is in communication with the distal flared opening of the funnel 92 which gets larger in cross-section in the distal direction. The guidewire aid 38 may be provided assembled on the MCS pump with the guidewire guide tube 83 pre-loaded along a guidewire path, for example into the MCS pump through port 76, through a portion of the fluid path within the inlet tube 70, out of the MCS pump through port 78, along the exterior of the MCS pump and back into the shaft 16 through port 80. This helps a user guide the proximal end of a guidewire into the funnel 92 through the guidewire path and into the guidewire lumen of the MCS shaft 16. The pull tab 94 may be provided on the guidewire aid 38 to facilitate grasping and removing the guidewire aid, including the guidewire guide tube 83, following loading of the guidewire. The guidewire aid 38 may have a longitudinal slit or tear line 75, for example along the funnel 92, proximal opening 90 and guidewire guide tube 83, to facilitate removal of the guidewire aid 38 from the MCS pump 22 and guidewire 100. Furthermore, the guidewire aid 38 and guidewire guide tube 83 can be removed from the MCS device when loaded within the tubular body 36 of the insertion tool 32, such as after a guidewire has been inserted.

The guidewire aid 38 features described herein may be used with a variety of different MCS systems and/or pump devices. The guidewire aid 38 may be used for guidewire paths that enter and exit a pump housing, as described, or that do not exit a housing. The guidewire aid 38 is described herein as being used with an MCS system configured for temporary operation for high-risk PCI procedures. The system may include a rotating impeller with a radial shaft seal and a motor rotating the impeller via a shaft extending through the seal. The guidewire aid 38 may be used with a variety of different devices. The guidewire aid 38 may also be used with a pump having a magnetic drive, where the motor rotates a first magnet within a sealed motor housing that magnetically communicates with a second magnet of the impeller that is external to the sealed housing to rotate the impeller. Thus, the guidewire aid 38 is not limited to use with only the particular pump embodiments described herein.

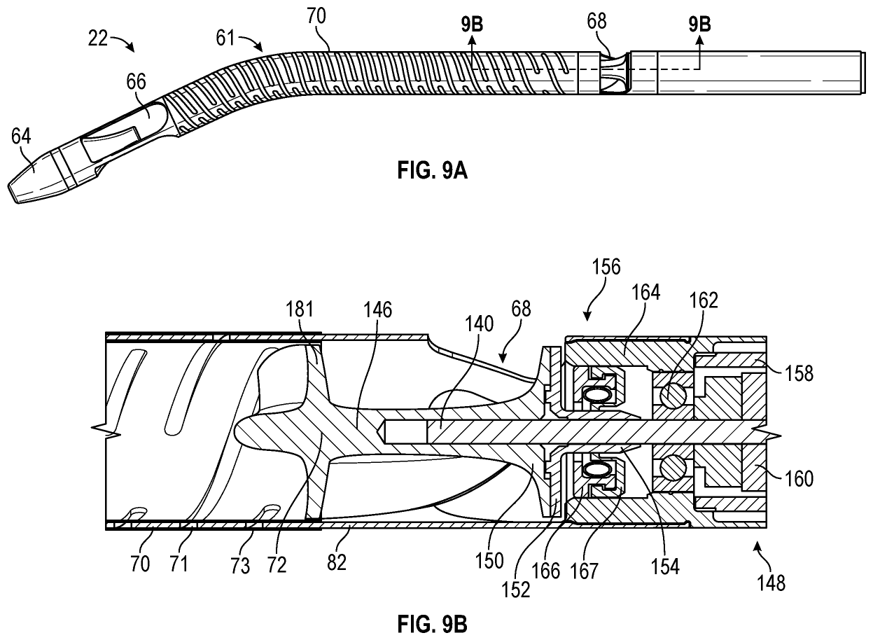

FIGS. 9A and 9B depict side views and a partial cross-section view respectively of the pump 22. As shown, the impeller 72 may be attached to a short, rigid motor drive shaft 140. In the illustrated implementation, the drive shaft 140 extends distally into a proximally facing central lumen in the impeller 72, such as through a proximal extension 154 on the impeller hub 146, where it may be secured by a press fit, laser weld, adhesives or other bonding technique. The impeller 72 may include a radially outwardly extending helical blade 181, which, at its maximum outside diameter, may be spaced apart from the inside surface of tubular impeller housing 82 within the range of from about 40 μm to about 120 μm. Impeller housing 82 may be a proximal extension of the inlet tube 70, on the proximal side of the slots 71 formed in the inlet tube 70 to provide flexibility distal to the impeller. A tubular outer membrane 73 may enclose the inlet tube 70 and seal the slots 71 while preserving flexibility of the inlet tube. Pump outlets 68 may be formed in the sidewall of the impeller housing 82, axially aligned for example with a proximal portion of the impeller 72 (e.g., a proximal 25% to 50% portion of the impeller).

The impeller 72 may comprise a medical grade titanium. This enables a CFD optimized impeller design with minimized shear stress for reduced damage of the blood cells (hemolysis) and a non-constant slope increasing the efficiency. This latter feature cannot be accomplished with a mold-based production method. Electro polishing of the surface of the impeller 72 may decrease the surface roughness to minimize the impact on hemolysis.

In some implementations, the impeller hub 146 flares radially outwardly in a proximal direction to form an impeller base 150, which may direct blood flow out of the outlets 68. A proximal surface of the impeller base 150 may be secured to an impeller back 152, which may be in the form of a radially outwardly extending flange, secured to the motor shaft 140. For this purpose, the impeller back 152 may be provided with a central aperture to receive the motor drive shaft 140 and may be integrally formed with or bonded to a tubular sleeve/proximal extension 154 adapted to be bonded to the motor drive shaft 140. In some implementations, the impeller back 152 is first attached to the motor drive shaft 140 and bonded such as through the use of an adhesive. In a second step, the impeller 72 may be advanced over the shaft and the impeller base 150 bonded to the impeller back 152 such as by laser welding.

The distal opening in the aperture in impeller back 152 may increase in diameter in a distal direction, to facilitate application of an adhesive. The proximal end of tubular sleeve/proximal extension 154 may decrease in outer diameter in a proximal direction to form an entrance ramp for facilitating advancing the sleeve proximally over the motor shaft and through a seal 156, discussed further below.

The motor 148 may include a stator 158 having conductive windings surrounding a cavity which encloses motor armature 160 which may include a plurality of magnets rotationally secured with respect to motor drive shaft 140. The motor drive shaft 140 may extend from the motor 148 through a rotational bearing 162 and also through the seal 156 before exiting the sealed motor housing 164 (also referred to herein as motor housing 74). Seal 156 may include a seal holder 166 which supports an annular seal 167 such as a polymeric seal ring. The seal ring includes a central aperture for receiving the tubular sleeve/proximal extension 154 and is biased radially inwardly against the tubular sleeve/proximal extension 154 to maintain the seal ring in sliding sealing contact with the rotatable tubular sleeve/proximal extension 154. The outside surface of the tubular sleeve/proximal extension 154 may be provided with a smooth surface such as by electro polishing, to minimize wear on the seal.

The pump may include a sealed motor, in applications with a short time of usage for high risk PCI (typically no more than about 6 hours), and be configured for use without flushing or purging. This provides the opportunity to directly bond the impeller 72 on the motor drive shaft 140 as discussed in further detail below, removing issues sometimes associated with magnetic coupling such as the additional stiff length, space requirements or pump efficiency. A four pole motor design enables flow performance up to 4.0 l min−1 (liters per minute) at 60 mmHg with low temperature change. The motor cable interface may be provided with a high tensile strength.

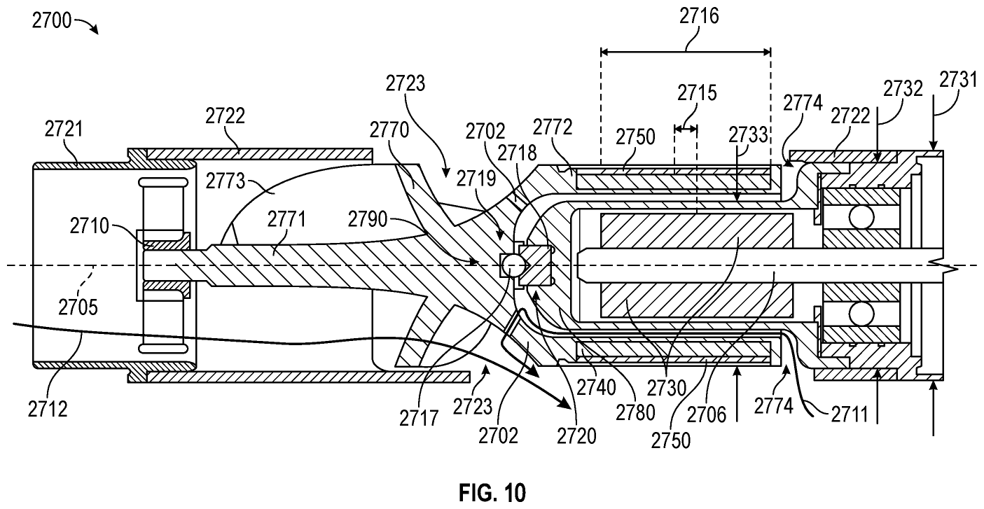

FIG. 10 is a partial cross sectional view, through an impeller and magnetic coupling region, of an embodiment of a rotor bearing system 2700 of a pump that may be used with the various MCS systems described herein. The rotor bearing system 2700 may have a contactless torque transfer, and radial and axial motor mount, that is shown as an exemplary embodiment in the form of a pump for cardiovascular support.

The rotor bearing system 2700 can have a housing 2780. The housing 2780 may be a motor housing that encapsulates a motor, drive shaft and/or a drive magnet, which may be hermetically sealed from the surrounding environment. Within the housing 2780 a first cylindrical permanent magnet 2730 is seated on a shaft 2706 driven by a motor, not shown, and said permanent magnet 2730 is mounted to rotate about a first axis 2705.

The housing 2780 may have a first cylindrical portion having a first outer diameter 2731 (e.g., in a range of 5 to 7 mm, preferably 6 mm) that radially encompasses the motor, a second cylindrical portion having a second outer diameter 2732 that is less than the first outer diameter (e.g., less than the first outer diameter by a range of 0.3 to 1 mm, preferably by 0.5 mm), and a third cylindrical portion having a third outer diameter 2733 that is less than the second outer diameter (e.g., less than the second outer diameter by 1.7 to 2.3 mm, preferably by 2.0 mm).

The second outer diameter 2732 may securely mate with an inlet tube housing 2722, wherein the second outer diameter and the inlet tube housing 2722 are sized so the outer diameter of the inlet tube housing is flush with the first outer diameter 2731 (e.g., the thickness of the inlet tube housing 2722 may be equal to the difference between the first outer diameter and second outer diameter divided by 2. The third outer diameter 2733 of the housing 2780 may be for example in a range of 3.2 to 3.8 mm, preferably 3.5 mm.

The rotor bearing system 2700 may further comprise a rotor 2770 for conveying a liquid, wherein the rotor 2770 comprises a second permanent magnet 2740 in the form of a hollow cylinder that is also mounted to rotate about the first axis 2705. The second permanent magnet 2740 in the form of a hollow cylinder is arranged in a part 2772 in the form of a hollow cylinder of the rotor 2770. The second permanent magnet 2740 in the form of a hollow cylinder optionally comprises a back-iron 2750 on its exterior.

In some embodiments the first permanent magnet 2730 may have an outer diameter of 3 mm, a magnet height of 1 mm, and a length of 3.2 mm (e.g., in a range of 3 to 4.2 mm). The second permanent magnet 2740 may have an outer diameter of 5.3 mm (e.g., in a range of 5 to 5.3 mm), a magnet height of 0.6 mm (e.g., in a range of 0.5 to 0.6 mm), and a length of 3.2 mm (e.g., in a range of 3 to 4.2 mm). The stagger 2715 may be 1 mm (e.g., in a range of 0.1 to 1.2 mm). The rotor 2770 may have an outer diameter of 5.3 mm (e.g., less than the second outer diameter 2732 by a range of 0.1 to 0.4, preferably 0.2 mm) and a length of 15 mm.

The rotor 2770 may be arranged as an impeller that converts the mechanical power transferred by the coupling (e.g., magnetic coupling) into hydraulic power to convey a blood flow against a blood pressure. The rotor 2770 may further comprise a tapered or conical part 2771 that is mated to the part 2772 in the form of a hollow cylinder. The outer circumference of the base surface of the conical part 2771 may be connected with the ring-shaped opening on an axial end of the part 2772 in the form of a hollow cylinder.

The first permanent magnet 2730 and the second permanent magnet 2740 may at least partially axially overlap in the axial area labeled by the reference symbol 2716. The first permanent magnet 2730 is in this case arranged axially staggered in relation to the second permanent magnet 2740. The centers of the first permanent magnet 2730 and the second permanent magnet 2740 are marked by vertical lines, wherein the axial stagger 2715 is drawn between these two vertical lines.

Due to the axial stagger 2715, the second permanent magnet 2740 may be subjected to a force directed to the right in FIG. 10, so that a ball 2717 that is arranged in the rotor 2770 is pushed onto a cone 2718 arranged in the housing 2780, so that a first bearing 2720 and a third bearing 2790, which in this case form a combined axial and radial bearing 2719, are held in contact. Alternatively, a ball may be arranged in the housing 2780 and a cone arranged in the rotor. When used as intended, the ball 2717 rotates in the cone 2718, so that both radial and also axial forces can be absorbed. The combined axial and radial bearing 2719 is in this case a solid body bearing. The ball 2717 is arranged in the conical part 2771. The axial and radial bearing function is achieved by the combination of the two elements ball 2717 and cone 2718. The ball 2717 for example, may have a diameter in a range of 0.5 mm to 0.9 mm, preferably 0.7 mm, and the cone 2718 may have a diameter of 1 mm, a height of 0.8 mm, and a cone angle within a range of 70° to 90°, preferably 80°. The axial bearing function of the combined bearing 2719 has the function of the first bearing and is designed for the relative axial positioning of the rotor 2770 and the housing 2780 and/or the shaft 2706 to each other and to absorb an axial force resulting by the arrangement of the first permanent magnet 2730 and the second permanent magnet 2740. Moreover, the axial force on the rotor bearing system 2700 may be adjusted, so that the exerted force settings can be optimized.

The region of the housing 2780 that comprises the first permanent magnet 2730, may be at least in part radially surrounded by part 2772 in the form of a hollow cylinder of the rotor 2770. A channel 2774 in the form of a hollow cylinder may then be formed between the housing 2780 and part 2772 of the rotor 2770, through which a liquid can flow. Bores or perforations 2702 may be arranged in the rotor 2770, preferably in the conical part 2771 of the rotor 2770, or in a transition of the conical part 2771 to the part 2772 in the form of a hollow cylinder of the rotor 2770 and may be in fluid communication with the channel 2774. In use, when the rotor 2770 spins liquid may be centrifugally expelled from the bores 2702 and liquid may be pulled into the channel 2774 to replace the expelled liquid in a continuous flow. Flow arrow 2711 in this case indicates the direction of flow of the liquid through the gap 2774. Flow arrow 2712 indicates the direction of flow of liquid transferred by the rotor vanes 2773.

A second bearing 2710, which can be arranged as a radial, hydrodynamic, and blood-lubricated plain bearing, may be arranged on the end of the conical part 2771 of the rotor 2770 facing away from the housing 2780. The second bearing 2710 may be designed to absorb radial forces and to position the axis of rotation of the second permanent magnet 2740 in alignment with the axis of rotation 2705 of the shaft 2706 or the first permanent magnet 2730. In this case, the second bearing 2710 may be arranged between the rotor 2770 and an insert 2721, which can be fastened, in particular clamped in or pressed in, in a ring-shaped end on a second housing 2722, which is in turn fastened onto the housing 2780. The second housing 2722 in this case may form an exterior skin of the rotor bearing system 2700, wherein the second housing 2722, which can also be called an impeller housing, has a plurality of outlet windows 2723. The insert 2721 is preferably a bearing housing or star that can be firmly attached (e.g., glued, welded, or friction fitted) to the second housing 2722. The bearing star 2721 may have an outer diameter of 6 mm (e.g., in a range of 5 to 7 mm) and a length of 3 mm (e.g., in a range of 2 to 5 mm). The second housing 2722 may have an outer diameter of 6 mm (e.g., in a range of 5 to 7 mm), a length of 18 mm (e.g., in a range of 15 to 21 mm), and a wall thickness of 0.25 mm (e.g., in a range of 0.15 to 0.5 mm).

Alternatively, the insert 2721 and second housing 2722 may be manufactured as a single piece, which may have a consistent inner diameter. In this arrangement an extended inlet cannula may be connected to the combined insert and second housing 2722 for example by laser welding.

The bearing 2710 may have a diameter of 1 mm (e.g., in a range of 0.75 to 1.5 mm) and a length of 1 mm (e.g., in a range of 0.75 to 2 mm).

Due to the axial stagger 2715 determined by the design between the first permanent magnet 2730 and the second permanent magnet 2740, a defined axial force in the exemplary embodiment in FIG. 10 acts on the rotor 2770 in the direction of the motor, that is to say from left to right in the exemplary embodiment in FIG. 10. This force is opposed by a hydraulic force imposed on the rotor 2770 during operation, that is to say from right to left in the exemplary embodiment in FIG. 10, which is in the opposite direction of liquid flow 2711 generated by the spinning rotor vanes 2773.

In the present case, the axial force originating from the coupling of the first permanent magnet 2730 and the second permanent magnet 2740 may be optimized to be larger than the maximum expected hydraulic force, which ensures that the rotor 2770 is at all times held in a defined axial position, without being too much larger than the maximum expected hydraulic force, which may allow the combined axial and radial bearing 2719 to not be unnecessarily overloaded, thus minimizing friction and wear as well as reduction of torque transmitted to the rotor. This axial force may be optimized by adjusting the dimensions (e.g., length, thickness, outer diameter) of both permanent magnets 2730, 2740, and the axial displacement or stagger distance 2715, and the segment angle, «, if a Halbach configuration is implemented.

Optimization studies were done by the applicant using a Halbach magnet configuration, with a segment angle α of 45° and a pump device having an outer diameter of 6.2 mm. Due to the diameter constraints of the device the inner and outer diameter of the first permanent magnet was chosen to be 1.0 mm and 3.0 mm respectively. The inner and outer diameter of the second permanent magnet was chosen to be 4.1 mm and 5.3 mm respectively. The length of each magnet and the stagger 2715 were modified to study the effect on axial force and torque and optimized. The sum of the magnet length and stagger was limited to 4.2 mm due to a constraint on length of rigid section of the pump so it can traverse tortuous vascular pathway during endovascular delivery to the heart. Conclusions of the study found that an optimized design has a magnet length (length of both permanent magnets 2730, 2740) of 3.2 mm and an axial displacement or stagger 2715 of 1.0 mm generated to best results. A stagger 2715 in a range of 0.5 to 1 mm may be the basis for alternative embodiments but were found to be less optimal. These results may represent an optimized coupling configuration for the devices tested. Because the forces applied to the impeller and coupling are a function of overall device diameter, inlet tube length, impeller design, maximum impeller speed or blood flow rate, and other features or dimensions that affect hydraulic force, bearing frictional losses, and eddy current losses the results may differ with devices having different dimensions or features compared to the ones tested.

For the purposes of this study a maximum fluid load was assumed to be 1.2 mNm, frictional loss of the bearings was assumed to be 0.2 mNm, and eddy current loss was assumed to be 0.1 mNm for a total load torque of 1.5 mNm during normal operation. A safety factor of 3 was used making the maximum load torque 4.5 mNm. The friction and wear behavior can also be optimized by enlarging the cone angle of the cone 2718, wherein sufficient radial load capacity must be ensured.

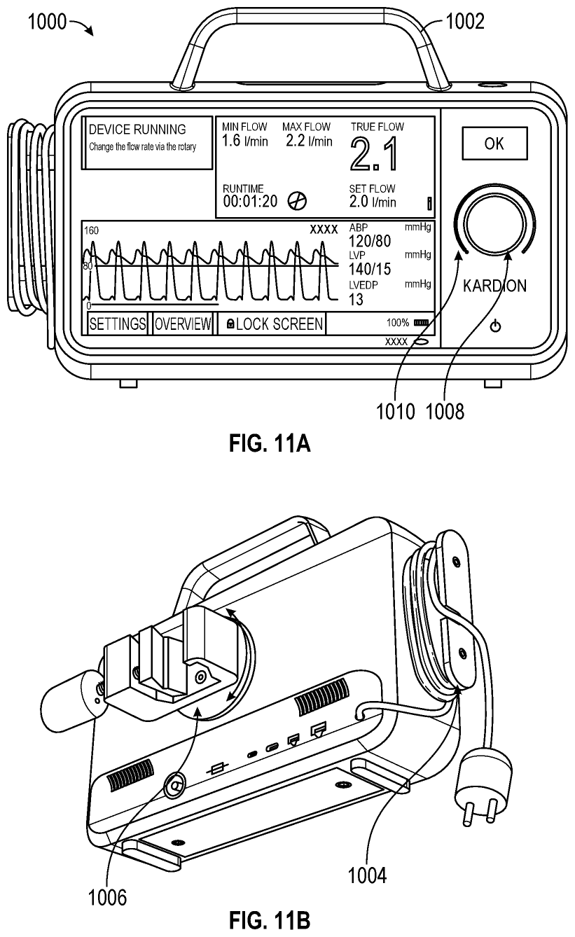

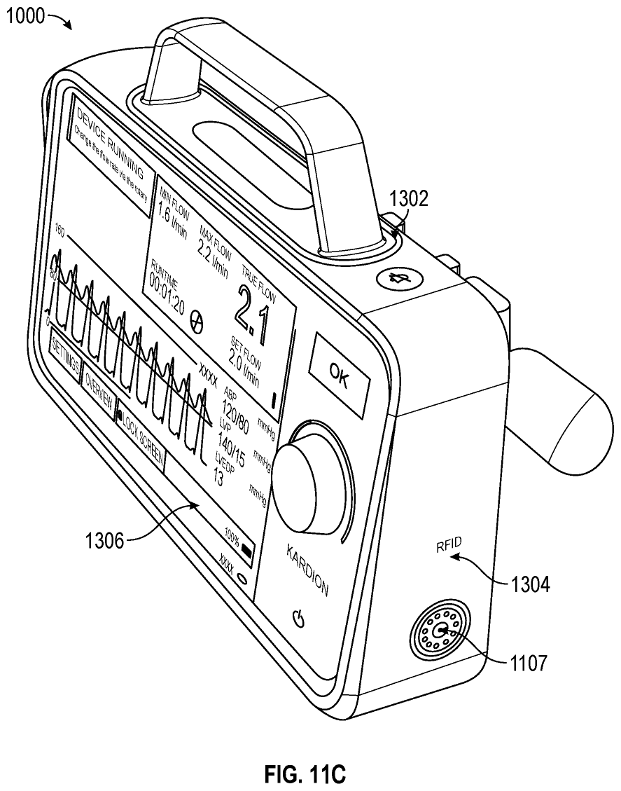

FIGS. 11A-11C show a front view, a back perspective view, and a front perspective view, respectively, of an embodiment of MCS controller or controller 1000. The controller 1000 may support operation of one or more cardiac or circulatory support systems, such as left ventricular support devices, ventricular assist devices, or MCS devices as described herein. The controller 1000 may include one more modules to provide power to the cardiac support systems. The controller 1000 may house electronic circuits to send and receive operational signals to the cardiac support system. The controller 1000 may house one or more hardware processors to receive and process data, such as sensor data, from the cardiac support system. In some embodiments, the controller 1000 may have an integrated or self-contained design in which all or almost all of the components required for operation of the controller are housed within the controller. For example, any power supply components, such as transformers or AC/DC converters, may be housed within the controller 1000. As shown in FIG. 2, the controller 1000 may be wired to the pump 22 via electronic wires extending through the catheter shaft 16 to the pump 22.

In some embodiments, the controller 1000 may include communications systems, or any other suitable systems, to allow the controller 1000 to be adapted to new or modified uses after construction of the controller. For example, multiple modes of wired or wireless communication can be integrated within the controller 1000 to communicate with outside technology, such as, for example, RF, wifi, and/or Bluetooth. In some embodiments, the controller 1000 may have an RFID reader. In some embodiments, the controller 1000 may have systems or components that enable syncing patient data, telemedicine, patient monitoring, real time data collection, error reporting, and/or sharing maintenance records.

The controller 1000 may include a housing for these modules that support any of the cardiac support systems described herein. The housing may further include a handle 1002 to support portability. In contrast to some of the other controllers, such as Abiomed's Impella Controller, the controller 1000 may not include components required for purging. For example, the controller 1000 does not include a cassette for purging. The cassette typically delivers rinsing fluid to the catheter. However, the cassette requires significant real estate and makes the housing bigger and heavier. Due to the design improvements described herein, such as bearing design and sealed motor discussed herein, the controller 1000 does not include a cassette. Furthermore, in some embodiments, the controller 1000 does not require a port for receiving a purging tube. Accordingly, the controller 1000 may be light and compact to support portability.

The controller may also include a cable management support 1004. In some embodiments, the cable management support 1004 is positioned on one end or side of the controller 1000. The controller 1000 may also include a mount 1006 that may support mounting the controller to a pole in a clinical environment. The mount 1006 may rotate about an axis to support horizontal or vertical clamping. The mount 1006 may be rapidly locked into the desired orientation by quick fastening with a slipping clutch. In some instances, the mount 1006 is positioned away from the cable management support 1004. Furthermore, in some embodiments, the cable management support 1004 is positioned on a left end of the controller 1000 as shown in FIG. 11B. The port 1107 (such as shown in FIG. 11C) can be positioned on a side opposite from the cable management support 1004. In some instances, the control element 1008 discussed below is positioned on a side opposite from the cable management support 1004 and in close proximity to the port 1107. This may enable a user to have an improved interaction with the active components of the controller 1000. Therefore, the arrangement of all these elements in the controller 1000 as illustrated can improve operational experience and improve portability.

The controller 1000 may include a control element 1008. In some embodiment, the control element 1008 may provide a haptic feedback. The control element 1008 can include a push button rotary dial. The control element 1008 may enable a user to change parameters on the controller 1000 to control one or more processes described herein. The control element 1008 may also include status indicator 1010 as illustrated in FIG. 11A. In some embodiments, the controller 1000 may include a separate confirmation control element. Furthermore, in some embodiments, aside from the separate confirmation control element, all the parameters can be modified using a single control element 1008. The grouping of controls in a dedicated area may improve user experience.

In some embodiments, the controller 1000 can include an alarm feedback system, which can provide feedback to an operator regarding the operation of the MCS system. In some embodiments, the alarm feedback system can be in the form of an LED 1302 as illustrated. The LED 1302 may be positioned so that it can be seen by an operator using the controller. As illustrated, the LED 1302 is positioned around the handle 1002. Therefore, it can be seen from positions 360° around the controller. The LED 1302 may be in the form of a ring (oval, oblong, circular, or any other suitable shape) wrapping the handle 1002. Such an LED 1302 may be visualized from any direction as long as the top of the controller is viewable. A control system can generate different colors or patterns for the LED 1302 to provide various alarms or status of the controller 1000 and/or an MCS device.

The controller 1000 further includes a port 1107 that may receive a cable connected to an MCS device 22. The port 1107 can support multiple versions of MCS devices. The controller 1000 can also include an RFID reader 1304 on a side of the controller 1000. The RFID reader 1304 can read badges of a sales representative and operate the device according to a particular demo mode. The controller 1000 can include a glass cover 1306 that is tilted as shown in FIG. 11C to improve readability for the user.

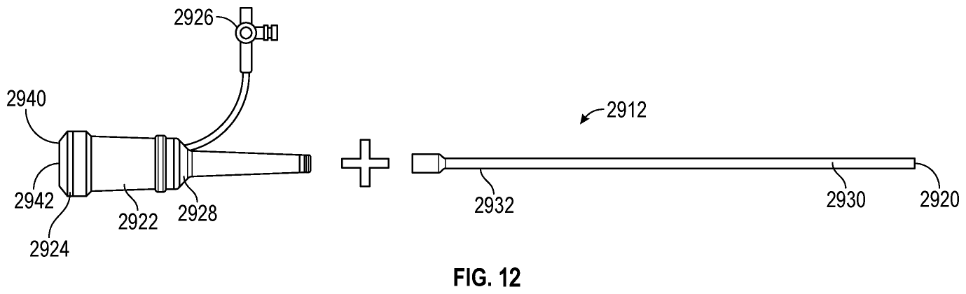

FIG. 12 illustrates a side elevational view of an expandable introducer sheath 2912. The expandable introducer sheath 2912 may be used with any of the embodiments of the MCS system or pump described herein. The expandable introducer sheath 2912 may have a hub 2922 and associated components similar to the introducer sheath 112 described in connection with FIG. 5, and vice versa. Further, the elongate tubular body of the introducer sheath 2912 may be expandable from a first reduced inside cross-sectional area to a second, enlarged inside cross-sectional area, such as to permit passage of a device having an outer diameter (OD) greater than the first reduced cross-sectional area. The introducer sheath may be biased to return to or approximately to the first reduced cross-sectional area following expansion in response to passage of a sheath enlarging device therethrough (e.g., the MCS devices described herein). The expandable introducer sheath 2912 may include an expandable support structure 2932 such as a tubular framework of a plurality of zigzag segments of a shape memory material such as Nitinol which permit radial expansion in the presence of an enlarging device passing therethrough, but will return to the first reduced cross-sectional area following removal and/or passing of the device. The expandable support structure 2932 may be enclosed within a tubular flexible membrane 2930, which can accommodate radial expansion and contraction. Further as shown, the expandable introducer sheath 2912 may include a distal end 2920, a proximal end 2940, a side port 2926, suture eyelet(s)/eye 2928, a proximal hub 2922, and a proximal end port 2942 similar to the distal end 120, proximal end 118, side port 126, suture eyelet(s)/eye 128, proximal hub 122, and proximal end port 124 of the introducer sheath 112 described herein. The expandable introducer sheath 2912 may also include a locking cap 2924 at its proximal end with one or more features that can engage/lock with an insertion tool and/or an insertion catheter (e.g., insertion tool 32, insertion tool 2632, insertion catheter 2832, and/or any of the insertion tools or catheters described herein) such as a connector 2639 and/or a dilator (e.g., dilator 114) as described herein.

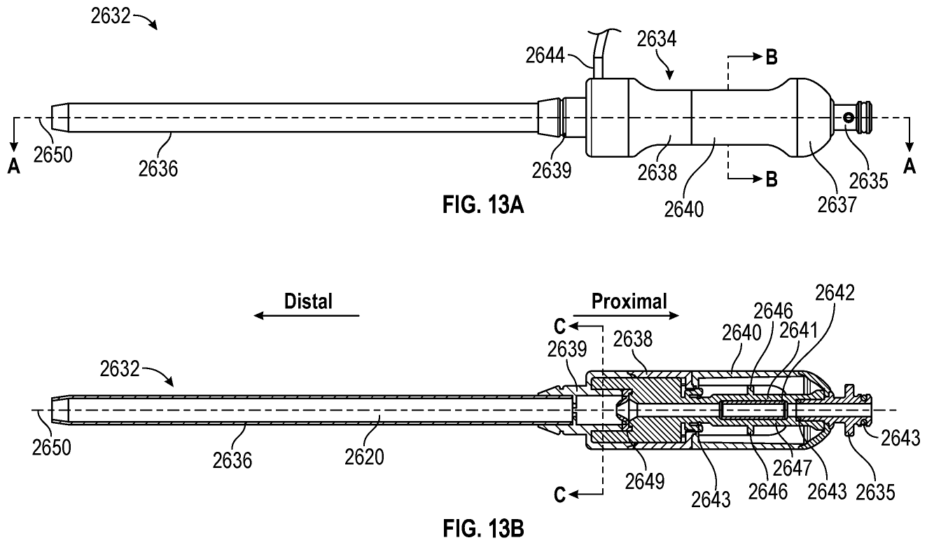

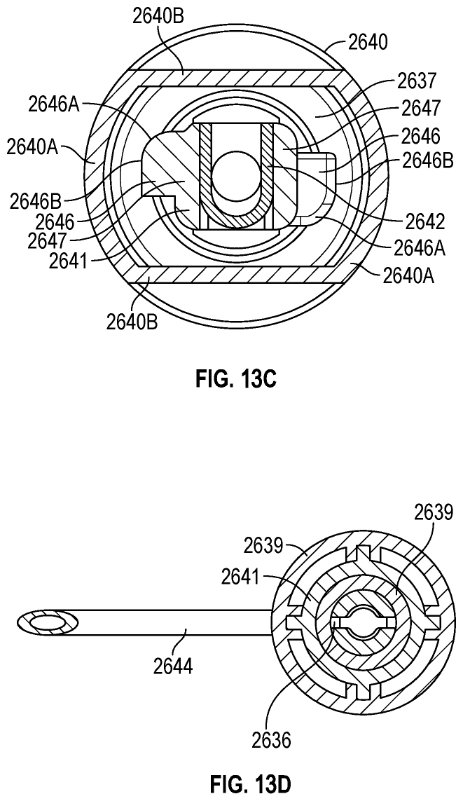

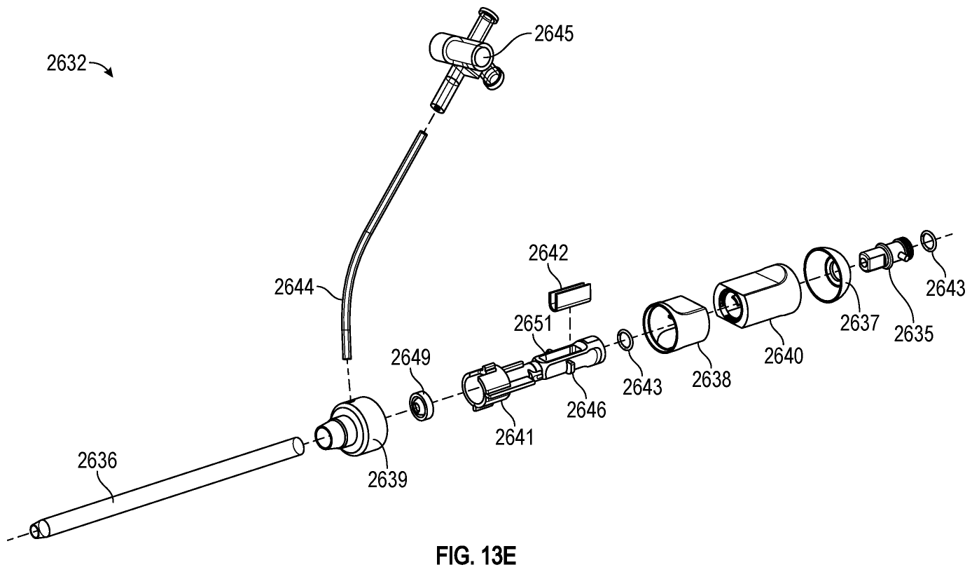

FIG. 13A-13E are various views of an embodiment of an insertion tool 2632. FIG. 13A is a side view of the insertion tool 2632, FIG. 13B is a longitudinal cross-section view of the insertion tool 2632 as taken along the line A-A in FIG. 13A, FIGS. 13C and 13D are cross-section views as taken along the lines B-B and C-C respectively as indicated in FIGS. 13A and 13B, and FIG. 13E is an exploded view of the insertion tool 2632. The insertion tool 2632 may have the same or similar features and/or functions as the insertion tool 32 of FIG. 4, and vice versa. Thus, the insertion tool 2632 may be used with the pump 22, or any other pump described herein, etc.

The insertion tool 2632 may have a generally elongate tubular configuration defining a longitudinal axis 2650. As shown in FIG. 13A, the insertion tool 2632 may comprise a tubular body 2636, which may be a cylindrical tube, at a distal end. The insertion tool 2632 may comprise a hub 2634 at a proximal end. The hub 2634 may include a connector 2639 (also referred to herein as a first engagement structure), a first housing section 2638, a second housing section 2640, a cap 2637, and/or a plug 2635. The connector 2639 may include tubing 2644 with a valve 2645 (shown in FIG. 13E). As further shown in the cross sectional view of FIG. 13B, the insertion tool 2632 may also include a locking mechanism 2641, a locking pad 2642, a hemostatic valve 2649, and/or one or more sealing elements 2643. The locking mechanism 2641 may comprise locking tabs 2646 as further described below.

The tubular body 2636 at the distal end of the insertion tool 2632 may have a distal end and a proximal end with a lumen extending therebetween. The tubular body 2636 may be cylindrical. The tubular body 2636 may be made of polymer, plastic, other suitable materials, or combinations thereof. The tubular body 2636 may be made of a transparent polymer such as nylon, Grilamid®, Pebax®, which may facilitate visual confirmation of the passage of a guidewire 100 through a guidewire guide tube 83 contained in the tubular body 2636. The tubular body 2636 may be expandable. The distal end of the tubular body 2636 may comprise a taper, for example a conical portion that reduces in diameter in the distal direction, to facilitate insertion of the insertion tool 2632 (such as insertion into an introducer sheath as described herein). The distal end of the tubular body 2636, such as the tapered distal end, may removably fit into the proximal opening 90 of the guidewire aid 38. The tapered end may be a material such as 55D Pebax® molded to the tubular body. The tubular body 2636 may connect at its proximal end to a distal end of the connector 2639. The connector 2639 may connect at its proximal end to a distal end of the first housing section 2638. The first housing section 2638 may connect (e.g., rotatably, rotatable between an open position and a locked position which may be switched back and forth by rotating the second housing section 90 degrees with respect to the first housing section) at its proximal end to a distal end of the second housing section 2640. The second housing section 2640 may connect at its proximal end to a distal end of the cap 2637. A distal end of the plug 2635 may connect through a proximal end of the cap 2637.

The locking mechanism 2641 may have a longitudinally extending lumen through its body with a recess 2651 configured to accept the locking pad 2642. The locking pad 2642 may be an elastomeric material with soft durometer such as a thermoplastic elastomer, soft Pebax® or silicone. When inserted in the recess 2651, the locking pad 2642 may have an inner surface that substantially coincides with an inner surface of the longitudinally extending lumen of the locking mechanism 2641. As shown in FIG. 13B, the locking mechanism 2641 may be disposed within the hub 2634 comprising the connector 2639, the first housing section 2638, the second housing section 2640, and the cap 2637, such that all share the common longitudinal axis 2650 and the lumen of the locking mechanism 2641 is concentric with the lumen of the tubular body 2636 at least in the unlocked configuration. The locking mechanism 2641 may connect at its distal end to the proximal end of the connector 2639, and may connect at its proximal end to the distal end of the plug 2635. The plug 2635 may have a longitudinally extending lumen through its body from its distal end to its proximal end.

When connected, the plug 2635, the locking mechanism 2641, the connector 2639, and the tubular body 2636 may create a fluidically sealed pathway extending along the longitudinal axis 2650 of the insertion tool 2632. The pathway may be fluidly sealed with the pump and catheter shaft inserted therein. The valve 2649, and/or one or more sealing elements 2643 such as O-rings, may aid in creating the fluidically scaled pathway. For example, the connection between the proximal end of the connector 2639 and distal end of the locking mechanism 2641 may comprise the valve 2649. The valve 2649 may have a conical flap that reduces in width in the distal direction. When the pump or catheter shaft are inserted through the valve 2649, the conical sidewalls may expand to allow the components therethrough but stay compressed about the components to create the seal. The connection between the proximal end of the locking mechanism 2641 and the distal end of the plug 2635 may comprise one of the sealing elements 2643. The proximal end of the plug 2635 may comprise one of the sealing elements 2643 to fluidly connect to other components of the circulatory support system, such as a distal connector of the sterile sleeve 26, which may have a mating feature that locks to the plug 2635 such as by rotating projections on the plug into slots in the mating feature. The sealing elements 2643 may be O-rings or other rounded sealing elements that may sealingly engage components passing therethrough.

The fluidically sealed pathway along the longitudinal axis 2650 of the insertion tool 2632 may be configured to axially movably receive a circulatory support device or pump, such as any of the devices or pumps described herein. For example, the lumen 2620 of the tubular body 2636 may be configured to axially movably receive the pump 22 and optionally a guidewire guide tube 83, and the longitudinally extending lumen in the hub 2634 may be sized to slidably receive the shaft 16 of the MCS device (e.g., an 8 French shaft). When the pump 22 is contained in the lumen of the tubular body 2636, the shaft 16 is contained in the longitudinally extending lumen in the hub 2634, and the locking mechanism is in an unlocked state (as shown in FIG. 13C), the pump 22 may be advanced distally out of the tubular body 2636, for example into the tubular body 116 of the introducer sheath 112 before advancing from the introducer sheath into the patient's vasculature by advancing the shaft 16 in a distal direction. The tubular body 2636 of the insertion tool 2632, with a circulatory support device such as the pump 22 therein, may be configured to be received by an introducer sheath (e.g., introducer sheath 112 and/or 2912) as described herein. As such, the tubular body 2636 of the insertion tool 2632 may have sufficient collapse resistance to maintain patency when passed through the hemostatic valves of the introducer sheath.

The insertion tool 2632 may be configured to releasably lock with the circulatory support device when inserted into the insertion tool 2632. In some embodiments, the insertion tool 2632 may releasably lock with the MSC shaft 16 (also referred to as catheter or catheter shaft) of the circulatory support device. When the insertion tool 2632 is locked with the circulatory support device, axial (e.g., longitudinal/proximal/distal) movement and/or rotational movement of the circulatory support device may be prevented. The insertion tool 2632 may lock to the circulatory support device by engagement of the locking pad 2642 with at least a portion of the circulatory support device. To engage the locking pad 2642 with the at least a portion of the circulatory support device such as the shaft 16, the locking pad 2642 may be compressed by the locking mechanism 2641.

The locking mechanism 2641 may compress the locking pad 2642 by interaction between one or more locking tabs 2646 of the locking mechanism 2641 and an inner surface or surfaces of the second housing section 2640. The locking tabs 2646 may extend radially outwardly from opposing sidewalls 2647 of the locking mechanism. The locking tabs 2646 may be offset along the longitudinal axis 2650. The second housing section 2640 along with the cap 2643 may be configured to rotate relative to the first housing section 2638, the locking tabs 2646, and the plug 2635 (with an axis of rotation being along the longitudinal axis 2650 of the insertion tool 2632). Configured this way, when the second housing section 2640 is rotated, one or more inner surfaces or sidewalls 2640B of the second housing section 2640 may contact one or more of the locking tabs 2646, causing the locking tabs 2646 to compress inwards leading to radially inward compression of the locking pad 2642. As shown in FIG. 13C, if the second housing section 2640 is rotated 90 degrees counterclockwise (as oriented in FIG. 13C, or clockwise with respect to the first housing section 2638), the inner surface sidewalls 2640B of the second housing section 2640 may contact the locking tabs 2646 (which in this embodiment are shown to have a curved outer surface) and force them inward, compressing the locking mechanism 2641 inward against the locking pad 2642. In cases where the locking tabs 2646 are offset longitudinally, the inward compression of the locking tabs 2646 and thus the locking pad 2642 against, for example, the shaft 16 may cause the shaft 16 to bend slightly in the region of the locking pad 2642, holding the shaft 16 in place. Alternatively or in addition, the shaft 16 may be compressed by the locking pad 2642 and hold/lock the shaft 16 in place.

As shown in FIG. 13C, the second housing section 2640 may include two opposing first sidewalls 2640A, which may be rounded as shown, connected by two opposing second sidewalls 2640B, which may be straight. A first distance between the two opposing first sidewalls 2640A, for example a first diameter, may be greater than a second distance between the two opposing second sidewalls 2640B, for example a second diameter. In the unlocked state, as shown in FIG. 13C, the two opposing first sidewalls 2640A may be adjacent respective locking tabs 2646. When rotated into the locked position, the two opposing second sidewalls 2640B may contact and compress respective locking tabs 2646, as described, due to the shorter distance between the second sidewalls 2640B. The locking tabs 2646 may each comprise a rounded outer corner 2646A that is contacted by a respective second sidewall 2640B, for a gradual compression and to reduce the risk of breaking the tabs. As the second housing section 2640 is turned farther counterclockwise as oriented (i.e., clockwise with respect to the first housing section 2638, the locking tabs 2646 may each comprise radially outer edges 2646B that are contacted by a respective second sidewall 2640B. The edges 2646B may be straight as shown, or otherwise match the contour of the inner surface of the second sidewall 2640B. With two opposing straight surfaces, for example, of the edges 2646B and the second sidewall 2640B in contact, the second housing section 2640 may be rotationally stationary without needing an external force by a user. Movement into engagement of the edges 2646B with the inner surface of the second sidewall 2640B may create a snap-like haptic feedback.

To unlock the circulatory support device from the insertion tool 2632, the second housing section 2640 may be rotated in the opposite direction (clockwise as oriented in FIG. 13C, or counterclockwise with respect to the first housing section 2638). The first housing section 2638 and the second housing section 2640 may comprise features that can keep the insertion tool 2632 in the unlocked position until a user of the system chooses to lock the circulatory support device in place relative to the insertion tool 2632. In some embodiments, interaction between the locking tabs 2646 and the second housing section 2640 may keep the insertion tool 2632 in the unlocked position until a user of the system chooses to lock the circulatory support device relative to the insertion tool 2632. Likewise, the first housing section 2638 and the second housing section 2640 may comprise features that can keep the insertion tool 2632 in the locked position, as described, until a user of the system chooses to unlock the circulatory support device relative to the insertion tool 2632. In some embodiments, the interaction between the locking tabs 2646 and the second housing section 2640 may keep the insertion tool 2632 in the locked position until a user of the system chooses to unlock the circulatory support device relative to the insertion tool 2632.

The connector 2639 of the insertion tool 2632 may be configured to engage with (e.g., releasably lock/unlock with) an introducer sheath as described herein. For example, the outer surface of the distal end of the connector 2639 may comprise an inward circumferential groove that can be used to engage with a component such as mating bumps or flexible tabs in a locking cap 2924 in a proximal end port 2942 of the introducer sheath hub and/or lock of the introducer sheath. Engagement of the distal end of the connector 2639 with the locking cap 2924 may create a snap-like haptic feedback. The connector 2639 may mate with the introducer sheath locking cap 2924 in a manner that prevents rotation of the insertion tool connector 2639 with respect to the introducer hub 2922, preventing rotation of the first housing section 2638 with respect to the introducer hub when connected. For example, the distal end of the connector 2639 and the proximal end port 2942 of the introducer sheath may be oval, oblong, square, polygonal, or a non-circular in cross-sectional shape. Furthermore, the cross-sectional shape of the connector 2639 can taper to a smaller cross-sectional area in the distal direction to facilitate insertion into the locking cap 2924 of the introducer sheath. Having the insertion tool 2632 lock with respect to the introducer sheath can facilitate handling by allowing a user to hold the introducer hub 2922 and/or the first housing section 2638 with one hand while rotating the second housing section 2640 with the other hand.

FIG. 13D shows part of the connections between the connector 2639 and a distal end of the locking mechanism 2641, and the connector 2639 and a proximal end of the elongate tubular body 2636. Also shown is the tubing 2644 that may, in some embodiments, fluidly connect to the longitudinal lumen of the insertion tool 2632. The locking mechanism 2641 may include projections extending radially outwardly that are received into corresponding grooves or recesses of the connector 2639. This engagement may rotationally stabilize the locking mechanism 2641 with respect to the connector 2639. Adhesive may be added to adhere the projections and the grooves to firmly connect the connector 2639 and the locking mechanism 2641. Adhesive may be added to adhere the locking mechanism 2641 to the first housing section 2638 to firmly connect them as well. The connector 2639 may have an inward flange that has a lumen having the same size and sharing the axis 2650 with the longitudinally extending lumen in the hub 2634, which may provide a stop when inserting the tubular body 2636 into the connector 2639 during manufacturing which may protect the valve 2649 and keep the opening to the tube 2644 patent.

FIG. 13E shows an exploded view of the insertion tool 2632 according to FIGS. 13A-D and to some embodiments. As shown, the tube 2644 that may be fluidly connected to the longitudinal lumen of the insertion tool 2632 and may have a valve 2645, such as a stopcock, at its opposite end. The valve 2645 may be adjusted to prevent or allow fluid flow through the valve 2645.

The insertion tool 2632 may have a length within the range of from about 85 mm to about 200 mm (e.g., about 192 mm). In some embodiments, the longitudinal lumen of the insertion tool 2632 may comprise a diameter within the range of from about 4.5 mm to about 8.0 mm (e.g., about 5.55 mm). The insertion tool 2632 may be sized and configured such that the marking 37 (see FIG. 7) is revealed proximal to the insertion tool hub 2634 when the pump 22 is fully within the tubular body 2636. The insertion tool 2632 may include a hemostasis valve (e.g., hemostatis valve 2645) to seal around the circulatory support system passing therethrough (e.g., to seal around the MCS shaft 16). If provided, the hemostasis valve may accommodate passage of the larger diameter MCS device which includes the pump. In a commercial embodiment of the circulatory support system, the MCS device as packaged is pre-positioned within the insertion tool 2632 and a guidewire aid is pre-loaded within the MCS device and shaft 16, as described herein.

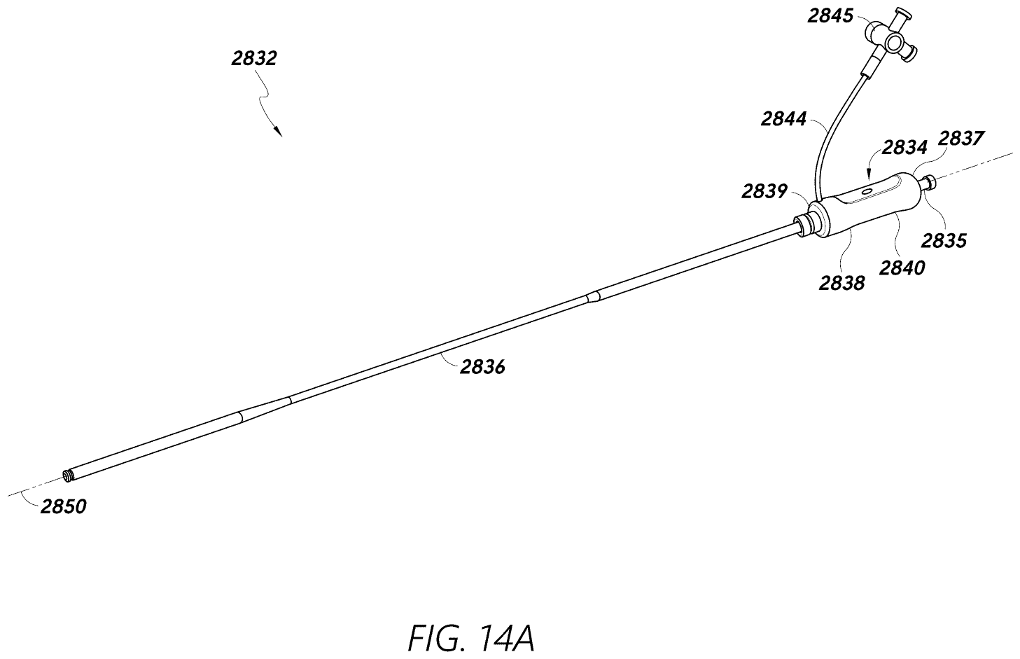

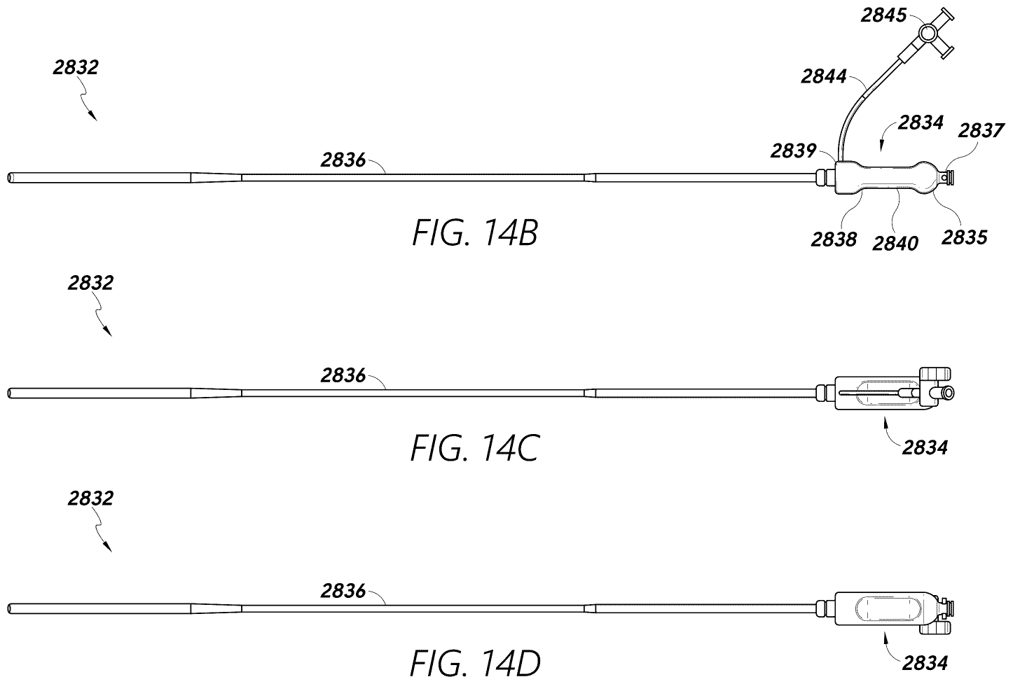

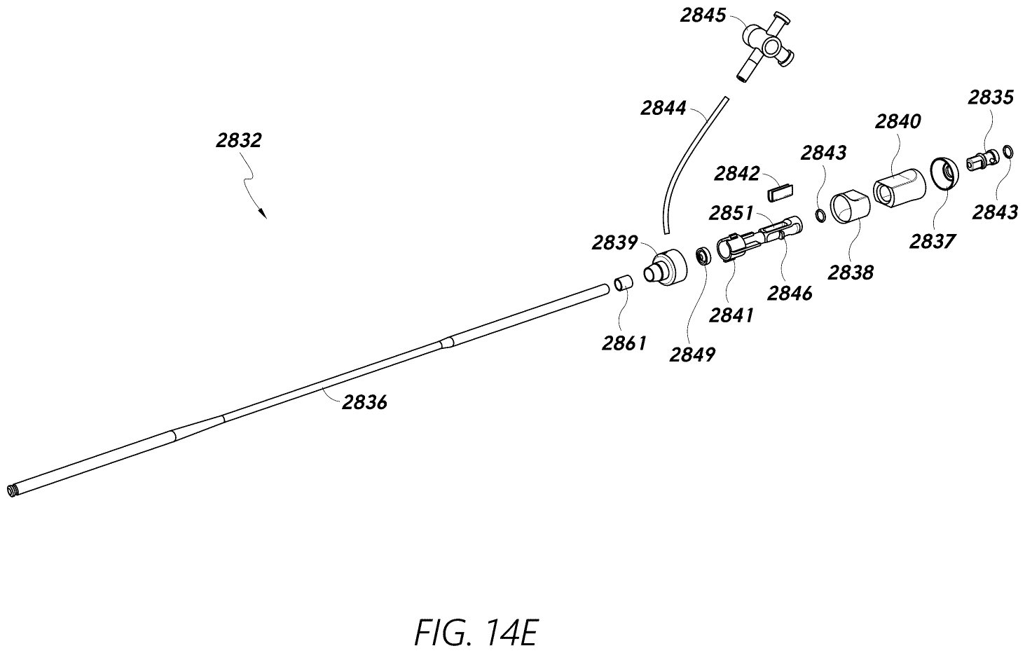

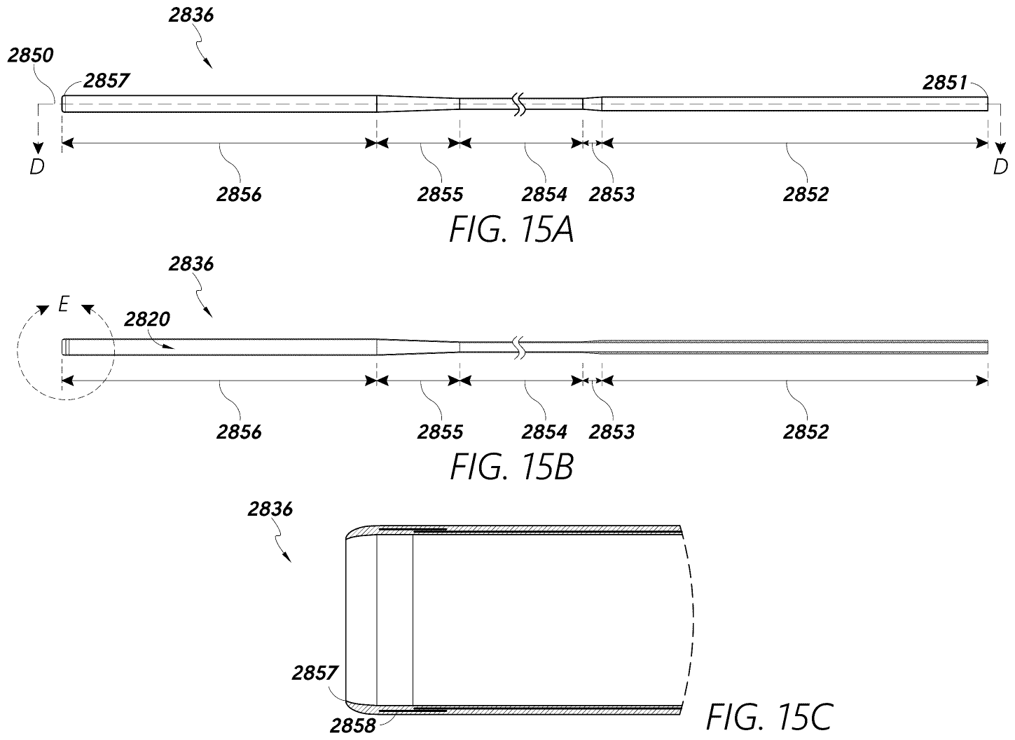

FIGS. 14A-14E are various views of an embodiment of an insertion catheter 2832 that may be used with the various MCS systems and devices described herein. FIG. 14A is a perspective view, FIG. 14B a side view, FIG. 14C a top view, FIG. 14D a bottom view, and FIG. 14E an exploded perspective view of the insertion catheter 2832. The insertion catheter 2832 can have the same or similar features and/or functions as the insertion tools described herein, such as insertion tools 32 and 2632. For instance, at least some features of the insertion catheter 2832 that can be the same or similar as those of the insertion tool 2632 are numbered with the same last two digits but begin with “28” instead of “26.” For example and as shown in FIGS. 14A-14E, the insertion catheter 2832 can have a generally elongate tubular configuration having a proximal end and a distal end with a longitudinal axis 2850 extending therebetween, a tubular body 2836 aligned with the longitudinal axis 2850, and a hub 2834 connected to the tubular body 2836. The hub 2834 can include the same or similar features as the hub 2634, including having a locking mechanism 2841 with recess 2851, locking tabs 2846 and locking pad 2842 that can be the same or similar and/or function the same or similar to the locking mechanism 2641 with recess 2651, locking tabs 2646 and locking pad 2642. The hub 2834 may include a connector 2839, a first housing section 2838, a second housing section 2840, a cap 2837, a plug 2835, a hemostatic valve 2849, and/or one or more sealing elements 2843. The connector 2839 may include tubing 2844 with a valve 2845. The insertion catheter 2832 can also include other features that are the same or similar to the insertion tool 2632, however the tubular body 2836 can be configured differently than the tubular body 2636 as will be discussed with respect to FIGS. 15A-15C. Furthermore, with a different tubular body 2836, the insertion catheter 2832 can include an adapter ring 2861 as shown in FIG. 14E configured to fluidically connect and seal the tubular body 2836 with the hub 2834 (e.g., with the connector 2839 of the hub 2834).

FIGS. 15A-15C are various views of the tubular body 2836 of the insertion catheter 2832. FIG. 15A is a side view of the tubular body 2836, FIG. 15B is a longitudinal cross-section view of the tubular body 2836 as taken along the line D-D in FIG. 15A, and FIG. 15C is a magnified view of detail E of FIG. 15B. The tubular body 2836 can comprise one or more features that advantageously increase the operability and/or case of use of the insertion catheter 2832, as will be described further below.

The tubular body 2836 can have a proximal end 2851 and a distal end 2857 with the longitudinal axis 2850 extending therebetween, a proximal portion 2852 adjacent the proximal end 2851, a distal portion 2856 adjacent the distal end 2857, and a mid portion 2854 between the proximal portion 2852 and the distal portion 2856. Different than the tubular bodies of other insertion tools described herein, the distal, mid, and proximal portions can each have different inner and/or outer diameters than one another, thus the tubular body 2836 can also include a proximal transition section 2853 between the proximal portion 2852 and the mid portion 2854, and a distal transition section 2855 between the mid portion 2854 and the distal portion 2856. All portions and sections of the tubular body 2836 can be concentric about the longitudinal axis 2850 as shown. Furthermore, all portions and sections of the tubular body 2836 can together form a common lumen 2820.

The tubular body 2836 can be configured to extend past a distal end of an introducer sheath (e.g., any of the introducer sheaths described herein) when the insertion catheter 2832 is in use. As such, the tubular body 2836 of the insertion catheter 2832 can have a length within a range of from about 275 mm to about 675 mm, an inside diameter within a range of from about 1.5 mm to about 6 mm, and an outside diameter within a range of from about 2 mm to about 7 mm. The distal portion 2856 and the distal transition section 2855 can be configured to receive (e.g., axially movably receive) at least a portion of the circulatory support device 22 and optionally a guidewire guide tube 83 connected thereto (for example, wherein the guidewire guide tube is at least partially positioned external to the circulatory support device). As such, the distal portion 2856 can have a length within a range of from about 75 mm to about 140 mm, or a length configured to contain up to 100% of the MCS device, or a length configured to contain the MCS device with only the tapered portion of the distal tip 64 protruding from the distal end 2857, and an inside diameter within a range of from about 3.5 mm to about 6 mm, or from a first inner diameter configured to slidably contain the catheter shaft 16 of the MCS device 22 to a second inner diameter configured to slidably contain the MCS device 22. The distal transition section 2855 can have a length within a range of from about 5 mm to about 55 mm and an inside diameter and an outside diameter that vary along its length to meet at its ends the inside and outside diameters of the distal and mid portions. The mid portion 2854 and the proximal portion 2852 can be configured to receive (e.g., axially movably receive) the elongate flexible catheter shaft 16 that extends proximally from the circulatory support device 22. As such, the mid portion 2854 can have a length within a range of from about 150 mm to about 250 mm and an inside diameter within a range of from about 1.5 mm to about 4.5 mm. The proximal portion 2852 can have a length within a range of from about 100 mm to about 165 mm and an inside diameter within a range of from about 2.0 mm to about 6 mm. The proximal transition section 2853 can have a length within a range of from about 1 mm to about 20 mm and an inside and an outside diameter that vary along its length to meet at its ends the inside and outside diameters of the mid and proximal portions. Overall and as shown, the distal portion 2856 can have a larger inner diameter than an inner diameter of the mid portion 2854 and an inner diameter of the proximal portion 2852. Furthermore, the mid portion 2854 can have a smaller outer diameter than an outer diameter of the proximal portion 2852 and an outer diameter of the distal portion 2856. In some embodiments, the outer diameter of the proximal portion 2852 can be the same or similar to the outer diameter of the mid portion 2854, and as such no proximal transition section 2853 may be required.

The tubular body 2836 can be a composite body having a liner and a jacket material, such as Pebax 72D with Propell. Different than the tubular bodies of other insertion tools described herein, the tubular body 2836 can vary in stiffness and/or its ability to resist kinking along its length. As shown in the cross-section of FIG. 15B, the proximal portion 2852 can be configured to have a greater stiffness than portions of the tubular body 2836 distal to the proximal portion so as to transmit longitudinal force without kinking. To attain such greater stiffness, the proximal portion 2852 can have a greater wall thickness than other portions of the tubular body 2836, a larger diameter (e.g., a larger outer diameter) than other portions of the tubular body 2836, and/or a reinforced wall structure. For example, the proximal portion 2852 and optionally the proximal transition section 2853 can include a reinforced double-braid wire such as 10 PPI, 0.002″×0.005″ SS 304V wire. Conversely, other portions and/or sections of the tubular body 2836, such as the distal portion 2856 and optionally the distal transition section 2855, can include a braided wire (e.g., a single-braided wire) such as 38 PPI, 0.002″×0.005″ SS 304V wire.

Similar to the insertion tool 2632, the distal end 2857 of the tubular body 2836 can have a taper, for example a conical portion that reduces in diameter in the distal direction, to facilitate insertion of the insertion catheter 2832 (such as insertion into an introducer sheath as described herein). The distal end of the tubular body 2836, such as the tapered distal end, may removably fit into the proximal opening 90 of the guidewire aid 38. Additionally, the tubular body 2836 can include a band 2858 adjacent its distal end 2857. The band 2858 can be a PET band configured to hold ends of the braided wire of the distal portion 2856. In alternative embodiments, the band 2858 can be a radiopaque marker.

To facilitate insertion of the insertion catheter 2836 (such as insertion into an introducer sheath as described herein), at least the distal portion 2856 of the tubular body 2836 can have a hydrophilic coating to reduce friction on an exterior surface thereof. In some embodiments, more than the distal portion or the entirety of the tubular body 2836 can be coated with a hydrophilic coating to reduce external surface friction. Further related to insertion, the tubular body 2836 of the insertion catheter 2832 can be configured to have sufficient collapse resistance to maintain patency when passed through one or more hemostatic valves of an introducer sheath.

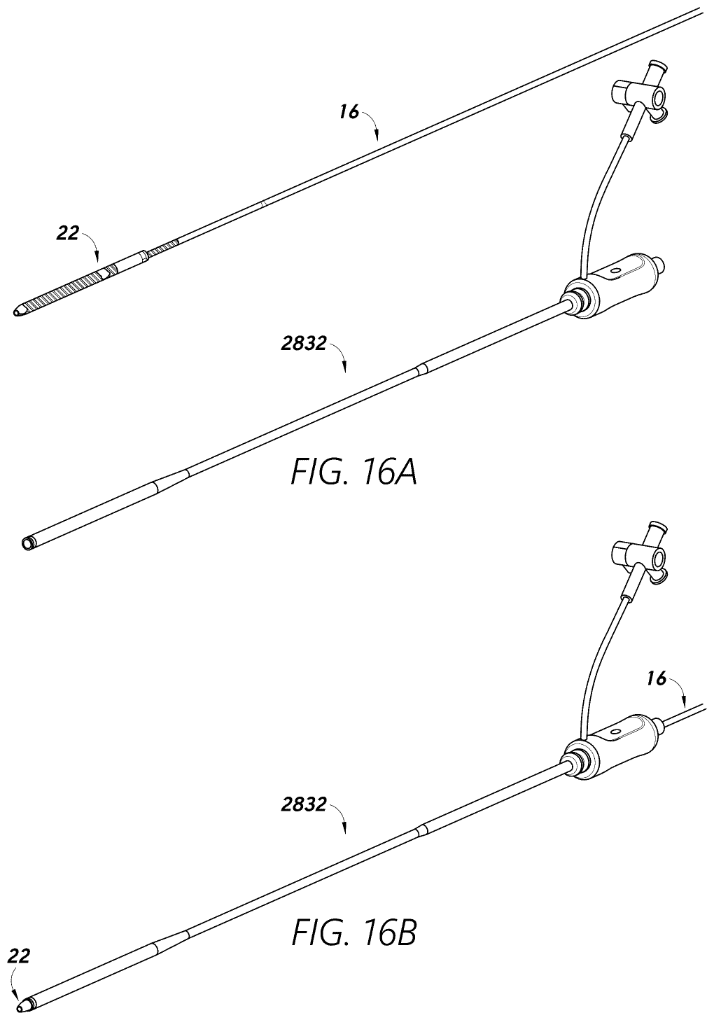

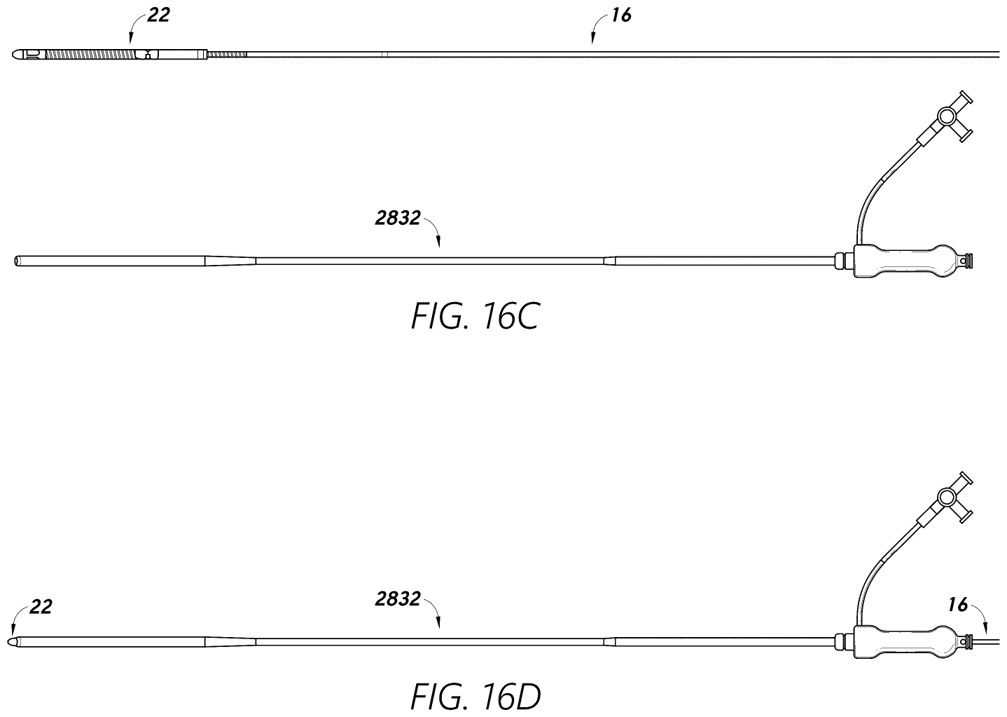

FIGS. 16A-16D are various views of the MCS device 22 and its catheter shaft 16 separate of and placed within the insertion catheter 2832. FIG. 16A shows a perspective view of the MCS device 22 and its catheter shaft 16 at top separate of the insertion catheter 2832 at bottom, whereas FIG. 16B shows a perspective view of the MCS device 22 and its catheter shaft 16 loaded within the insertion catheter 2832. FIGS. 16C and 16D show side views corresponding to FIGS. 16A and 16B. The side view of FIG. 16C in particular shows how the tubular body of the insertion catheter 2832 is adapted to receive the variable outer diameter of the MCS device 22 and its catheter shaft 16. Specifically, and with reference to FIGS. 15A-15C, FIG. 16C shows how the distal portion 2856 and distal transition section 2855 of the insertion catheter 2832 are configured to receive the enlarged diameter of the MCS device 22 while portions/sections proximal to the distal portion 2856 need only be sized large enough to fit the catheter shaft 16 therethrough. In such a configuration, the MCS device 22 and its catheter shaft 16 can be positioned within the insertion catheter 2832 by advancing the insertion catheter 2832 distally over the catheter shaft 16 until the MCS device 22 is contained in the distal portion 2856 and no further distal movement is achieved, for example, until the proximal end of the MCS device 22 physically abuts the inner surface of the distal transition section 2855. Such an interaction between the inner surface of the distal transition section 2855 and the proximal end of the MCS device due to the variable diameter of the tubular body 2836 (e.g., an enlarged distal diameter) can also facilitate loading of the insertion catheter 2832 with the MCS device 22 within an introducer sheath as described herein by allowing for longitudinal force applied to the insertion catheter 2832 to be transferred to the back of the MCS device 22.

In some embodiments of the circulatory support system, the MCS device can be packaged pre-positioned within the insertion catheter 2832 as shown in FIGS. 16B and 16D. When fully positioned within the insertion catheter 2832, the distal end of the MCS device 22 can extend past the distal end of the insertion catheter 2832 as shown. In some embodiments, a guidewire aid 38 can be pre-loaded within the MCS device and shaft 16 as described herein with the distal end 2857 of the insertion catheter 2832 received by proximal opening 90 of the guidewire aid 38.

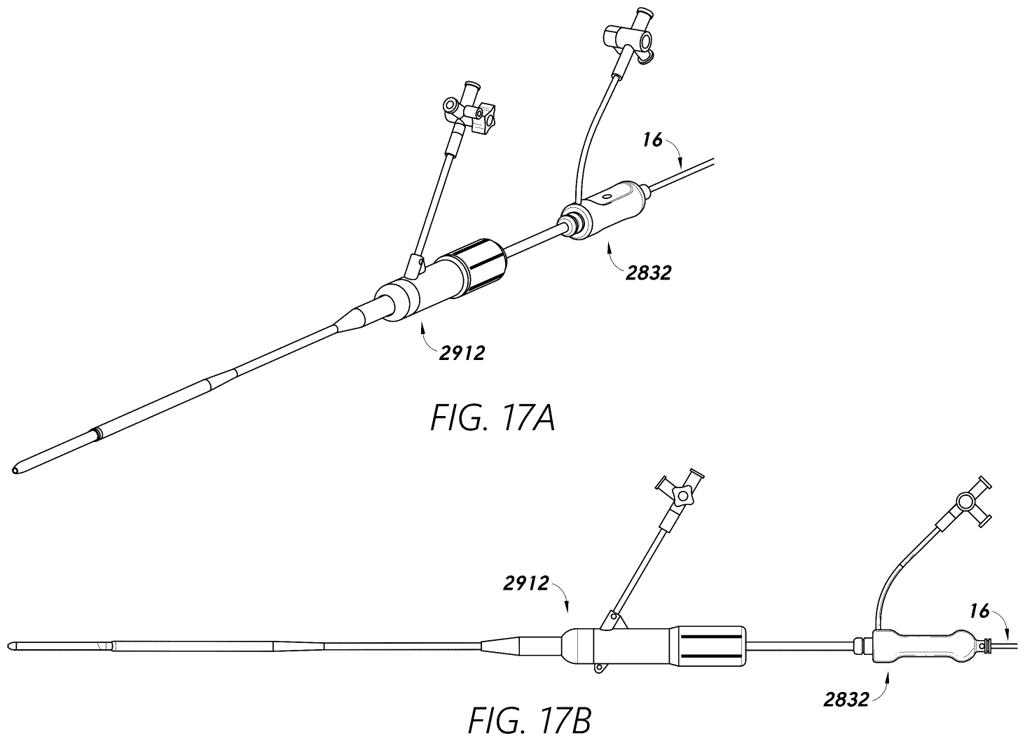

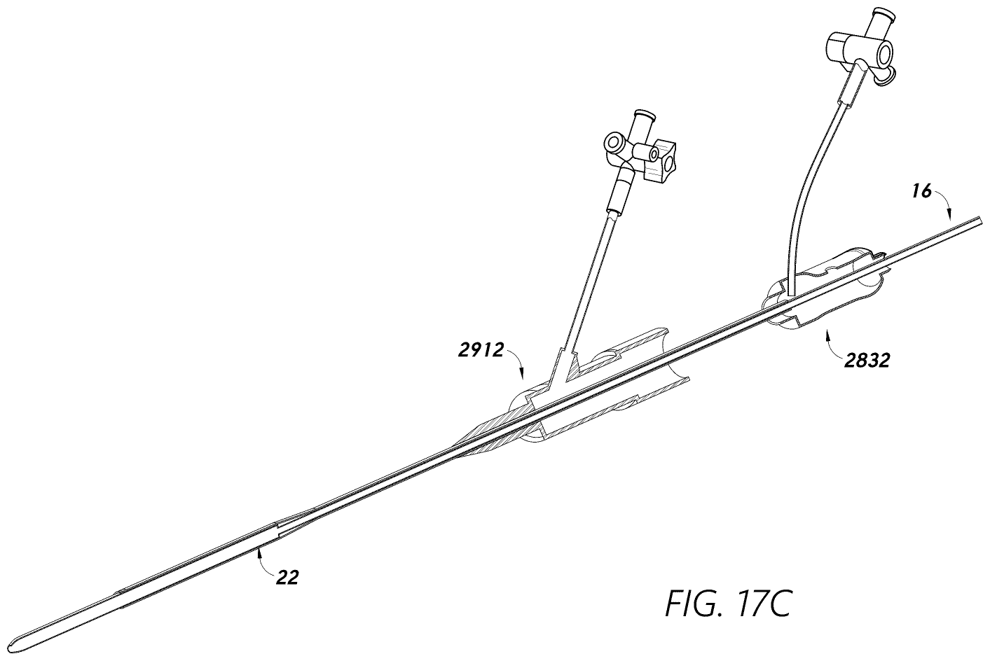

FIGS. 17A-17C are various views of an MCS device 22 and its catheter shaft 16 placed within the insertion catheter 2832 and partially within introducer sheath 2912 (which can be interchanged with any of the other introducer sheaths described herein). FIG. 17A shows a perspective view, FIG. 17B a side view, and FIG. 17C a perspective cross-sectional view of such an assembly. The introducer sheath 2912 shown is configured to be expandable, and as can be seen in FIGS. 17A-17C, the tubular body of the introducer sheath has expanded to fit the enlarged diameter of the MCS device 22 inside the insertion catheter 2832 and collapsed down upon the exterior of the insertion catheter 2832 where it has a smaller diameter. Also shown is a potential configuration of the first and second engagement structures as described herein of the insertion catheter 2832 and introducer sheath 2912, respectively, for locking the insertion catheter 2832 with (e.g., into) the introducer sheath 2912. As shown, the first engagement structure at the distal end of the hub of the insertion catheter 2832 can have a transvers cross-section that is configured to mate into the complementary second engagement structure at the proximal end of the hub of the introducer sheath 2912 and prevent rotation. For example, the transverse cross-section may have a non-circular shape such as an oval, triangle, square, hexagon, octagon, or a shape with ridges or other radial asymmetry that prevents rotation of the first engagement structure within the second engagement structure.

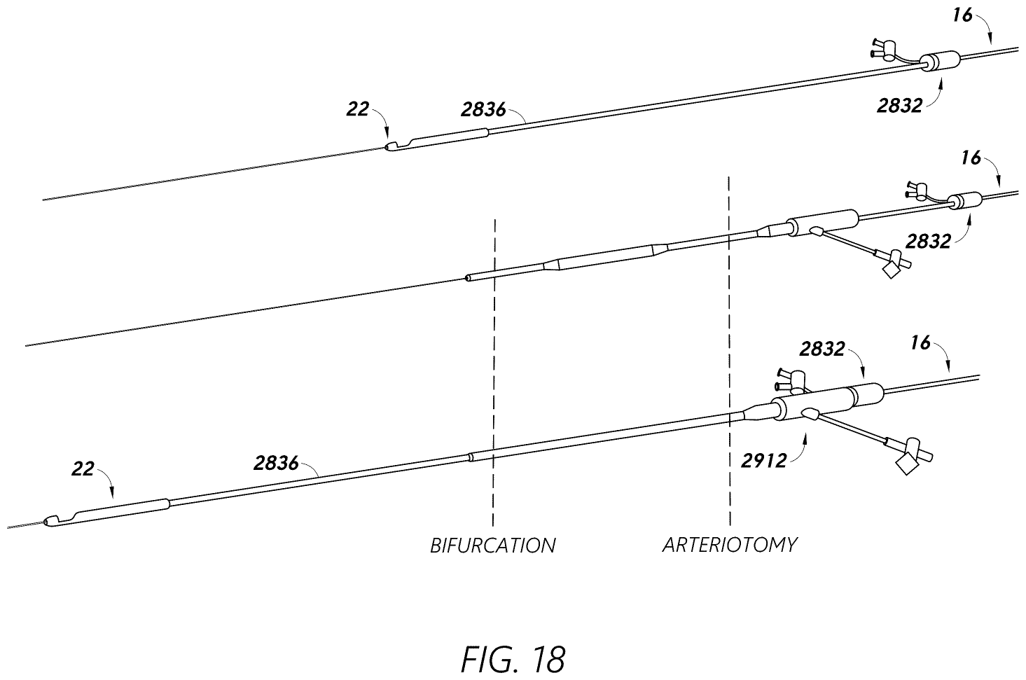

FIG. 18 illustrates an MCS device 22 and its catheter shaft 16 placed within the insertion catheter 2832 not inserted (at top), partially inserted (at middle), and fully inserted (at bottom) within an introducer sheath 2912 (which can be interchanged with any of the other introducer sheaths described herein). The partially inserted configuration shown at middle can correspond to the partially inserted configuration shown in FIGS. 17A-17C. As described herein, the MCS device 22 and its catheter shaft 16 can come pre-packaged within the insertion catheter 2832. The image at top shows this pre-packaged assembly after a guidewire has been loaded through the MCS device 22 and its catheter shaft 16. After an arteriotomy has been performed to gain access to a femoral artery and the distal end of the introducer sheath 2912 advanced through the arteriotomy, the distal end of the insertion catheter 2832 with MCS device 22 and catheter shaft 16 positioned inside can be inserted through the proximal end of the introducer sheath. The insertion catheter 2832 with MCS device 22 and catheter shaft 16 positioned inside can then be advanced distally through the introducer sheath 2912 and any hemostatic valves it may contain until the insertion catheter 2832 cannot advance further, such as by locking/docking into the introducer sheath 2912 as described herein (corresponding to the bottom image).

The insertion catheter 2832 can be configured such that the distal end of the tubular body 2836 of the insertion catheter 2832 extends past the distal end of the introducer sheath 2912 when the insertion catheter 2832 is fully docked with the introducer sheath 2912. Such a configuration can advantageously minimize the amount of push force required to advance the MCS device 22, as there is no need to push the MCS device individually through any portion of the lumen of the introducer sheath 2912. Instead, the MCS device is advanced entirely through the lumen of the introducer sheath 2912 while positioned within the insertion catheter 2832.

Also shown are features of the insertion catheter 2832 that can improve the safety and reliability of the procedure. For example, to keep the arteriotomy as small in diameter as possible, the insertion catheter 2832 can be configured such that its proximal portion 2852 (which can have an enlarged diameter relative to the mid portion 2854) remains outside the body when the insertion catheter 2832 is fully docked with the introducer sheath 2912. As another example, to minimizing blocking of interior lumens of arteries in which the devices pass through so as to reduce the risk of ischemia occurring (e.g., lower limb ischemia) during use of the system, the insertion catheter 2832 can be configured such that the mid portion 2854 (which can have a reduced diameter relative to the proximal and distal portions) extends from outside the body, through the arteriotomy of the femoral artery, through the femoral artery, through the bifurcation of the femoral artery where it joins the aorta, and into the aorta when the insertion catheter 2832 is fully docked with the introducer sheath 2912 (see dotted lines indicating “Arteriotomy” and “Bifurcation” in FIG. 18). Additionally, the insertion catheter 2832 can be configured such that the distal portion 2856 (which can have the largest diameter of the tubular body of the insertion catheter) is positioned within the aorta, which has a larger interior lumen than the femoral artery and can accommodate the size of the distal portion without risk of ischemia. From this position the MCS device 22 can be advanced distally out of the insertion catheter 2832 to its desired position within the body (e.g., across the aortic valve), and the insertion catheter 2832 can lock the catheter shaft 16 in place as described herein to prevent axial and/or rotational movement thereof, and the introducer sheath can lock the insertion catheter 2832 in place as described herein to prevent axial and/or rotational movement thereof, and the introducer sheath can be fixed, for example sutured, to the patient's body, thus fixing the catheter shaft 16 position with respect to the patient. When the procedure is complete, removal of the MCS device 22 and its catheter shaft 16 can be the reverse of insertion, where the MCS device 22 is first pulled proximally until it is seated within the insertion catheter 2832, after which the insertion catheter 2832 and MCS device 22 and its catheter shaft 16 can be removed from the body.

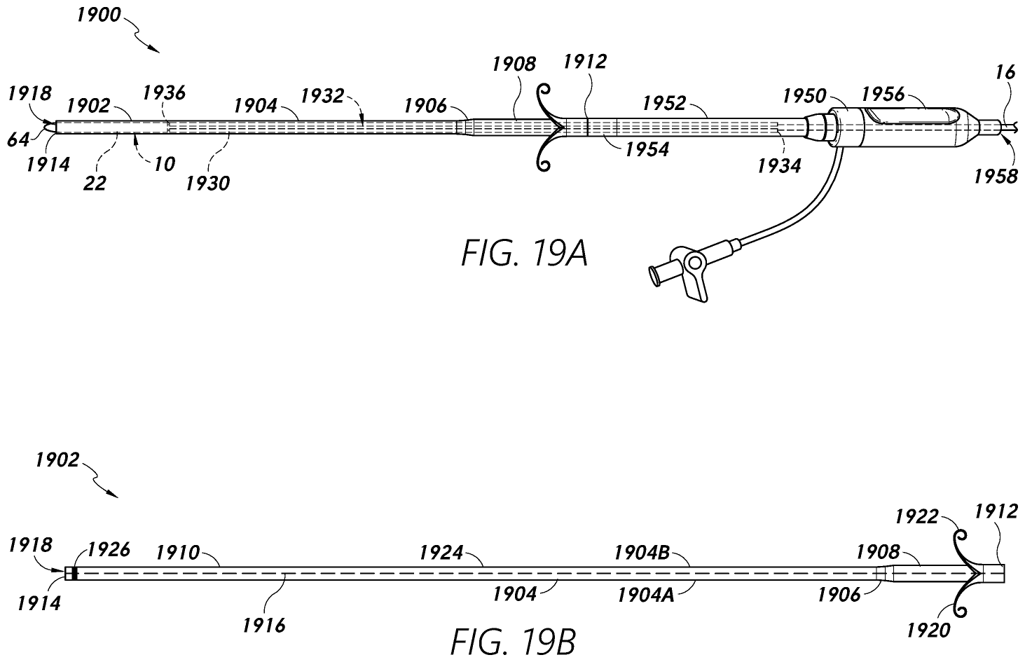

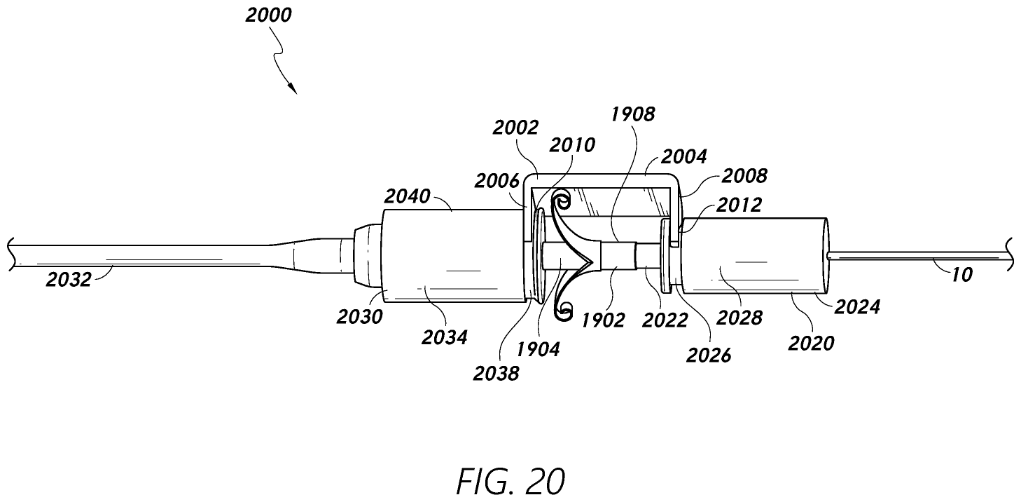

FIGS. 19A, 19B and 20 show use of a peel-away catheter configured to slidably receive at least a portion of a MCS system to facilitate delivery of the MCS system. The peel-away catheter can comprise a catheter shaft configured to be splitable and/or separable into multiple elongate shaft portions. In some instances, a medical delivery system can comprise an introducer sheath, an insertion tool, and a peel-away catheter comprising a catheter shaft having a portion disposed within the introducer sheath while a proximal portion of the catheter shaft is engaged with the insertion tool. The medical delivery system can comprise a spacer configured to axially space the insertion tool from the introducer sheath.

FIG. 19A is a side view of a delivery assembly 1900 comprising a peel-away catheter 1902 configured to facilitate delivery of an MCS system 10 to a target position. FIG. 19B is a side view of the peel-away catheter 1902. In some instances, the peel-away catheter 1902 can be configured to slidably receive at least a portion of the MCS system 10, such as at least a portion of a shaft 16 and pump 22 of the MCS system. The peel-away catheter 1902 can improve push-ability of the MCS system 10 and/or reduce or prevent damage to the MCS system 10, while the MCS system 10 is navigated through a tortuous vascular pathway. As described herein, at least a portion of the peel-away catheter 1902 can be received by an introducer sheath, including an expandable introducer sheath. The peel-away catheter 1902 can protect the MCS system 10 while at least a portion of the MCS system 10 is advanced through the introducer sheath. The peel-away catheter 1902 can be removed from around the MCS system 10, including from around the MCS device shaft 16 and/or pump 22, such as after deployment of the MCS system 10 through the introducer sheath.

Referring to FIG. 19A, the delivery assembly 1900 can comprise an insertion tool 1950 and the peel-away catheter 1902. The peel-away catheter 1902 can comprise a catheter shaft 1904 having a sheath delivery lumen 1918 extending through the catheter shaft 1904. A proximal portion 1906 of the catheter shaft 1904 can be configured to mate with the insertion tool 1950. In some instances, the proximal portion 1906 of the catheter shaft 1904 can be configured to mate with a portion of the insertion tool 1950 having a tubular configuration. For example, the insertion tool 1950 can comprise an insertion tool shaft 1952 and an insertion tool hub 1956, the insertion tool shaft 1952 extending distally from the insertion tool hub 1956. The proximal portion 1906 of the catheter shaft 1904 can be mated with a distal portion 1954 of the insertion tool shaft 1952. The insertion tool 1950 can comprise an insertion tool delivery lumen 1958 extending therethrough. For example, the proximal portion 1906 of the catheter shaft 1904 can be mated with a distal portion 1954 of the insertion tool shaft 1952 such that the sheath delivery lumen 1918 can be aligned with the insertion tool delivery lumen 1958. The MCS system 10 can be preloaded in the insertion tool 1950 and peel-away catheter 1902 for advancement to a target location.

In some instances, a support sleeve 1930 can be configured to be positioned around at least a portion of the shaft of the MCS system 10 to facilitate advancement of the MCS system 10 to the target location, providing column strength reinforcement and/or protect the MCS system 10. In some instances, the support sleeve 1930 can be disposed around a portion of the shaft 16 of the MCS system 10. The support sleeve 1930 can comprise a slit 1932 to it facilitate removal from around the MCS system 10. In some instances, a portion of the support sleeve 1930 can be received within the insertion tool delivery lumen 1958. Another portion of the support sleeve 1930 can be received in the sheath delivery lumen 1918. For example, a proximal end 1934 of the support sleeve 1930 can be in the insertion tool delivery lumen 1958. A distal end 1936 of the support sleeve 1930 can be configured to be received within the sheath delivery lumen 1918.

Referring to FIG. 19B, a side view of the peel-away catheter 1902 is shown. The catheter shaft 1904 can be removable/peelable/splitable to allow removal of the catheter shaft 1904 from around the MCS system 10. In some instances, the catheter shaft 1904 can be configured to split and/or separate along at least one longitudinal portion extending along an entire length of the catheter shaft 1904 to allow removal of the catheter shaft 1904 from around the MCS system 10. The longitudinal portion can extend along a dimension, including along a linear path, parallel or substantially parallel to a longitudinal axis of the catheter shaft 1904. The catheter shaft 1904 can be configured to split and/or separate along one or more longitudinal portions to allow removal of the catheter shaft 1904. In some instances, the catheter shaft 1904 can split and/or separate along one longitudinal portion such that the catheter shaft 1904 can be removed from around the MCS device by sliding the MCS system 10, such as the shaft 16 and/or pump 22 of the MCS system 10, through a slit formed by adjacent portions of the catheter shaft 1904 separating and/or splitting apart. Alternatively, the catheter shaft 1904 can split and/or separate along more than one longitudinal portion. For example, the catheter shaft 1904 can be split and/or separated into two or more pieces after the catheter shaft 1904 splits and/or separates along each of a plurality of longitudinal portions. The catheter shaft 1904 can be split and/or separated into various number of elongate shaft portions, including two, three or four pieces. In some instances, the catheter shaft 1904 can comprise two longitudinal portions 1916 along which the catheter shaft 1904 can split and/or separate. One longitudinal portion 1916 is shown in FIG. 19B. The other longitudinal portion 1916 can be on a portion of the catheter shaft 1904 not shown in FIG. 19B. For example, the two longitudinal portions 1916 can be on opposingly oriented portions of the catheter shaft 1904. Alternatively, the two longitudinal portions 1916 can be respective portions of the catheter shaft 1904 that are not opposingly oriented.

In some instances, a longitudinal portion can have the same or similar material as adjacent portions of the catheter shaft 1904. For example, the material of the catheter shaft 1904 can facilitate separation and/or splitting along the longitudinal portion. In some instances, the catheter shaft 1904 can comprise a material configured to propagate a tear from an initial proximal slit to allow separation of the catheter shaft 1904 into a plurality of elongate shaft portions. Alternatively, a longitudinal portion can comprise a weakened portion to facilitate separation and/or splitting of the catheter shaft 1904 along the longitudinal portion.

Referring again to FIG. 19B, the peel-away catheter 1902 can comprise a first and second operator engagement handle 1920, 1922. The first and second operator engagement handles 1920, 1922 can be coupled to respective portions of the catheter shaft 1904, such as the proximal portion 1906, and comprising at least a portion that extends laterally from the catheter shaft 1904. The first operator engagement handle 1920 can be coupled to a first elongate shaft portion 1904A. The second operator engagement handle 1922 can be coupled to a second elongate shaft portion 1904B. An operator can engage with the first and second operator engagement handles 1920, 1922 to pull apart the catheter shaft 1904 along each longitudinal portion 1916. For example, the operator can pull the first and second operator engagement handles 1920, 1922 away from one another to split and/or pull apart the catheter shaft 1904 into the first and second elongate shaft portion 1904A, 1904B, respectively.

As described herein, the proximal portion 1906 of the catheter shaft 1904 can be mated with a distal portion 1954 of the insertion tool shaft 1952. In some instances, the proximal portion 1906 can have an insertion tool mating portion 1908 configured to mate with the distal portion 1954 of the insertion tool shaft 1952. The insertion tool mating portion 1908 can have a larger lateral cross-sectional size (e.g., cross-section perpendicular to the longitudinal axis of the catheter shaft 1904) than more distal portions of the catheter shaft 1904. For example, an outer diameter and/or inner diameter of the insertion tool mating portion 1908 can be larger than that of more distal portions of the catheter shaft 1904. The insertion tool mating portion 1908 can provide an interference fit with the distal portion 1954 of the insertion tool shaft 1952 to provide hemostatic coupling between the two. For example, a diameter of the portion of the delivery lumen 1918 extending through the insertion tool mating portion 1908 can be sized to be disposed over and have the interference fit with an exterior surface of the distal portion 1954 of the insertion tool shaft 1952 and thereby provide the hemostatic coupling. In some instances, the insertion tool mating portion 1908 can have an inner and/or outer lateral cross-sectional size which increases in size along a longitudinal direction extending to a proximal end 1912 of the catheter shaft 1904, such as to provide a flared configuration. The flared configuration can facilitate providing the interference fit and/or hemostatic coupling with the insertion tool shaft 1952.

In some alternative instances, a proximal portion of a catheter shaft can be configured to be inserted into a distal portion of an insertion tool shaft to provide an interference fit, for example a sidewall of the distal portion of the insertion tool shaft being over an exterior surface of the proximal portion of the catheter shaft. In some alternative instances, an interference fit between a proximal portion of a catheter shaft and a distal portion of an insertion tool shaft can comprise a butt joint that is peelable, such as radially peelable, such the proximal portion of the catheter shaft can be removed from the distal portion of the insertion tool shaft.

A length and/or cross-sectional size of the catheter shaft 1904 can be selected based at least in part on a size of the MCS system 10 slidably received within the delivery lumen 1918. The length and/or inner diameter of the catheter shaft 1904 (e.g., cross-sectional size of the delivery lumen 1918) can be configured to accommodate the MCS system 10 and/or the support sleeve 1930. In some instances, a distal tip 64 of the MCS system 10 can be aligned with the distal end 1914 of the catheter shaft 1904. For example, at least a portion of the distal tip 64, such as at least a portion of a nose cone distal portion of the MCS system 10, can be disposed distally of the catheter shaft 1904 while the MCS system 10 is preloaded in the catheter shaft 1904. In some instances, a proximal end of the distal tip 64 can be aligned with the distal end 1914 of the catheter shaft 1904, such that the distal tip 64 is distal of the catheter shaft 1904. For example, the nose cone distal portion of the MCS system 10 can be disposed distally of the catheter shaft 1904 while the MCS system 10 is preloaded in the catheter shaft 1904.

In some instances, at least a portion of an exterior surface 1924 of the catheter shaft 1904 can comprise a hydrophilic coating. In some instances, the hydrophilic coating can facilitate navigation of the catheter shaft 1904, including through an introducer sheath. In some instances, the catheter shaft 1904 can comprise one or more markers 1926, including radiopaque markers, to facilitate visualizing a position of the catheter shaft 1904. In some instances, the markers 1926 can be on and/or coupled to the exterior surface 1924 of the catheter shaft 1904. For example, the catheter shaft 1904 can comprise a plurality of radiopaque markers arranged at respective positions along a longitudinal dimension of the catheter shaft 1904 to facilitate visualizing advancement of the catheter shaft 1904 through an introducer sheath. In some instances, one or more markers 1926 can be on the distal portion 1910, including at or proximate to the distal end 1914, of the catheter shaft 1904.

FIG. 20 is a side view of a delivery system 2000 having an MCS system 10 preloaded therethrough. The delivery system 2000 can comprise a spacer 2002, the peel-away catheter 1902 describe with reference to FIGS. 19A and 19B, an insertion tool 2020, and an introducer sheath 2030. The peel-away catheter 1902 can be mated with the insertion tool 2020. The insertion tool 2020 can comprise an insertion tool shaft 2022 extending distally from an insertion tool hub 2024. For example, the insertion tool mating portion 1908 of the catheter shaft 1904 can be mated with the insertion tool shaft 2022. Mating between the peel-away catheter 1902 and the insertion tool shaft 2022 can be the same as or similar to that between the peel-away catheter 1902 and the insertion tool shaft 1952 described with reference to FIGS. 19A and 19B. A portion of the catheter shaft 1904 of the peel-away catheter 1902, including the distal portion 1910, can be disposed through a delivery lumen 2036 (not shown) of the introducer sheath 2030. The introducer sheath 2030 can comprise an introducer sheath shaft 2032 extending distally from an introducer sheath hub 2034. A portion of the catheter shaft 1904 can be disposed through at least a portion of the delivery lumen 2036 extending through the introducer sheath shaft 2032 and the introducer sheath hub 2034.

The spacer 2002 can comprise at least a portion configured to be positioned between the introducer sheath hub 2034 and the insertion tool hub 2024 to axially space the introducer sheath hub 2034 from the insertion tool hub 2024 while the insertion tool mating portion 1908 of the catheter shaft 1904 is mated with the insertion tool shaft 2022. The spacer 2002 can be configured to maintain a fixed distance between the introducer sheath hub 2034 and the insertion tool hub 2024, for example such that the introducer sheath hub 2034 is distal of and aligned with the insertion tool hub 2024 along a longitudinal axis of the introducer sheath hub 2034 and/or the insertion tool hub 2024. Spacing and/or alignment can be maintained during navigation of the introducer sheath 2030 carrying the catheter shaft 1904 and MCS system 10. The spacer 2002 can be removed after the introducer sheath 2030 is advanced to the target position, such as to allow advancement of the catheter shaft 1904, insertion tool 2020 and/or MCS system 10. In some alternative instances, after the introducer sheath 2030 is navigated to a target position, such as while carrying a dilator extending therethrough, the peel-away catheter 1902 coupled to the insertion tool 2020 can be advanced into the introducer sheath 2030. For example, the dilator can be withdrawn and the peel-away catheter 1902 having the MCS system 10 preloaded therethrough can be advanced at least partially through the introducer sheath 2030. The spacer 2002 can be engaged with the introducer sheath hub 2034 and the insertion tool hub 2024 to maintain an axial spacing and/or alignment between the two. The spacer 2002 can be removed to facilitate peeling away and/or removal of the peel-away catheter 1902, such as after the MCS system 10 is deployed from the peel-away catheter 1902.

The spacer 2002 can comprise a distal portion 2006 configured to engage with the introducer sheath hub 2034 and a proximal portion 2008 configured to engage with the insertion tool hub 2024. A medial portion 2004 can extend between and coupling the proximal portion 2008 and distal portion 2006. The distal portion 2006 can comprise a first hub engagement feature 2010 configured to engage the introducer sheath hub 2034, including a corresponding spacer engagement feature 2038 of the introducer sheath hub 2034. The proximal portion 2008 can comprise a second hub engagement feature 2012 configured to engage with the insertion tool hub 2024, including a corresponding spacer engagement feature 2026 of the insertion tool hub 2024. In some instances, the spacer engagement feature 2038 can be on a proximal portion 2040 of the introducer sheath hub 2034. In some instances, the spacer engagement feature 2026 can be on a distal portion 2028 of the insertion tool hub 2024.

In some instances, the spacer engagement feature 2038, 2026 of each of the introducer sheath hub 2034 and the insertion tool hub 2024 can comprise a groove and/or recess extending at least partially circumferentially around, including around an entire circumference of, a portion of the respective hub. The first and second hub engagement features 2010, 2012 can comprise at least a portion configured to be received by and/or within the respective groove and/or recess to mate and/or couple with the groove and/or recess. In some instances, at least a portion of the distal portion 2006 and proximal portion 2008 can be oriented at an angle relative to, including being perpendicular or substantially perpendicular to, the medial portion 2004. In some instances, while the spacer 2002 is engaged with the insertion tool hub 2024 and the introducer sheath hub 2034, at least a portion of the medial portion 2004 can be oriented along a direction parallel or substantially parallel to the longitudinal axis of the insertion tool hub 2024 and/or the introducer sheath hub 2034. At least a portion of each of the distal portion 2006 and the proximal portion 2008 can be received within a respective groove and/or recess of the introducer sheath hub 2034 or insertion tool hub 2024. The first hub engagement feature 2010 can comprise the edge of the distal portion 2006 received within the groove and/or recess of the introducer sheath hub 2034. The second hub engagement feature 2012 can comprise the edge of the proximal portion 2008 received in the groove and/or recess of the insertion tool hub 2024. The edges of the distal portion 2006 and proximal portion 2008 can be configured to have complement shape and/or size of the respective groove and/or recess. For example, the edge of the distal portion 2006 and/or proximal portion 2008 received within the groove and/or recess of the introducer sheath hub 2034 and/or insertion tool hub 2024 can have a concave curvature configured to mate with a groove and/or recess extending along a path having a convex shape. Alternatively or in combination, the spacer engagement features can have any number of other different configurations, including slots, indentations, etc.

It will be understood that peel-away catheters described herein can be used in combination with one or more other insertion tools and/or introducer sheaths described herein.

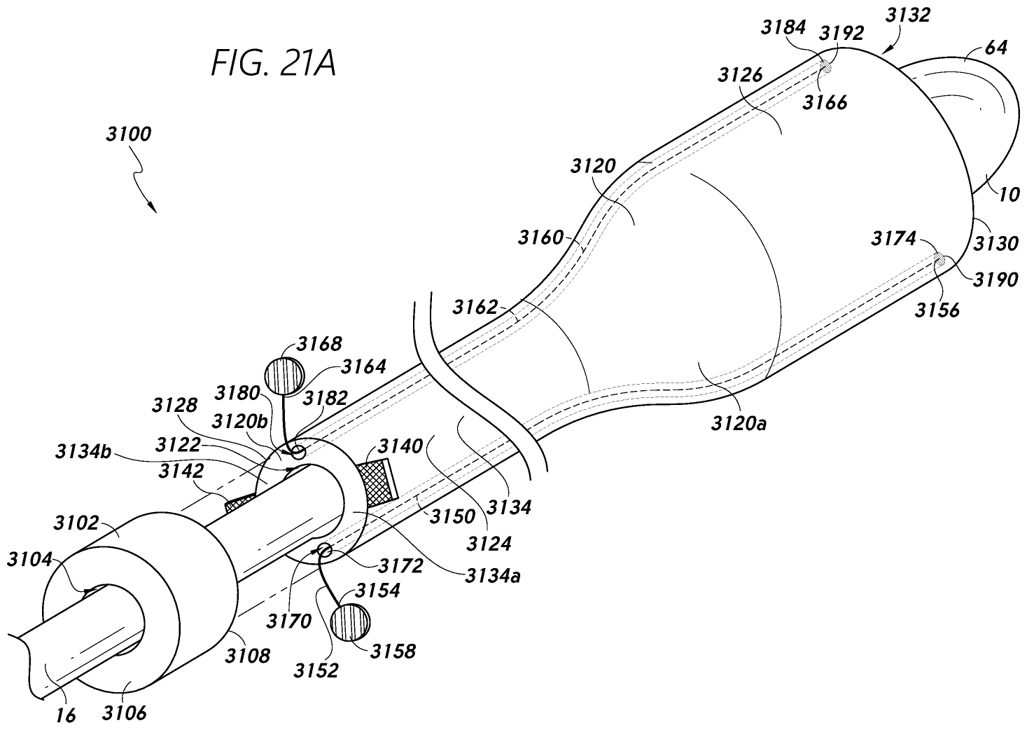

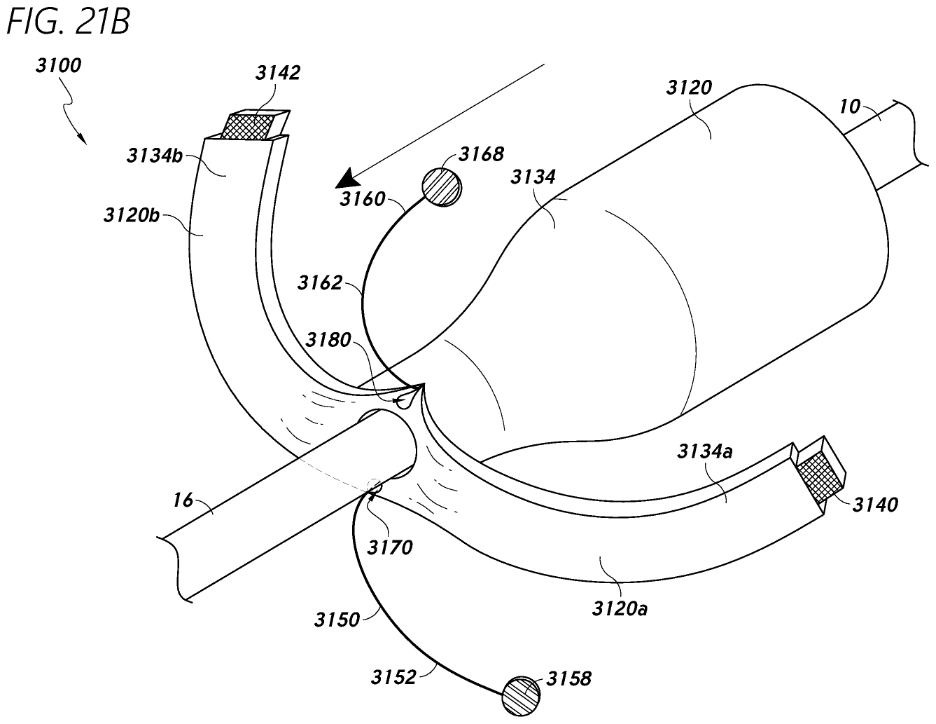

FIGS. 21A to 28 illustrate various examples of peel-away catheters (e.g., shaft, jacket, or sheath) that can be used for delivering an MCS system described herein independently of an insertion tool.

FIG. 21A is an exploded perspective view of an example of a peel-away catheter 3100 and an MCS system 10 preloaded through the peel-away catheter 3100. FIG. 21B is a perspective view of a catheter shaft 3120 of the peel-away catheter 3100 being peeled and/or removed from around the MCS system 10, including from around the shaft 16 of the MCS system 10. The peel-away catheter 3100 can comprise a first elongate member 3150 and a second elongate member 3160 each comprising a portion configured to be received within a respective elongate member lumen 3170, 3180 of the catheter shaft 3120. As described in further detail herein, an operator can pull on the elongate members 3150, 3160 to facilitate splitting and/or separating the catheter shaft 3120 into a plurality of elongate shaft portions. FIG. 21A shows the catheter shaft 3120 in an unpeeled configuration. FIG. 21B shows use of the first and second elongate members 3150, 3160 to split and/or separate the catheter shaft 3120 into a first and second elongate shaft portion 3120a, 3120b, so as to facilitate peeling and/or removal of the catheter shaft 3120 from around the MCS system 10.

The first elongate member 3150 can comprise a first elongate portion 3152. A first proximal elongate member tab 3158 can be coupled to a proximal portion of the first elongate portion 3152, including to a proximal end 3154, of the first elongate portion 3152. The second elongate member 3160 can comprise a second elongate portion 3162. A second proximal elongate member tab 3168 can be coupled to a proximal portion of the second elongate portion 3162, including to a proximal end 3164, of the second elongate portion 3162. The catheter shaft 3120 can comprise a first elongate member lumen 3170 extending along a first longitudinal portion of a shaft wall 3134 of the catheter shaft 3120. The catheter shaft 3120 can comprise a second elongate member lumen 3180 extending along a second longitudinal portion of the shaft wall 3134. For example, corresponding portions of the shaft wall 3134 can define the first and second elongate member lumens 3170, 3180. At least a portion of the first elongate portion 3152 can be configured to be received within the first elongate member lumen 3170. At least a portion of the second elongate portion 3162 can be configured to be received within the second elongate member lumen 3180. The first and second proximal elongate member tabs 3158, 3168 can be disposed externally of the catheter shaft 3120 to facilitate manipulation (e.g., grabbing the first and second proximal elongate member tabs 3158, 3168 and pulling the first and second elongate members 3150, 3160) by an operator. For example, the first and second proximal elongate member tabs 3158, 3168 and/or a portion of the first and/or second elongate portions 3152, 3162 can be disposed externally of the catheter shaft 3120 (as shown in FIG. 21A), including proximally of the sheaths shaft 3120. In some instances, a portion of each of the first and/or second elongate portions 3152, 3162 can be disposed through a respective opening at proximal ends 3172, 3182 of the first and second elongate member lumens 3170, 3180, such that the first and second proximal elongate member tabs 3158, 3168 are disposed externally of the catheter shaft 3120. For example, proximal ends 3172, 3182 of the first and second elongate member lumens 3170, 3180 can be at a proximal end 3128 of the catheter shaft 3120.

A distal end 3156 of the first elongate portion 3152 can be coupled to the catheter shaft 3120 and a distal end 3166 of the second elongate portion 3162 can be coupled the catheter shaft 3120 such that the first and second elongate portions 3152, 3162 can be tensioned and pulled laterally away from a longitudinal axis of the catheter shaft 3120 for splitting and/or separating the catheter shaft 3120 into a first elongate shaft portion 3120a and a second elongate shaft portion 3120b as shown in, for example, FIG. 21B. For example, the shaft wall 3134 can be split and/or separated into a first elongate shaft wall portion 3134a and a second elongate shaft wall portion 3134b. In some instances, the distal end 3156 of the first elongate portion 3152 can be coupled to the catheter shaft 3120 at a first location on a distal portion 3126 of the catheter shaft 3120 including a first location of a distal portion of the shaft wall 3134. For example, the distal end 3156 of the first elongate portion 3152 can be coupled to a first location of a distal portion of the shaft wall 3134 defining a distal end 3174 of the first elongate member lumen 3170. In some instances, the distal end 3166 of the second elongate portion 3162 can be coupled to the catheter shaft 3120 at a second location on the distal portion 3126 of the catheter shaft 3120, including a second location of the distal portion of the shaft wall 3134. In some instances, the distal end 3166 of the second elongate portion 3162 can be coupled to a second location of the distal portion of the shaft wall 3134 defining a distal end 3184 of the second elongate member lumen 3180. The distal ends 3174, 3184 of the first and second elongate member lumens 3170, 3180, respectively, can be at or proximal of a distal end 3130 of the catheter shaft 3120. For example, the distal ends 3174, 3184 of the first and second elongate member lumens 3170, 3180, respectively, can be at or proximal of a distal end of the shaft wall 3134.

In some instances, the first and/or second elongate portions 3152, 3162 can be coupled to or include a respective marker 3190, 3192, including a radiopaque marker. In some instances, the markers 3190, 3192 can each comprise at least a portion disposed at or near the distal end 3174, 3184 of the respective elongate member lumen 3170, 3180. For example, the distal ends 3156, 3166 of the elongate portions 3152, 3162, respectively, can be coupled to the markers 3190, 3192 and/or a portion of the shaft wall 3134.

The peel-away catheter 3100 can comprise a first proximal shaft tab 3140 coupled to a first proximal portion of the catheter shaft 3120. Additionally or optionally, the peel-away catheter 3100 can comprise a second proximal shaft tab 3142 coupled to a second proximal portion of the catheter shaft 3120. For example, the first proximal shaft tab 3140 can be coupled to a proximal portion of the first elongate shaft wall portion 3134a. The second proximal shaft tab 3142 can coupled to a proximal portion of the second elongate shaft wall portion 3134b. The first and second proximal shaft tabs 3140, 3142 can be configured to be pulled laterally away from the longitudinal axis of the catheter shaft 3120 for separating the catheter shaft 3120 into first and second elongate shaft portions 3120a, 3120b, such as first and second elongate shaft wall portions 3134a, 3134b.

The first and second proximal shaft tabs 3140, 3142, and the first and second elongate members 3150, 3160 can be disposed at respective positions around a circumference of the catheter shaft 3120. In some instances, as shown, for example, in FIG. 21A, the first and second proximal shaft tabs 3140, 3142 can be disposed at opposing positions around the circumference of the catheter shaft 3120. In some instances, the first and second elongate members 3150, 3160 can be disposed at opposing positions around the circumference of the catheter shaft 3120.

The catheter shaft 3120 can extend distally from a catheter hub 3102. The catheter hub 3102 can comprise a hub delivery lumen 3104 extending therethrough, such as from a proximal end 3106 to a distal end 3108. A portion of the MCS system 10, such as a shaft 16 of the MCS system 10 can be slidably disposed through the hub delivery lumen 3104. The catheter hub 3102 may or may not be a peel-away hub.

In some instances, the catheter hub 3102 is not a peel-away hub. For example, while in the unpeeled configuration, the catheter shaft 3120 can extend distally from the catheter hub 3102. The catheter hub 3102 can be coupled to and/or engaged with the catheter shaft 3120, for example the distal end 3108 of the catheter hub 3102 can be coupled to and/or engaged with the proximal end 3128 of the catheter shaft 3120. The distal end 3108 of the catheter hub 3102 and/or the proximal end 3128 of the catheter shaft 3120 can comprise any number of engagement features configured to allow coupling between the catheter hub 3102 and catheter shaft 3120. In some instances, after the peel-away catheter 3100 carrying the MCS system 10 is advanced to a target location, the catheter hub 3102 can be separated, disengaged from and/or decoupled from the catheter shaft 3120 such that the catheter shaft 3120 can be remove from around the MCS system 10.

Referring to FIG. 21B, an operator can engage with the first and second proximal elongate member tabs 3158, 3168 to pull the first and second elongate members 3150, 3160, including the first and second elongate portions 3152, 3162, laterally away from the longitudinal axis of the catheter shaft 3120 such that the first and second elongate portions 3152, 3162 can cut through corresponding portions of the shaft wall 3134 between the respective elongate member lumen 3170, 3180 and an exterior surface of the shaft wall 3134. The operator can engage with the first and second proximal shaft tabs 3140, 3142, such as to pull the first and second proximal shaft tabs 3140, 3142 laterally away from the longitudinal axis of the catheter shaft 3120 to cause portions of shaft wall 3134 between the respective elongate member lumen 3170, 3180 and an interior surface of the shaft wall 3134 to tear. In some instances, the interior surface of the shaft wall 3134 can comprise at least a portion that defines a shaft delivery lumen 3122 extending through the catheter shaft 3120. Corresponding portions of the MCS system 10 can be slidably disposed through the shaft delivery lumen 3122.

Cutting through corresponding portions of the shaft wall 3134 between the respective elongate member lumen 3170, 3180 and an exterior surface of the shaft wall 3134 and tearing corresponding portions of the shaft wall 3134 between the first and second elongate member lumens 3170, 3180 and an interior surface of the shaft wall 3134 can allow or facilitate separation of the shaft wall 3134 into the first and second elongate shaft wall portions 3134a, 3134b. In some instances, a thickness of portions of the shaft wall 3134 (for example, the distance between the outer surface of the shaft 3120 and the shaft delivery lumen 3122) along, or defining, the first and second elongate member lumens 3170, 3180 can be less than that of other portions the shaft wall 3134, to facilitate cutting through and/or tearing of the shaft wall 3134. For example, the thickness of the shaft wall 3134 of the longitudinal portions along which the first and second elongate member lumen 3170, 3180 extend can be thinner than that of other or adjacent wall portions, including that at a perpendicular position around the circumference of the catheter shaft 3120.

In some instances, splitting the catheter shaft 3120 into separate elongate shaft portions 3120a, 3120b can be performed incrementally. For example, a first longitudinal portion of the catheter shaft 3120 can be peeled and/or split. After the first longitudinal portion is split, remaining unpeeled portions of the catheter shaft 3120 can be translated proximally along the MCS system 10 to allow an additional longitudinal portion of the catheter shaft 3120 to be split. Additional longitudinal portions can be split until the entire catheter shaft 3120 is removed.

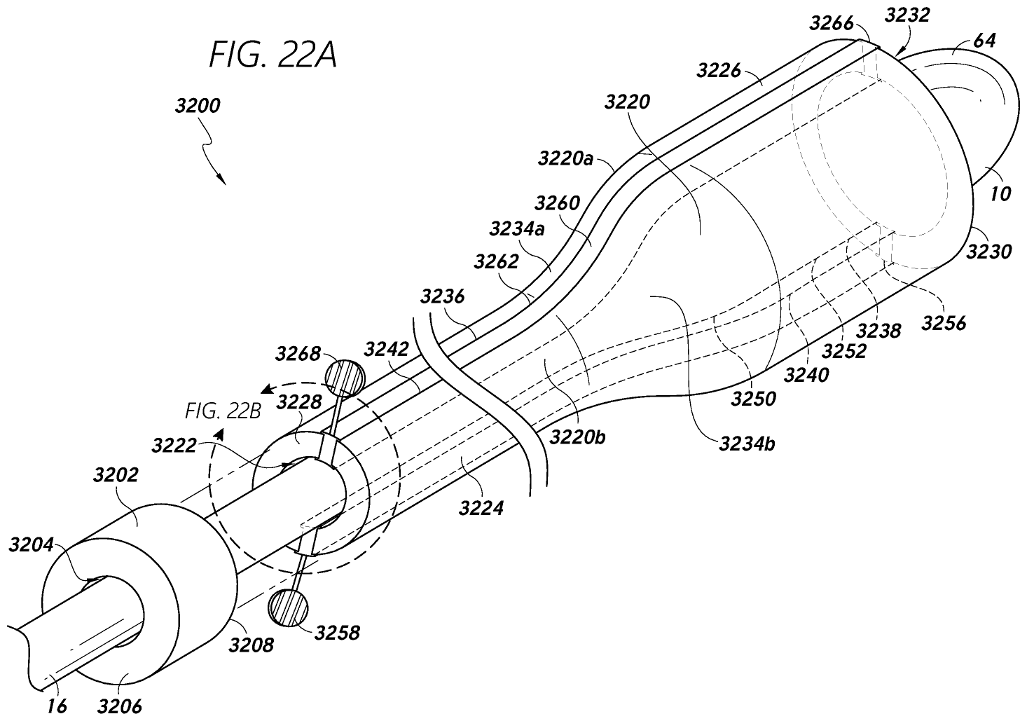

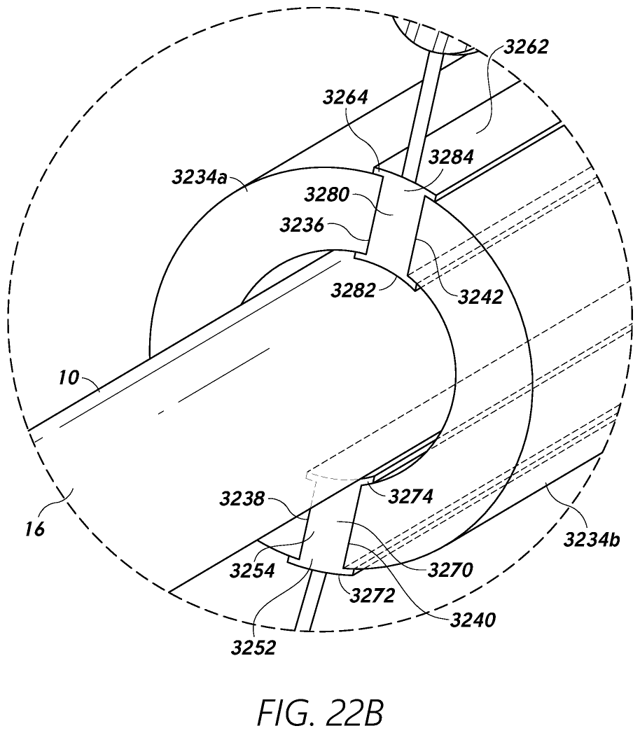

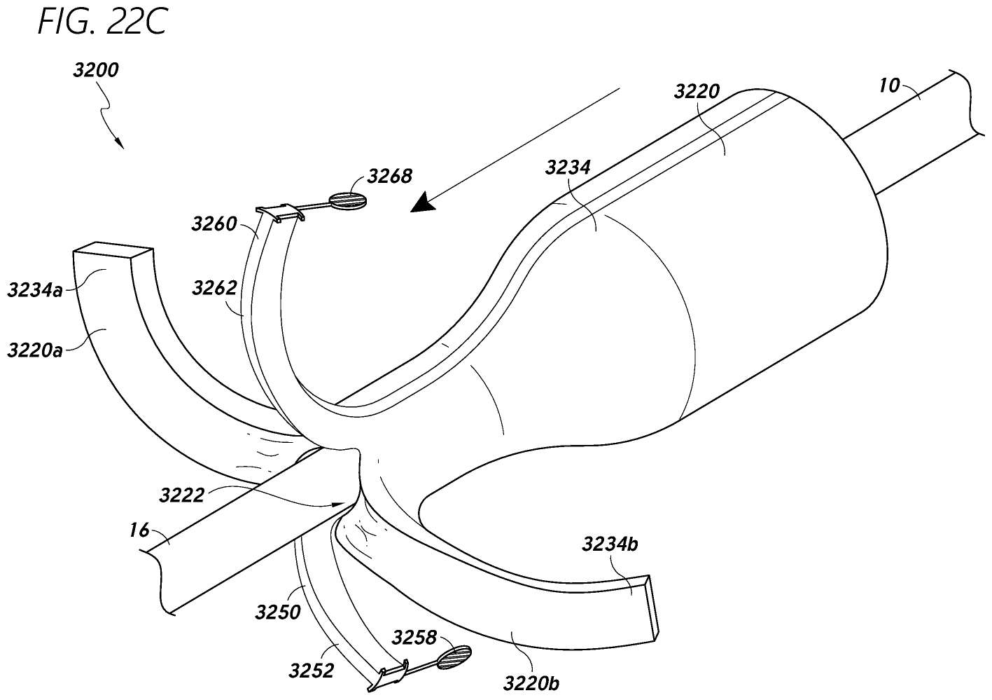

FIG. 22A is an exploded perspective view of an example of a peel-away catheter 3200 and an MCS system 10 preloaded through the peel-away catheter 3200. FIG. 22B is a more detailed view of a proximal portion of the catheter shaft. FIG. 22C is a perspective view of a catheter shaft 3220 of the peel-away catheter 3200 being peeled and/or removed from around the MCS system 10, including from around the shaft 16 of the MCS system 10. The peel-away catheter 3200 can comprise a first elongate member 3250 and a second elongate member 3260 each extending along a longitudinal portion of the catheter shaft 3220. As described in further detail herein, an operator can pull on the elongate members 3250, 3260 to facilitate splitting and/or separating the catheter shaft 3220 into a plurality of elongate shaft portions. FIGS. 22A and 22B show the catheter shaft 3220 in an unpeeled configuration. FIG. 22C shows separation of the first and second elongate members 3250, 3260 from remaining portions of the catheter shaft 3220 to split and/or separate the catheter shaft 3220, for example forming a first and second elongate shaft portion 3220a, 3220b separated from the first and second elongate members 3250, 3260.

The first elongate member 3250 can comprise a first elongate portion 3252. A first proximal elongate member tab 3258 can be coupled to a proximal portion of the first elongate portion 3252, including to a proximal end 3254, of the first elongate portion 3252. The second elongate member 3260 can comprise a second elongate portion 3262. A second proximal elongate member tab 3268 can be coupled to a proximal portion of the second elongate portion 3262, including to a proximal end 3264, of the second elongate portion 3262. At least a portion of each of the first and second elongate portions 3252, 3262 can extend along an entire or substantially entire longitudinal dimension, such as length, of the catheter shaft 3220. In some instances, each of the first and second elongate portions 3252, 3262 can form a wall portion of the catheter shaft 3220. For example, the catheter shaft 3220 can comprise a first elongate shaft wall portion 3234a and a second elongate shaft wall portion 3234b. The first and second elongate portions 3252, 3262 can be positioned between respective edges of the first and second elongate shaft wall portions 3234a, 3234b. For example, the second elongate portion 3262 can be positioned between, for example being sandwiched by, a first edge 3236 of the first elongate shaft wall portion 3234a and a second edge 3242 of the second elongate shaft wall portion 3234b. The first elongate portion 3252 can be positioned between, for example being sandwiched by, a second edge 3238 of the first elongate shaft wall portion 3234a and a first edge 3240 of the second elongate shaft wall portion 3234b. In some instances, the first and second elongate portions 3252, 3262 can be at opposing positions around a circumference of the catheter shaft 3220. In some instances, the first and second elongate portions 3252, 3262 and the first and second elongate shaft wall portions 3234a, 3234b can together form a sidewall of the catheter shaft 3220. In some instances, interior surfaces of the first and second elongate portions 3252, 3262, and interior surfaces of the first and second elongate shaft wall portions 3234a, 3234b can together define a shaft delivery lumen 3222 configured to slidably receive the MCS system 10. The first and second elongate portions 3252, 3262 can extend from a proximal end 3228 to a distal end 3230 of the catheter shaft 3220, including from a proximal end to a distal end of the elongate shaft wall portions 3434a, 3434b.

FIG. 22B shows the proximal portion 3224 of the catheter shaft 3220 in further detail. In some instances, a lateral cross-section of the first and/or second elongate portions 3252, 3262 can have end portions with widths that are larger than that of a medial portion extending between the end portions. The lateral cross-section can be taken along a plane perpendicular or substantially perpendicular to a longitudinal axis of the catheter shaft 3220. For example, the lateral cross-section of the first elongate portion 3252 can comprise a shape having a first end portion 3272, a second end portion 3274, and a medial portion 3270 between the first and second end portions 3272, 3274. The first end portion 3272 can be disposed further away from a longitudinal axis of the catheter shaft 3220 and the second end portion 3274 can be disposed closer to the longitudinal axis. In some instances, the first and second end portions 3272, 3274 can be opposingly oriented. A width, such as a dimension extending along the plane of the cross-section, of the first and second end portions 3272, 3274 can be larger than that of the medial portion 3270. In some instances, the lateral cross-section of the second elongate portion 3262 can comprise a first end portion 3282, a second end portion 3284, and a medial portion 3280 between the first and second end portions 3282, 3284. The first end portion 3282 can be disposed closer to the longitudinal axis of the catheter shaft 3220 and the second end portion 3284 can be disposed further away from the longitudinal axis. In some instances, the first and second end portions 3282, 3284 can be opposingly oriented. A width of the first and second end portions 3282, 3284 can be larger than that of the medial portion 3280. In some instances, portions of the first and second end portions 3272, 3274, 3282, 3284 of the lateral cross-sections of the first and second elongate portions 3252, 3262 can be disposed over and/or be in contact with portions of respective interior and exterior surfaces of the adjacent elongate shaft wall portions 3434a, 3434b, for example to facilitate securing the elongate portions 3252, 3262 to the first and second elongate shaft wall portions 3234a, 3234b. In some instances, the lateral cross-section of the first and/or second elongate portions 3252, 3262 can assume the shape of an “I-beam.”

In some instances, one or both of the first and second elongate portions 3252, 3262 can each comprise one or more markers, including radiopaque markers, to facilitate visualizing a position of the catheter shaft 3220. For example, one or both of the first and second elongate portions 3252, 3262 can each comprise one or more radiopaque markers coupled to and/or embedded within a portion thereof, including a distal portion, such as at a distal end 3256, 3266.

Referring to FIG. 22C, an operator can engage with the first and second proximal elongate member tabs 3258, 3268 to pull the first and second elongate members 3250, 3260, such as the first and second elongate portions 3252, 3262, laterally away from the longitudinal axis of the catheter shaft 3220. Pulling the first and second elongate portions 3252, 3262 laterally can separate the first and second elongate portions 3252, 3262 from the first and second elongate shaft wall portions 3234a, 3234b, to facilitate removal of the catheter shaft 3220 from around the MCS system 10. For example, the first elongate portion 3252 can be separated from the first edge 3236 of the first elongate shaft wall portion 3234a and the second edge 3242 of the second elongate shaft wall portion 3234b. The second elongate portion 3262 can be separated from the second edge 3238 of the first elongate shaft wall portion 3234a and the first edge 3240 of the second elongate shaft wall portion 3234b.

In some instances, removing the catheter shaft 3220 from around the MCS system 10 can be performed incrementally. For example, a first longitudinal portion of the catheter shaft 3220 can be peeled and/or split. After the first longitudinal portion is split, remaining unpeeled portions of the catheter shaft 3220 can be translated proximally along the MCS system 10 to allow an additional longitudinal portion of the catheter shaft 3220 to be split. Additional longitudinal portions can be split until the entire catheter shaft 3220 is removed.

It will be understood that although FIGS. 21A through 22C describe separating the catheter shafts 3120, 3220 into two elongate shaft portions, the catheter shafts 3120, 3220 can be separated into more elongate shaft portions. For example, peel-away catheters can comprise more elongate members configured to allow the catheter shafts to separate into more elongate shaft portions.

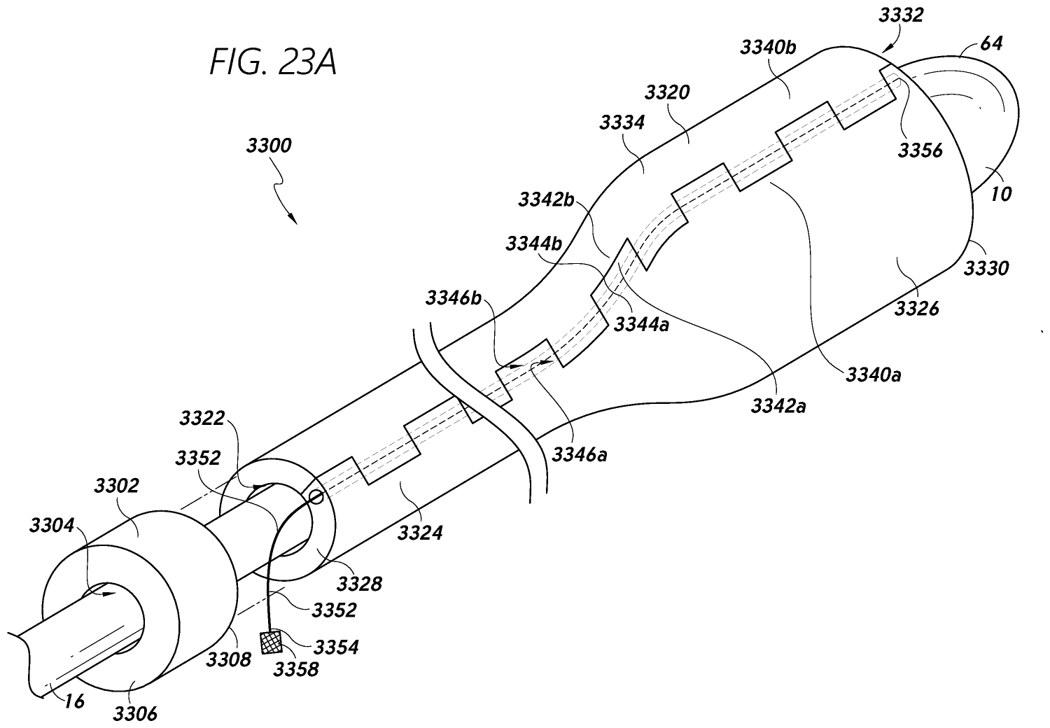

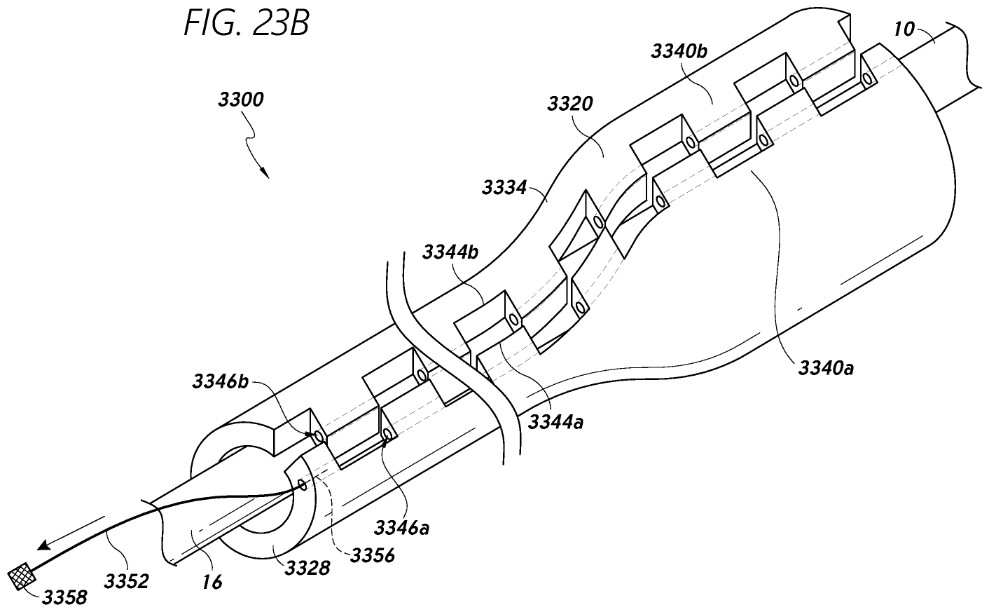

FIG. 23A is an exploded perspective view of an example of a peel-away catheter 3300 with an MCS system 10 preloaded therethrough, the peel-away catheter 3300 comprising a catheter shaft 3320 having a first interlocking portion 3340a and a second interlocking portion 3340b along a longitudinal portion. FIG. 23B is a perspective view of the catheter shaft 3320 of the peel-away catheter 3300 being peeled and/or removed from around the MCS system 10, including from around the shaft 16 of the MCS system 10. A shaft wall 3334 of the catheter shaft 3320 can comprise a pair of interlocking portions, such as the first and second interlocking portions 3340a, 3340b, extending along a longitudinal portion thereof. In some instances, an interior surface of the shaft wall 3334 can define a shaft delivery lumen 3322 extending through the catheter shaft 3320. FIG. 23A shows the catheter shaft 3320 in an unpeeled configuration, such as while the first interlocking portion 3340a and second interlocking portion 3340b are fitted and/or mated together. FIG. 23B shows the first and second interlocking portions 3340a, 3340b being unlocked and/or separated, such as to peel and/or remove the catheter shaft 3320 from around the MCS system 10.

The first and second interlocking portions 3340a, 3340b can extend along an entire or a portion of the longitudinal dimension, such as length, of the catheter shaft 3320, for example from a proximal end 3328 to a distal end 3330 of the catheter shaft 3320. For example, the first and second interlocking portions 3340a, 3340b can extend along an entire longitudinal dimension, such as length, of the shaft wall 3334. The first and second interlocking portions 3340a, 3340b can comprise a plurality of corresponding mating portions 3342a, 3342b configured to fit and/or mate with one another to form the interlock. For example, shapes of edges 3344a of the first interlocking portion 3340a and edges 3344b of the second interlocking portion 3340b can be configured to complement one another to form the fit and/or mate between the interlocking portions 3340a, 3340b. In some instances, mating edges 3344a, 3344b of the first and second interlocking portions 3340a, 3340b can each have a wave shape. In some instances, where each of the mating portions 3342a of the first interlocking portion 3340a comprises a crest of a wave shape, including a rectangular wave shape, and a corresponding mating portion 3342b of the second interlocking portion 3340b can comprise a trough of the wave shape, including the rectangular wave shape. Although the first and second interlocking portions 3340a, 3340b can comprise linear or substantially linear edges, it will be understood that any number of other shapes can also be applicable, including arcuate edges. For example, a first and second interlocking portion can comprise corresponding concave and convex portions configured to be fitted and/or mated together to form the interlock.

The peel-away catheter 3300 can comprise an elongate member 3350 comprising an elongate portion 3352 and a proximal elongate member tab 3358 associated with, such as coupled to, a proximal end 3354 of the elongate portion 3352. Referring to FIG. 23A, while the peel-away catheter 3300 is in the unpeeled configuration, at least a portion of the elongate portion 3352 can be slidably received through respective lumen portions extending through each mating portion 3342a, 3342b of the first and second interlocking portions 3340a, 3340b, respectively. In some instances, the first and second interlocking portions 3340a, 3340b of the catheter shaft 3320, such as the shaft wall 3334, can comprise corresponding elongate member lumen portions 3346a, 3346b (for example, extending through a longitudinal portion of the first and second interlocking portions 3340a, 3340b) to slidably receive respective portions of the elongate portion 3352. For example, respective portions of the shaft wall 3334 can define the corresponding elongate member lumen portion 3346a, 3346b extending at least partially through a longitudinal dimension of each mating portion 3342a, 3342b. In some instances, the first and second elongate member lumen portions 3346a, 3346b can follow a contour and/or profile of an interior and/or exterior surface of the shaft wall 3334. Each mating portion 3342a, 3342b can comprise a corresponding elongate member lumen portion 3346a, 3346b extending at least partially therethrough. For example, an elongate member lumen portion of the distal-most mating portion may or may not extend through an entire longitudinal dimension of the mating portion.

Referring to FIG. 23A, the elongate portion 3352 can be slidably disposed through the elongate member lumen portions 3346a, 3346b of the first and second interlocking portions 3340a, 3340b to maintain the catheter shaft 3320 in the unpeeled configuration. While the catheter shaft 3320 is in an unpeeled configuration, the elongate member 3350, including the elongate portion 3352, can be in a tension state to maintain the interlocking portions 3340a, 3340b in a mated (for example, interlocked) state. In some instances, a distal end 3356 of the elongate portion 3352 can be coupled to a portion of the shaft wall 3334 defining a portion of, including a distal end of, the distal-most elongate member lumen portion and/or a marker (not shown), including a radiopaque marker, comprising at least a portion disposed in the distal-most elongate member lumen portion. The distal end of the distal-most elongate member lumen portion can be proximal of or be at the distal end 3330 of the catheter shaft 3320. The distal-most elongate member lumen portion is shown as being on the first interlocking portion 3340a in FIG. 23A. The proximal end 3354 of the elongate portion 3352 can be disposed externally of the catheter shaft 3320, including proximally of a proximal end 3328 of the catheter shaft 3320, such as to facilitate manipulation of the elongate member 3350. For example, the distal end of the distal-most elongate member lumen portion can be proximal of or be at the distal end of the shaft wall 3334. The proximal end 3354 of the elongate portion 3352 can be disposed externally of the shaft wall 3334, including proximally of a proximal end of the shaft wall 3334.

Referring to FIG. 23B, the elongate member 3350 can be translated proximally to detach the distal end 3356 of the elongate portion 3352 from the shaft wall 3334 and/or marker to which it is coupled such that the elongate portion 3352 can be withdrawn through the elongate member lumen portions 3346a, 3346b and out of the catheter shaft 3320. An operator can engage the proximal elongate member tab 3358 to pull on the elongate portion 3352 to cause the elongate portion 3352 to translate proximally through the elongate member lumen portions 3346a, 3346b. FIG. 23B shows the distal end 3356 of the elongate portion 3352 being withdrawn through an elongate member lumen portion 3346a of the first interlocking portion 3340a. After the elongate portion 3352 is removed from the catheter shaft 3320, the interlocking portions 3340a, 3340b can unlock and/or separate to allow removal of the catheter shaft 3320 from around the MCS system 10. For example, the mating edges 3344a, 3344b of the first and second interlocking portions 3340a, 3340b can separate to allow the MCS system 10, including the shaft 16, to pass between the mating edges 3344a, 3344b.

It will be understood that although one elongate member is described with reference to FIGS. 23A and 23B, a plurality of elongate members can be used, such as to maintain corresponding pairs of a plurality of pairs of interlocking portions in the interlocked state. For example, the catheter shaft can be split and/or separated into a plurality of elongate shaft portions.

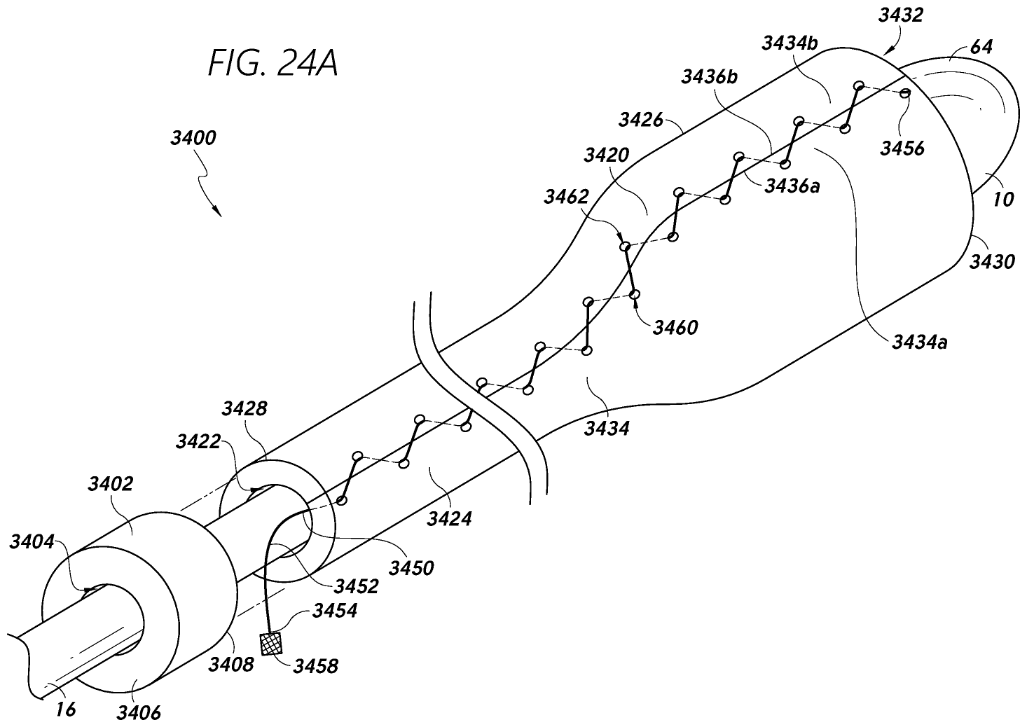

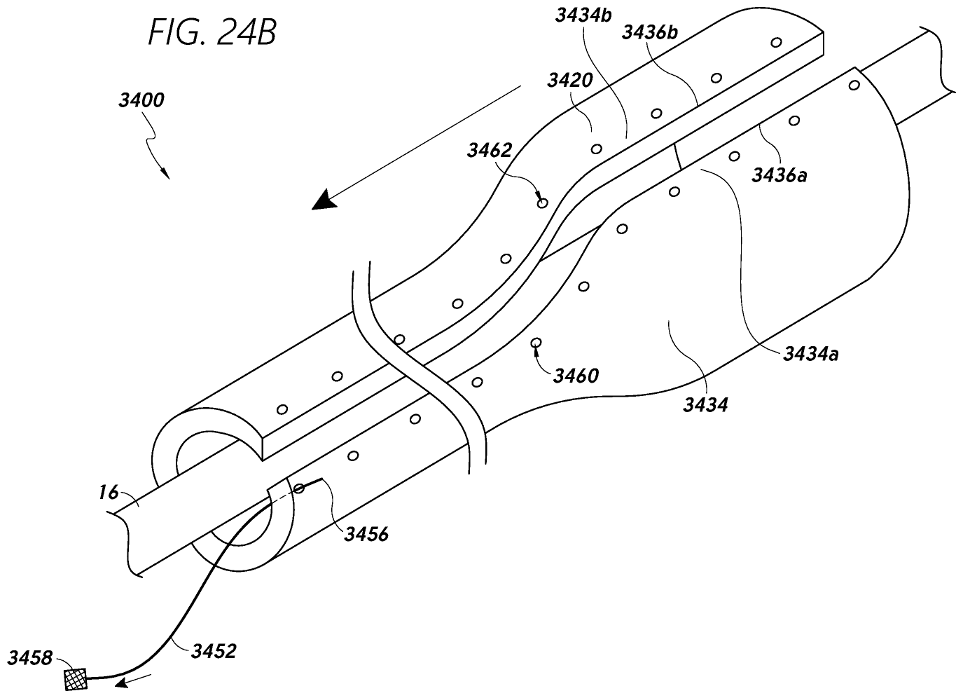

FIG. 24A is an exploded perspective view of an example of a peel-away catheter 3400 and an MCS system 10 preloaded through the peel-away catheter 3400. FIG. 24B is a perspective view of a catheter shaft 3420 of the peel-away catheter 3400 being peeled and/or removed from around the MCS system 10, including from around the shaft 16 of the MCS system 10. The peel-away catheter 3400 can comprise a catheter shaft 3420 and an elongate member 3450 configured to fasten together edges of a shaft wall 3434 of the catheter shaft 3420. FIG. 24A shows the catheter shaft 3420 in an unpeeled configuration, such as while edges of the shaft wall 3434 are fastened together by the elongate member 3450. FIG. 24B shows the elongate member 3450 being removed from the catheter shaft 3420 and the catheter shaft 3420 being peeled and/or removed from around the MCS system 10.

The elongate member 3450 can comprise an elongate portion 3452 and a proximal elongate member tab 3458 coupled to a proximal end 3454 of the elongate portion 3452. The shaft wall 3434 can have a first edge portion 3434a comprising a plurality of first openings 3460 at respective locations along a longitudinal portion of the first edge portion 3434a. The shaft wall 3434 can have a second edge portion 3434b comprising a plurality of second openings 3462 at respective locations along a longitudinal portion of the second edge portion 3434b. For example, the plurality of first and second openings 3460, 3462 can extend through a thickness of the shaft wall 3434. In some instances, an interior surface of the shaft wall 3434 can define a shaft delivery lumen 3422 extending through the catheter shaft 3420.

The elongate portion 3452 can be configured to be disposed through the plurality of openings 3460, 3462. In some examples, as shown in FIG. 24A, the elongate portion 3452 can extend through the openings 3460, 3462 in an alternating fashion. For example, the elongate portion 3452 can be configured to be disposed alternatingly (e.g., extend through) between openings 3460 on the first edge portion 3434a and openings 3462 on the second edge portion 3434b, such that the elongate portion 3452 can fasten together the first and second edge portions 3434a, 3434b. For example, the elongate member 3450 can suture, stitch and/or lace together the first and second edge portions 3434a, 3434b. A distal end 3456 of the elongate portion 3452 can be coupled to a distal portion 3426 of the catheter shaft 3420. For example, the distal end 3456 of the elongate portion 3452 can be coupled to a portion of the shaft wall 3434, including a portion of the shaft wall 3434 defining at least a portion of the distal-most one of the openings 3460, 3462 of the first or second edge portions 3434a, 3434b. In some instances, tension can be maintained in the elongate portion 3452 disposed (e.g., extending) through the plurality of first and second openings 3460, 3462, so as to facilitate maintaining a first edge 3436a of the first edge portion 3434a adjacent and in contact with a second edge 3436b of the second edge portion 3434b. In some instances, maintaining tension in the elongate portion 3452 can comprise fixing and/or coupling one or more portions of the elongate portion 3452 to a respective portion of the shaft wall 3434, including a portion of the shaft wall 3434 defining one or more of the plurality of first and second openings 3460, 3462. The proximal elongate member tab 3458 can be disposed externally, including proximally of a proximal end 3428, of the catheter shaft 3420, so as to facilitate manipulation by an operator. For example, the proximal elongate member tab 3458 can be disposed externally, including proximally of a proximal end of the shaft wall 3434.

Referring to FIG. 24B, an operator can engage the proximal elongate member tab 3458 to pull on the elongate portion 3452 to cause the elongate portion 3452 to translate proximally through the first and second openings 3460, 3462. The elongate portion 3452 can be removed from the shaft wall 3434 to facilitate splitting and/or separation of the shaft wall 3434 along the first and second edges 3436a, 3436b. For example, the operator can pull on the proximal elongate member tab 3458 to decouple and/or disconnect the distal end 3456 of the elongate portion 3452 from the shaft wall 3434, and/or any coupling of the elongate portion 3452 to any other portion of the shaft wall 3434, to allow proximal translation of the elongate portion 3452. After the elongate portion 3452 is removed from the catheter shaft 3420, the first and second edges 3436a, 3436b can split and/or separate to allow removal or separation of the shaft 3420 from around the MCS system 10, including the shaft 16. FIG. 24B shows the distal end 3456 of the elongate portion 3452 being withdrawn through a first opening 3460 of the first edge portion 3434a.

It will be understood that although one elongate member is described with reference to FIGS. 24A and 24B, a plurality of elongate members can be used, such as to maintain corresponding pairs of a plurality of pairs of interlocking portions in the interlocked state. For example, the catheter shaft can be split and/or separated into a plurality of elongate shaft portions.

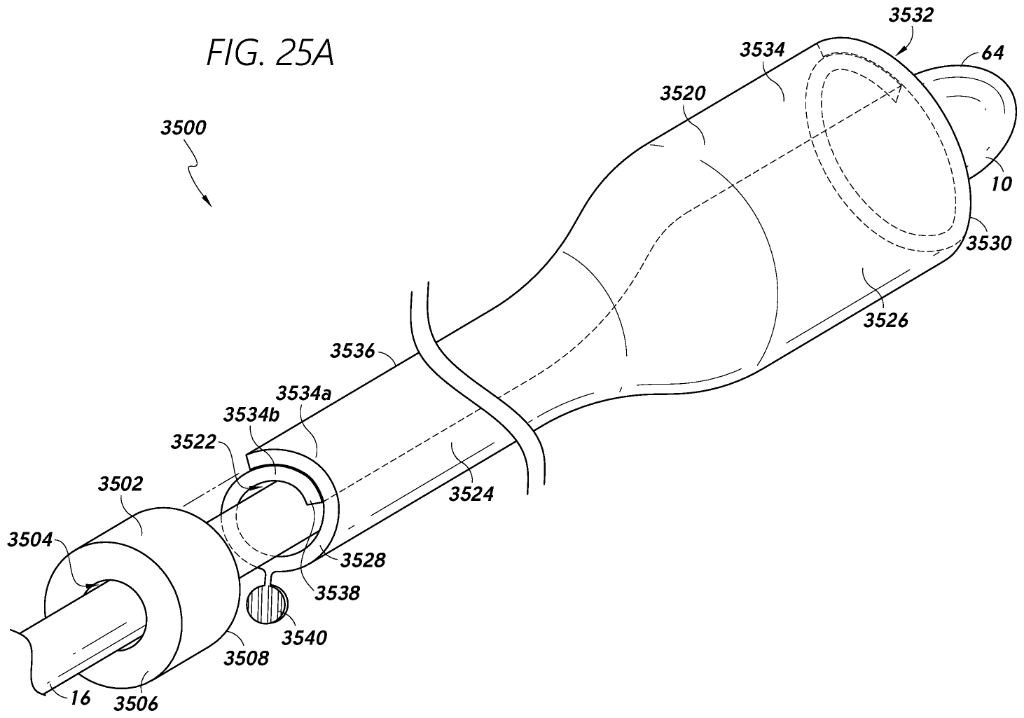

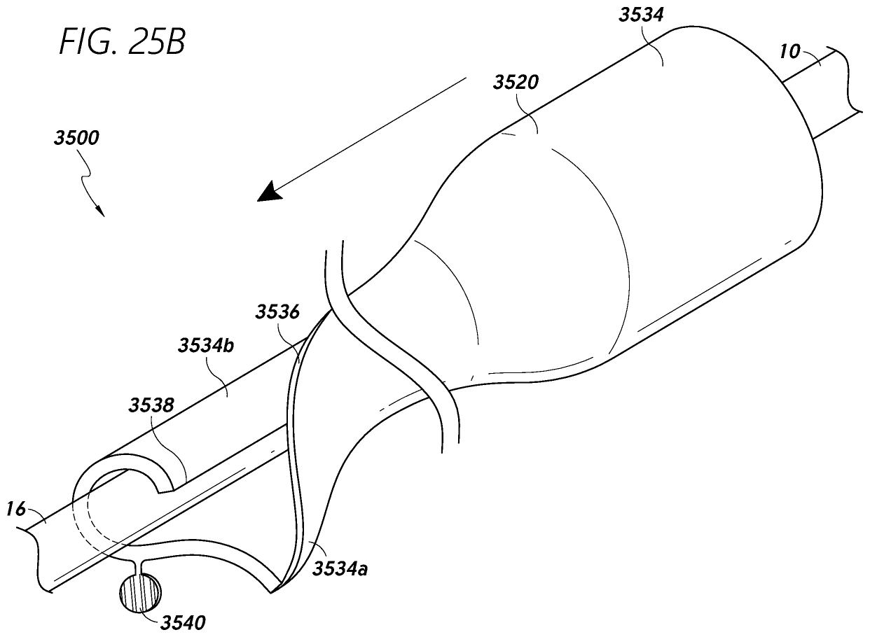

FIG. 25A is an exploded perspective view of an example of a peel-away catheter 3500 and an MCS system 10 preloaded through the peel-away catheter 3500, the peel-away catheter 3500 comprising a catheter shaft 3520 having a rolled configuration while in an unpeeled configuration. FIG. 25B is a perspective view of a catheter shaft 3520 of the peel-away catheter 3500 being peeled and/or removed from around the MCS system 10, including from around the shaft 16 of the MCS system 10. For example, a shaft wall 3534 of the catheter shaft 3520 can comprise a first overlapping wall portion 3534a configured to be over and/or in contact with a second overlapping wall portion 3534b along a longitudinal portion of the shaft wall 3534. In some instances, portions of an interior surface of the shaft wall 3534 can define a shaft delivery lumen 3522 extending through the catheter shaft 3520. FIG. 25A shows the catheter shaft 3520 in an unpeeled configuration, such as while the catheter shaft 3520 is in the rolled configuration. FIG. 25B shows the catheter shaft 3520 being unrolled, such as to peel and/or remove the catheter shaft 3520 from around the MCS system 10.

Referring to FIG. 25A, while in the unpeeled configuration, the catheter shaft 3520 can assume a rolled configuration. The first overlapping wall portion 3534a can be over and/or in contact with the second overlapping wall portion 3534b along an entire longitudinal dimension, such as length, of the shaft wall 3534. For example, a first longitudinal edge 3536 of the shaft wall 3534 can be over and/or in contact with an exterior surface portion of the second overlapping wall portion 3534b. A second longitudinal edge 3538 of shaft wall 3534 can be over and/or in contact with an interior surface of the first overlapping wall portion 3534a. In some instances, a degree of overlap of the shaft wall 3534 can be less than 100% of a circumference of the catheter shaft 3520. In some instances, the degree of overlap of the shaft wall 3534 can be between about 20% to about 80% of the circumference of the catheter shaft 3520, including about 40% to about 60%. The degree of overlap of the shaft wall 3534 can be selected to facilitate maintaining the catheter shaft 3520 around the MCS system 10 while the catheter shaft 3520 is in the rolled configuration, while allowing removal of the catheter shaft 3520 from around the MCS system 10 without or substantially without damaging the MCS system 10 and/or injuring the patient.

The peel-away catheter 3500 can comprise a proximal shaft tab 3540 coupled to a proximal portion 3524 of the catheter shaft 3520, including a proximal end 3528 of the catheter shaft 3520. In some instances, the proximal shaft tab can be coupled a proximal portion, including a proximal end, of the shaft wall 3534. In some instances, the proximal shaft tab 3540 can be coupled at a position circumferentially spaced away from the first and second overlapping wall portions 3534a, 3534b. In some instances, the proximal shaft tab 3540 can be opposingly oriented around the circumference of the catheter shaft 3520 relative to the first and second overlapping wall portions 3534a, 3534b.

Referring to FIG. 25B, an operator can engage the proximal shaft tab 3540 to peel away and/or remove the catheter shaft 3520 from around the MCS system 10. For example, the proximal shaft tab 3540 can be pulled laterally away from a longitudinal axis of the catheter shaft 3520 so as to peel away and/or remove the catheter shaft 3520 from around the MCS system 10.

In some alternative instances, a catheter shaft can comprise no portion that overlaps while the catheter shaft is in the rolled configuration. In some instances, a shaft wall of the catheter shaft can comprise edges configured to be adjacent to one another while the catheter shaft is in an unpeeled configuration. For example, the shaft wall can assume a “C-shape.”

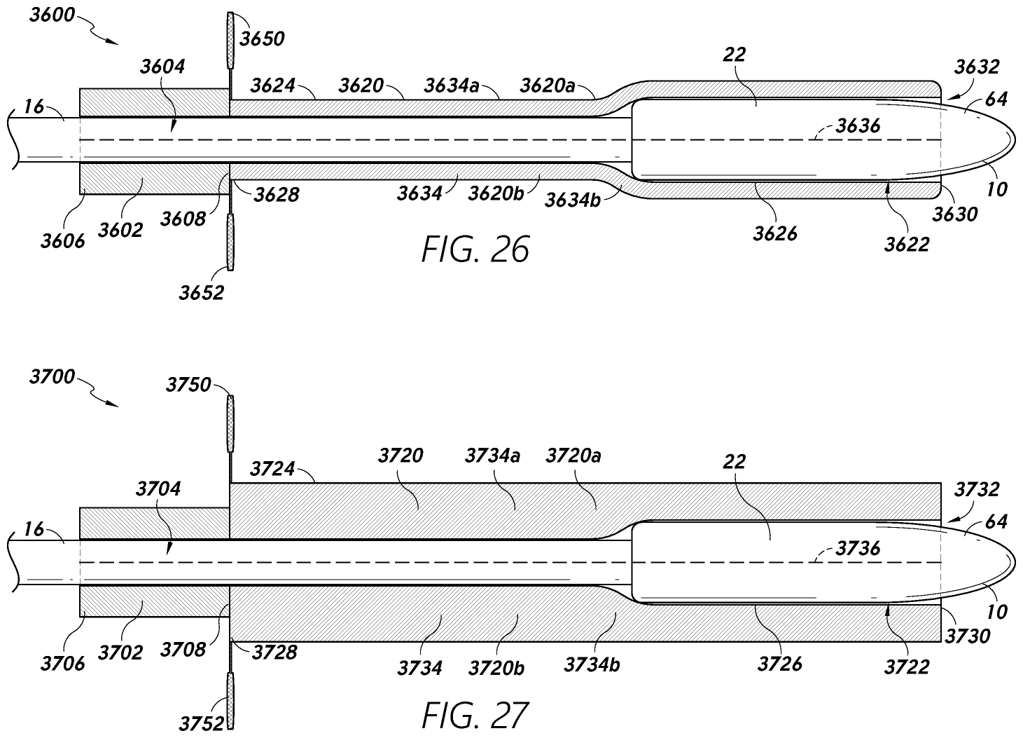

FIGS. 26 and 27 are side cross-sectional views of examples of peel-away catheters 3600, 3700 having an MCS system 10 preloaded therethrough. The peel-away catheters 3600, 3700 can each comprise a shaft 3620, 3720 with an outer diameter that varies along a longitudinal dimension and an outer diameter that is the same or similar along the longitudinal dimension, respectively. Referring to FIG. 26, the catheter shaft 3620 can comprise a proximal portion 3624 having an outer diameter smaller than that of a distal portion 3626 of the catheter shaft 3620. The outer diameter of the distal portion 3626 can be larger than that of the proximal portion 3624 to accommodate a pump 22 of the MCS system 10, which has a larger diameter than a shaft 16 that is proximal of the pump 22. For example, the outer profile, contour and/or shape of the catheter shaft 3620 can correspond to, conform to and/or match that of the portion of the MCS system 10 preloaded through the catheter shaft 3620. Referring to FIG. 27, the catheter shaft 3720 can comprise an outer diameter that is the same or substantially the same along an entire or substantially entire length of the catheter shaft 3720. As shown in FIGS. 26 and 27, catheter shafts 3620, 3720 can comprise shaft delivery lumens 3622, 3722, respectively, extending therethrough. In some instances, a respective diameter of the shaft delivery lumen 3622, 3722 can correspond to, conform to and/or match that of the portion of the MCS system 10 preloaded through the catheter shaft 3620, 3720. For example, a respective diameter of the shaft delivery lumen 3622, 3722 can be smaller at a proximal portion 3624, 3724 than a distal portion 3626, 3726 of the catheter shafts 3620, 3720. The diameter of the shaft delivery lumen 3622, 3722 extending through the distal portion 3626, 3726 can be configured to accommodate the pump 22 of the MCS system 10. Alternatively, a diameter of a shaft delivery lumen can remain the same or similar along an entire or substantially entire length of the catheter shaft 3620, 3720.

The peel-away catheters 3600, 3700 can comprise first proximal shaft tabs 3650, 3750 coupled to proximal portions 3624, 3724 of the catheter shafts 3620, 3720, respectively. The peel-away catheters 3600, 3700 can comprise second proximal shaft tabs 3652, 3752 coupled to proximal portions 3624, 3724 of the catheter shafts 3620, 3720, respectively. For example, each of the first proximal shaft tabs 3650, 3750 can be coupled to a proximal portion of a respective first elongate shaft portion 3620a, 3720a. The second proximal shaft tab 3652, 3752 can be coupled to a proximal portion of a respective second elongate shaft portion 3620b, 3720b. Pulling the first and second proximal shaft tabs 3650, 3652 laterally away from a longitudinal axis of the catheter shaft 3620 can cause the catheter shaft 3620 to separate into the first and second elongate shaft portions 3620a, 3620b. Pulling the first and second proximal shaft tabs 3750, 3752 laterally away from a longitudinal axis of the catheter shaft 3720 can cause the catheter shaft 3720 to separate into the first and second elongate shaft portions 3720a, 3720b.

The catheter shafts 3620, 3720 can each comprise a first weakened longitudinal portion 3636, 3736 and a second weakened longitudinal portion 3638 (not shown), 3738 (not shown). The first weakened longitudinal portion 3636, 3736 and second weakened longitudinal portion 3638, 3738 can extend along at least a portion of a longitudinal dimension of the catheter shaft 3620, 3720, including an entire or substantially entire length of the catheter shaft 3620, 3720. In some instances, the first weakened longitudinal portion 3636, 3736 and second weakened longitudinal portion 3638, 3738 can extend from a proximal end 3628, 3728 to a distal end 3630, 3730 of the catheter shaft 3620, 3720. For example, a shaft wall 3634, 3734 of each catheter shaft 3620, 3720 can comprise the first weakened longitudinal portion 3636, 3736 and second weakened longitudinal portion 3638, 3738 along an entire or substantially entire length thereof. In some instances, the first weakened longitudinal portion 3636, 3736 and/or second weakened longitudinal portion 3638, 3738 can comprise indentations and/or perforations formed in the shaft wall 3634, 3734. In some instances, at least a portion of an interior surface of the shaft wall 3634, 3734 can define a shaft delivery lumen 3622, 3722 configured to slidably receive the MCS system 10. In some instances, the first weakened longitudinal portion 3636, 3736 and second weakened longitudinal portion 3638, 3738 can be opposingly oriented around a circumference of the respective catheter shaft 3620, 3720. An operator can pull the first and second proximal shaft tabs 3650, 3652 laterally away from a longitudinal axis of the catheter shaft 3620 can cause the shaft wall 3634 to separate into the first and second elongate shaft wall portions 3634a, 3634b. The operator can pull the first and second proximal shaft tabs 3750, 3752 laterally away from a longitudinal axis of the catheter shaft 3720 can cause the shaft wall 3734 to separate into the first and second elongate shaft wall portions 3734a, 3734b.

It will be understood that although FIGS. 26 and 27 describe separating the catheter shafts 3620, 3720 into two elongate shaft portions, the catheter shafts 3620, 3720 can be separated into more elongate shaft portions. For example, the catheter shafts 3620, 3720 can comprise a number of weakened longitudinal portions configured to allow the catheter shafts 3620, 3720 to separate into the desired number of elongate shaft portions.

In some instances, the catheter shafts 3120, 3220, 3320, 3420, 3520, 3620, 3720 described with reference to FIGS. 21A through 27 can have a longitudinal dimension, such as length, configured to receive the MCS system 10. For example, while the MCS system 10 is preloaded, a distal tip 64, such as a nosecone portion, of the MCS system 10 is disposed distally of a distal end 3130, 3230, 3330, 3430, 3530, 3630, 3730 of the catheter shaft 3120, 3220, 3320, 3420, 3520, 3620, 3720 through a distal opening 3132, 3232, 3332, 3432, 3532, 3632, 3732.

In some instances, the catheter shafts 3120, 3220, 3320, 3420, 3520 described with reference to FIGS. 21A through 25B can comprise an outer diameter and/or a diameter of a shaft delivery lumen 3122, 3222, 3322, 3422, 3522, 3622, 3722 configured to correspond, conform and/or match that of the portion of the MCS system 10 disposed therethrough. For example, the outer diameter and/or a diameter of the shaft delivery lumen 3122, 3222, 3322, 3422, 3522 of a proximal portion 3124, 3224, 3324, 3424, 3524 can be smaller than that of a distal portion 3126, 3226, 3326, 3426, 3526. For example, the distal portion 3126, 3226, 3326, 3426, 3526 can be sized to accommodate a pump 22 of the MCS system 100. Alternatively, one or both of the outer diameter and/or diameter of the shaft delivery lumen 3122, 3222, 3322, 3422, 3522 can be the same or similar along an entire or substantially entire length of the catheter shaft 3120, 3220, 3320, 3420, 3520.

The peel-away catheters 3200, 3300, 3400, 3500, 3600, 3700 described with reference to FIGS. 22A through 27 can each comprise catheter hub 3202, 3302, 3402, 3502, 3602, 3702. The catheter shafts 3220, 3320, 3420, 3520, 3620, 3720 described with reference to FIGS. 22A through 27 can extend distally from the corresponding catheter hub 3202, 3302, 3402, 3502, 3602, 3702. The catheter hubs 3202, 3302, 3402, 3502, 3602, 3702 can have one or more features of the catheter hub 3102 described with reference to FIGS. 21A and 21B. For example, the catheter hubs 3202, 3302, 3402, 3502, 3602, 3702 can comprise a hub delivery lumen 3204, 3304, 3404, 3504, 3604, 3704 extending therethrough, such as from a proximal end 3206, 3306, 3406, 3506, 3606, 3706 to a distal end 3208, 3308, 3408, 3508, 3608, 3708. In some instances, the catheter hub 3202, 3302, 3402, 3502, 3602, 3702 is not a peel-away hub. For example, after the peel-away catheter 3200, 3300, 3400, 3500, 3600, 3700 carrying the MCS system 10 is advanced to a target location, the catheter hub 3202, 3302, 3402, 3502, 3602, 3702 can be separated from the respective catheter shaft 3220, 3320, 3420, 3520, 3620, 3720 such that the catheter shaft 3220, 3320, 3420, 3520, 3620, 3720 can be remove from around the MCS system 10. Alternatively, the catheter hub 3202, 3302, 3402, 3502, 3602, 3702 can be peelable and can be removed together with or separately from the catheter shaft 3220, 3320, 3420, 3520, 3620, 3720.

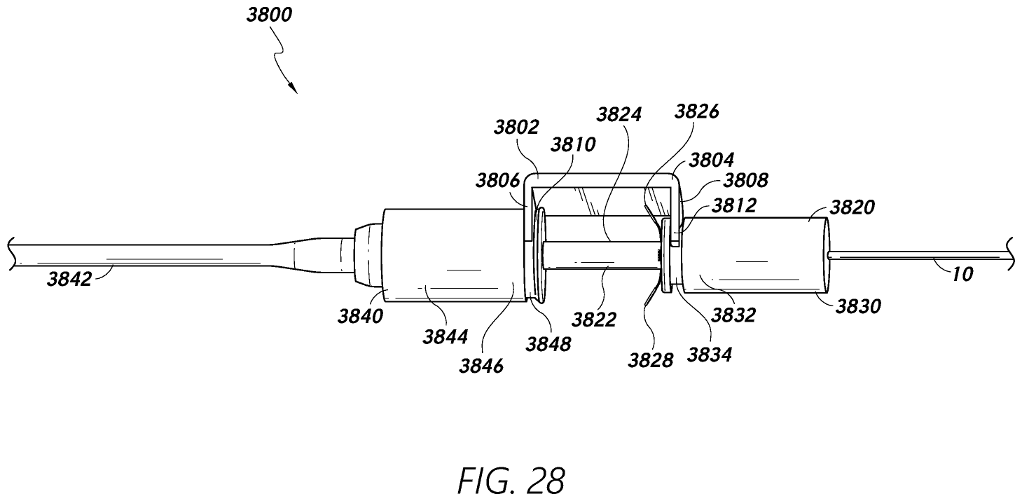

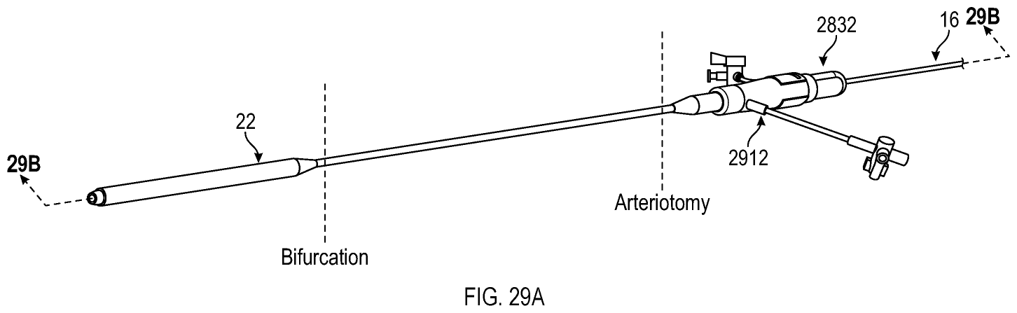

FIG. 28 is a side view of a delivery system 3800 having an MCS system 10 preloaded therethrough. The delivery system 3800 can comprise a peel-away catheter 3820 comprising a catheter shaft 3822 extending distally from a catheter hub 3830, and an introducer sheath 3840 comprising an introducer sheath shaft 3842 extending distally from an introducer sheath hub 3844. The MCS system 10 can be preloaded through the peel-away catheter 3820. A portion of the catheter shaft 3822 carrying the MCS system 10 can be disposed through the introducer sheath 3840, such as a delivery lumen of the introducer sheath 3840. The introducer sheath 3840 can have one or more features of the introducer sheaths described herein. In some instances, the introducer sheath 3840 can be an expandable introducer sheath. The delivery system 3800 can comprise a spacer 3802 configured to axially space the introducer sheath hub 3844 from the catheter hub 3830. In some instances, a position of the MCS system 10 can be fixed relative to the peel-away catheter 3820. For example, the catheter hub 3830 can comprise a frictional engagement member configured to engage with a portion of the MCS system 10, such as a portion of the shaft 16, disposed through the hub delivery lumen to fix a translational position of the MCS system 10.

The spacer 3802 can comprise one or more features of the spacer 2002 described with reference to FIG. 20. For example, the spacer 3802 can comprise at least a portion configured to be positioned between the introducer sheath hub 3844 and the peel-away catheter hub 3830 to axially space the introducer sheath hub 3844 from the catheter hub 3830. The spacer 3802 can be configured to maintain a fixed distance between the introducer sheath hub 3844 and the catheter hub 3830, for example such that the introducer sheath hub 3844 is distal of and aligned with the catheter hub 3830 along a longitudinal axis of the introducer sheath hub 3844 and/or the catheter hub 3830. In some instances, after the introducer sheath 3840 is navigated to a target position, such as while carrying a dilator extending therethrough, a peel-away catheter 3820 can be advanced into the introducer sheath 3840. For example, the dilator can be withdrawn and the peel-away catheter 3820 having the MCS system 10 preloaded therethrough can be advanced at least partially through the introducer sheath 3840. The catheter hub 3830 can comprise an engagement feature to engage with a portion of the MCS system 10 extending through the catheter hub 3830 to maintain an axial and/or rotational position of the MCS system 10. In some instances, the spacer 3802 can be configured to maintain spacing and/or alignment between the introducer sheath hub 3844 and the catheter hub 3830 after the peel-away catheter 3820 is desirably positioned. For example, spacing and/or alignment can be maintained while the MCS system 10 is deployed from the peel-away catheter 3820. The spacer 3802 can be removed to facilitate peeling away and/or removal of the peel-away shaft 3822, such as after the MCS system 10 is deployed from the peel-away catheter 3820. In some instances, the catheter hub 3830 can be re-engaged with the MCS system 10 after removal of the catheter shaft 3822, such that re-engaging the spacer 3802 with the introducer sheath hub 3844 and the catheter hub 3830 can facilitate fixing a position of the MCS system 10 relative to the introducer sheath 3840.

The spacer 3802 can comprise a distal portion 3806 configured to engage with the introducer sheath hub 3844 and a proximal portion 3808 configured to engage with the catheter hub 3830. A medial portion 3804 can extend between and couple the proximal portion 3808 and distal portion 3806. The distal portion 3806 can comprise a first hub engagement feature 3810 configured to engage the introducer sheath hub 3844, including a corresponding spacer engagement feature 3848 of the introducer sheath hub 3844. The proximal portion 3808 can comprise a second hub engagement feature 3812 configured to engage with the catheter hub 3830, including a corresponding spacer engagement feature 3834 of the catheter hub 3830. In some instances, the spacer engagement feature 3848 can be on a proximal portion 3846 of the introducer sheath hub 3844. In some instances, the spacer engagement feature 3834 can be on a distal portion 3832 of the catheter hub 3830.

In some instances, the spacer engagement feature 3848 of each of the introducer sheath hub 3844 and the catheter hub 3830 can comprise a groove and/or recess extending at least partially circumferentially around, including around an entire circumference of, a portion of the respective hub. The first and second hub engagement features 3810, 3812 can comprise at least a portion configured to be received by and/or within the respective groove and/or recess to mate and/or couple with the groove and/or recess. In some instances, at least a portion of the distal portion 3806 and proximal portion 3808 can be oriented at an angle relative to, including being perpendicular or substantially perpendicular to, the medial portion 3804. In some instances, while the spacer 3802 is engaged with the catheter hub 3830 and the introducer sheath hub 3844, at least a portion of the medial portion 3804 can be oriented along a direction parallel or substantially parallel to the longitudinal axis of the catheter hub 3830 and/or the introducer sheath hub 3844. At least a portion of each of the distal portion 3806 and the proximal portion 3808 can be received within a respective groove and/or recess of the introducer sheath hub 3844 or catheter hub 3830. The first hub engagement feature 3810 can comprise the edge of the distal portion 3806 received within the groove and/or recess of the introducer sheath hub 3844. The second hub engagement feature 3812 can comprise the edge of the proximal portion 3808 received in the groove and/or recess of the catheter hub 3830. The edges of the distal portion 3806 and proximal portion 3808 can be configured to have complement shape and/or size of the respective groove and/or recess. For example, the edge of the distal portion 3806 and/or proximal portion 3808 received within the groove and/or recess of the introducer sheath hub 3844 and/or catheter hub 3830 can have a concave curvature configured to mate with a groove and/or recess extending along a path having a convex shape. Alternatively or in combination, the spacer engagement features can have any number of other different configurations, including slots, indentations, etc.

In some instances, the catheter shaft 3822 can comprise one or more features of the catheter shaft 3620 described with reference to FIG. 26. For example, a first proximal shaft tab 3826 and a second proximal shaft tab 3828 can be coupled to respective portions of a proximal portion 3824 of the catheter shaft 3822. An operator can pull the first proximal shaft tab 3826 and the second proximal shaft tab 3828 laterally away from a longitudinal axis of the catheter shaft 3822 so as to separate the catheter shaft 3822 into first and second elongate shaft portions.

In some instances, a medical delivery system can comprise one or more other catheter shafts described herein. For example, the catheter shafts 3120, 3220, 3320, 3420, 3520, 3620 3720 described with reference to FIGS. 21A through 27 can be applicable. In some instances, the catheter shafts 3120, 3220, 3320, 3420, 3520, 3620 3720 can each be coupled to a catheter hub having one or more features of the catheter hub 3830, including the spacer engagement features.

It will be understood that one or more of the catheter shafts 3120, 3220, 3320, 3420, 3520, 3620 3720 described herein can comprise one or more markers coupled thereto and/or formed therein, including radiopaque markers, to facilitate visualizing a position of the catheter shaft. In some instances, the markers can be on and/or coupled to the exterior surface of the catheter shaft 3120, 3220, 3320, 3420, 3520, 3620 3720. For example, the catheter shaft 3120, 3220, 3320, 3420, 3520, 3620 3720 can comprise a plurality of radiopaque markers arranged at respective positions along a longitudinal dimension to facilitate visualizing advancement of the catheter shaft 3120, 3220, 3320, 3420, 3520, 3620 3720 through an introducer sheath. In some instances, one or more markers can be on the distal portion 3126, 3226, 3326, 3426, 3526, 3626 3726, including at or proximate to the distal end 3130, 3230, 3330, 3430, 3530, 3630 3730, of the catheter shaft 3120, 3220, 3320, 3420, 3520, 3620 3720. In some instances, one or more elongate members described herein, including one or more of the elongate portions of the elongate members, can comprise one or more markers, such as radiopaque markers, coupled thereto and/or formed therein. In some instances, one or more of the elongate portions, such as distal portions and/or distal ends thereof, can comprise one or more radiopaque markers coupled thereto and/or formed therein. The markers, including the radiopaque markers, can provide visual indication to an operator relating to the position of the catheter shafts, including a position of a distal portion of a catheter shaft. For example, the markers can reduce or prevent over insertion of the catheter shaft, such as to prevent positioning a distal end of the catheter shaft distally of a distal end of the introducer sheath during advancement of the catheter shaft and/or MCS system.

In some alternative instances, a peel-away catheter can comprise a catheter shaft that is partially peelable. For example, a proximal portion of the catheter shaft can remain around an MCS system after more distal portions of the catheter shaft are peeled and/or removed from around the MCS system. For example, more distal portions of the catheter shaft can comprise one or more features of the catheter shafts described herein. In some instances, the peel-away catheter can comprise a catheter hub that is not peelable and a catheter shaft with a proximal portion that is not peelable. As described herein, a peel-away catheter can be inserted into an introducer sheath for delivery of the MCS system. In some instances, the proximal portion of the catheter shaft can be decoupled, separated and/or disengaged from the distal peelable portions of the catheter shaft. The portions of the catheter shaft not peeled and/or removed can be sized such that the unpeeled portions remain within an introducer sheath hub of the introducer sheath, such as having a length shorter than that of the introducer sheath hub. In some alternative instances, a catheter shaft can comprise a distal portion configured to accommodate a pump of the MCS system that is peelable, and a proximal portion that is not peelable. For example, portions of the catheter shaft proximal of the portion configured to accommodate the pump can remain around the MCS system. In some instances, portions of the catheter shaft that is not peeled can be received in the introducer sheath.

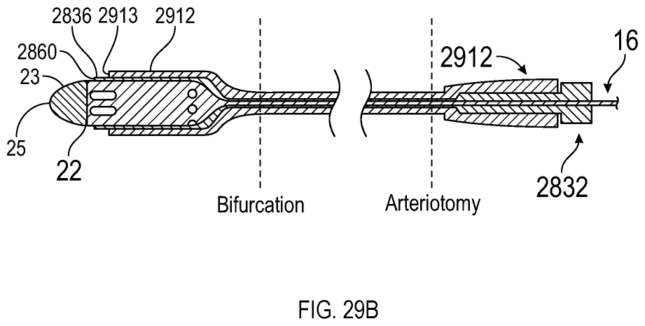

FIGS. 29A and 29B are perspective and cross-section views respectively and illustrate the MCS device 22 distally of the bifurcation placed within an enlarged portion of the insertion catheter 2832 and inserted within the introducer sheath 2912 prior to deployment and advancement of the MCS device 22 farther distally into the aorta. FIG. 29B is a truncated schematic of the system illustrating the various relative locations of various components. As shown in FIGS. 29A and 29B, in this embodiment, the enlarged portion of the insertion catheter 2832 containing the MCS device 22 may not be located entirely distally of a distal end 2913 of the introducer sheath 2912, as further described.

As shown, the introducer sheath 2912 is relatively longer than in other embodiments described herein and thus may extend into the aorta beyond the bifurcation. Additionally, the tubular body 2836 may be relatively shorter than as depicted in FIG. 18. Lengthening the introducer sheath 2912 and shortening the tubular body 2836 may result in the enlarged portion of the insertion catheter being fully contained in the introducer sheath 2912 except for a small distal portion 2860 extending out the distal end of the introducer sheath 2912. In some embodiments, when the insertion catheter 2832 fully inserted into the introducer sheath 2912 and prior to advancement of the MCS device 22 father into the aorta, the MCS device 22 may be fully contained within the introducer sheath 2912 and not extend out distally therefrom. As shown, a distal end 2860 of the tubular body 2836 of the insertion catheter 2832 may be proximal of a distal end 25 of the distal portion 23 of the MCS device 22. The distal end 25 of the MCS device 22 may extend distally beyond the distal end 2860 of the introducer sheath 2912 by no more than 0.5 cm, no more than 1 cm, no more than 1.5 cm, no more than 2 cm, no more than 3 cm, or by about 1 cm.

The distal end 2860 of the tubular body 2836 of the insertion catheter 2832 may be located distally of the distal end 2913 of the introducer sheath 2912, as shown. In some embodiments, the distal end 2860 may be coterminous with, or proximal of, the distal end 2913. The distal end 2860 of the tubular body 2836 may be distally or proximally offset from the distal end 2913 of the introducer sheath 2912 by no more than 0.5 cm, no more than 1 cm, no more than 2 cm, no more than 3 cm, no more than 4 cm, no more than 5 cm, or no more than 10 cm. When the insertion catheter 2832 is fully docked with the introducer sheath 2912, the tubular body 2836 may not be visible or exposed, as the tubular body 2836 may be contained within the introducer sheath 2912.

Various advantages result from the described configurations of FIGS. 29A and 29B. For example, such configurations with a relatively shorter tubular body 2836 may be easier to handle, as less of the tubular body 2836 is required to be advanced into the body. The shorter tubular body 2836 may have increased stiffness and thus increased responsiveness to pushing forces. Additionally, because less length of the tubular body 2836 is advanced into the body and the aorta, the risk of internal traumatic injury is reduced.

Further, upon removal of the tubular body 2836 and insertion catheter 2832 from the introducer sheath 2912 at the end of the procedure, there is no need to pull the enlarged portion of the insertion catheter 2832 proximally back into the introducer sheath 2912, like in other embodiments, such as that shown in FIG. 18. Instead, the enlarged portion of the insertion catheter 2832 is already within the introducer sheath 2912 which eliminates the additional force required to open the introducer sheath 2912 and pull the enlarged portion of the insertion catheter 2832 proximally back inside. This reduces the risk of the introducer sheath 2912 from buckling or causing withdrawal resistance and other difficulties for the operator.

When the insertion catheter 2832 remains inside the introducer sheath 2912, as previously described and depicted in FIG. 29B, the introducer sheath 2912 may have a larger diameter than other embodiments described herein, such as embodiments depicted in FIG. 18 with the MCS device 22 fully extended through the introducer sheath 2912. Such configurations with a larger diameter do not create an increased risk of occlusion since the placement of the distal end 23 is distal to the bifurcation, as depicted in FIG. 29A.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated. The word “about” may refer to values within ±1%, ±2%, ±3%, ±4%, ±5%, ±10%, ±15%, or other ranges depending on context and as may be understood by one of ordinary skill in the art.

### EXAMPLE EMBODIMENTS

The following are non-limiting example embodiments of some of the subject matter described herein.

Example 1: An insertion catheter for a circulatory support catheter having a circulatory support device carried by an elongate flexible catheter shaft, the insertion catheter comprising: a tubular body having a proximal end, a distal end, a longitudinal axis extending between the proximal and distal ends, a proximal portion adjacent the proximal end, a distal portion adjacent the distal end, and a mid portion between the proximal portion and the distal portion, wherein the distal portion of the tubular body is configured to axially movably receive the circulatory support device, and wherein a diameter of the distal portion is greater than a diameter of the mid portion.

Example 2: The insertion catheter of any example herein, in particular Example 1, further comprising a hub having a proximal end and a distal end, wherein the distal end of the hub fluidly connects with the proximal end of the tubular body.

Example 3: The insertion catheter of any example herein, wherein the tubular body further comprises a proximal transition section between the proximal portion and the mid portion and a distal transition section between the distal portion and the mid portion.

Example 4: The insertion catheter of any example herein, in particular Example 3, wherein the distal portion and the distal transition section are configured to receive the circulatory support device and the mid portion, proximal transition section, proximal portion, and hub are configured to receive the elongate flexible catheter shaft.

Example 5: The insertion catheter of any example herein, wherein the distal portion, mid portion, and proximal portion are concentric about the longitudinal axis.

Example 6: The insertion catheter of any example herein, wherein the mid portion comprises a smaller outer diameter than an outer diameter of the proximal portion and an outer diameter of the distal portion.

Example 7: The insertion catheter of any example herein, wherein the distal portion comprises a larger inner diameter than an inner diameter of the mid portion and the proximal portion.

Example 8: The insertion catheter of any example herein, wherein the tubular body is configured so that the proximal portion remains outside the body of a patient when the insertion catheter is in use.

Example 9: The insertion catheter of any example herein, wherein the tubular body is configured so that the mid portion extends from outside the body of a patient through an arteriotomy of a femoral artery, through the femoral artery, through a bifurcation of the femoral artery where it joins an aorta, and into the aorta when the insertion catheter is in use.

Example 10: The insertion catheter of any example herein, wherein the tubular body is configured so that the distal portion is positioned within an aorta of a patient when the insertion catheter is in use.

Example 11: The insertion catheter of any example herein, in particular Examples 2 to 10, wherein the distal end of the tubular body is configured to extend past a distal end of an introducer sheath when the insertion catheter is fully docked with the introducer sheath when in use.

Example 12: The insertion catheter of any example herein, in particular Example 11, in parti wherein the tubular body of the insertion catheter has sufficient collapse resistance to maintain patency when passed through one or more hemostatic valves of the introducer sheath.

Example 13: The insertion catheter of any example herein, in particular Example 11, wherein the distal end of the hub comprises one or more features for preventing rotational and/or axial movement of the insertion catheter when docked with the introducer sheath.

Example 14: The insertion catheter of any example herein, wherein the insertion catheter comprises a tube with a valve in fluid communication with an inner lumen of the tubular body of the insertion catheter configured for flushing with saline.

Example 15: The insertion catheter of any example herein, wherein the insertion catheter comprises a hemostatic valve.

Example 16: The insertion catheter of any example herein, wherein the insertion catheter comprises a plug disposed at the proximal end of the hub configured to connect to a sterile shield sleeve.

Example 17: The insertion catheter of any example herein, wherein the elongate flexible catheter shaft comprises a visual marker spaced proximally from the circulatory support device such that visibility of the visual marker on a proximal side of the hub indicates the circulatory support device is located within the tubular body of the insertion catheter.

Example 18: The insertion catheter of any example herein, wherein the circulatory support device comprises a tubular housing, a motor, an impeller configured to be rotated by the motor, a first guidewire port on a distal end of the tubular housing, a second guidewire port on a sidewall of the tubular housing distal to the impeller, and a third guidewire port on a proximal side of the impeller, and wherein the distal end of the tubular body of the insertion catheter detachably connects to a guidewire aid configured to facilitate entry of a guidewire through the first guidewire port.

Example 19: The insertion catheter of any example herein, in particular Example 18, wherein the tubular body of the insertion catheter is configured to receive the circulatory support device with a removable guidewire guide tube, wherein the removable guidewire guide tube enters the first guidewire port on the distal end of the tubular housing, exits the tubular housing via the second guidewire port on the sidewall of the tubular housing distal to the impeller, reenters the tubular housing via the third guidewire port on the proximal side of the impeller, and extends proximally into the catheter shaft.

Example 20: The insertion catheter of any example herein, in particular Examples 2 to 19, wherein the hub comprises one or more features for preventing axial and optionally rotational movement of the circulatory support catheter.

Example 21: The insertion catheter of any example herein, in particular Examples 2 to 20, wherein the hub of the insertion catheter comprises a locking mechanism, the locking mechanism comprising a recess configured to accept a locking pad configured to releasably lock with the circulatory support catheter.

Example 22: The insertion catheter of any example herein, in particular Example 21, wherein the hub of the insertion catheter comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the circulatory support catheter.

Example 23: The insertion catheter of any example herein, in particular Example 22, wherein inward compression of the tabs of the locking mechanism compresses the locking pad against the circulatory support catheter.

Example 24: The insertion catheter of any example herein, in particular Examples 21 to 23, wherein the locking pad is configured to releasably lock with the catheter shaft of the circulatory support catheter.

Example 25: The insertion catheter of any example herein, wherein the tubular body of the insertion catheter has a length within a range of from about 275 mm to about 675 mm and an inside diameter within a range of from about 1.5 mm to about 6 mm.

Example 26: The insertion catheter of any example herein, wherein the distal portion of the tubular body has a length within a range of from about 75 mm to about 140 mm and an inside diameter within a range of from about 3.5 mm to about 6 mm, wherein the proximal portion of the tubular body has a length within a range of from about 100 mm to about 165 mm and an inside diameter within a range of from about 2.0 mm to about 4.5 mm, and wherein the mid portion of the tubular body has a length within a range of from about 150 mm to about 250 mm and an inside diameter within a range of from about 1.5 mm to about 4.5 mm.

Example 27: The insertion catheter of any example herein, wherein the tubular portion comprises a braided wire.

Example 28: The insertion catheter of any example herein, in particular Example 27, wherein the distal end of the tubular body comprises a PET band configured to hold ends of the braided wire.

Example 29: The insertion catheter of any example herein, wherein the distal end of the tubular body comprises a radio opaque marker.

Example 30: The insertion catheter of any example herein, wherein the proximal portion of the tubular body is configured to transmit longitudinal force without kinking.

Example 31: The insertion catheter of any example herein, wherein the proximal portion of the tubular body is more stiff than the mid portion and the distal portion.

Example 32: The insertion catheter of any example herein, wherein the proximal portion of the tubular body comprises a reinforced double-braided wire.

Example 33: The insertion catheter of any example herein, wherein at least the distal portion of the tubular body is coated with a hydrophilic coating configured to reduce friction on an exterior surface thereof.

Example 34: A mechanical circulatory support system, comprising: a circulatory support catheter, comprising a circulatory support device carried by an elongate flexible catheter shaft, the circulatory support device comprising a tubular housing, a motor, and an impeller configured to be rotated by the motor; and an insertion catheter comprising a tubular body having a proximal end, a distal end, a longitudinal axis extending between the proximal and distal ends, a proximal portion adjacent the proximal end, a distal portion adjacent the distal end, and a mid portion between the proximal portion and the distal portion, wherein the distal portion of the tubular body is configured to axially movably receive the circulatory support device, and wherein a diameter of the distal portion is greater than a diameter of the mid portion.

Example 35: The insertion catheter of any example herein, in particular Example 34, wherein the impeller is configured to be rotated by the motor via a shaft.

Example 36: The insertion catheter of any example herein, in particular Example 34, wherein the impeller is configured to be rotated by the motor via a magnetic coupling.

Example 37: The insertion catheter of any example herein, in particular Examples 34 to 36, wherein the system does not require purging.

Example 38: The insertion catheter of any example herein, in particular Examples 34 to 37, wherein the insertion catheter further comprises a hub having a proximal end and a distal end, wherein the distal end of the hub fluidly connects with the proximal end of the tubular body.

Example 39: The insertion catheter of any example herein, in particular Examples 34 to 38, wherein the tubular body further comprises a proximal transition section between the proximal portion and the mid portion and a distal transition section between the distal portion and the mid portion.

Example 40: The insertion catheter of any example herein, in particular Examples 34 to 39, wherein the distal portion and the distal transition section are configured to receive the circulatory support device and the mid portion, proximal transition section, proximal portion, and hub are configured to receive the elongate flexible catheter shaft.

Example 41: The insertion catheter of any example herein, in particular Examples 34 to 40, wherein the distal portion, mid portion, and proximal portion are concentric about the longitudinal axis.

Example 42: The insertion catheter of any example herein, in particular Examples 34 to 41, wherein the mid portion comprises a smaller outer diameter than an outer diameter of the proximal portion and an outer diameter of the distal portion.

Example 43: The insertion catheter of any example herein, in particular Examples 34 to 42, wherein the distal portion comprises a larger inner diameter than an inner diameter of the mid portion and the proximal portion.

Example 44: The insertion catheter of any example herein, in particular Examples 34 to 43, wherein the tubular body is configured so that the proximal portion remains outside the body of a patient when the insertion catheter is in use.

Example 45: The insertion catheter of any example herein, in particular Examples 34 to 44, wherein the tubular body is configured so that the mid portion extends from outside the body of a patient through an arteriotomy of a femoral artery, through the femoral artery, through a bifurcation of the femoral artery where it joins an aorta, and into the aorta when the insertion catheter is in use.

Example 46: The insertion catheter of any example herein, in particular Examples 34 to 45, wherein the tubular body is configured so that the distal portion is positioned within an aorta of a patient when the insertion catheter is in use.

Example 47: The insertion catheter of any example herein, in particular Examples 34 to 46, wherein the distal end of the tubular body is configured to extend past a distal end of an introducer sheath when the insertion catheter is fully docked with the introducer sheath when in use.

Example 48: The insertion catheter of any example herein, in particular Example 47, wherein the distal end of the hub comprises one or more features for preventing rotational and/or axial movement of the insertion catheter when docked with the introducer sheath.

Example 49: The insertion catheter of any example herein, in particular Examples 34 to 48, wherein the insertion catheter comprises a tube with a valve in fluid communication with an inner lumen of the tubular body of the insertion catheter configured for flushing with saline.

Example 50: The insertion catheter of any example herein, in particular Examples 34 to 49, wherein the insertion catheter comprises a hemostatic valve.

Example 51: The insertion catheter of any example herein, in particular Examples 34 to 50, wherein the insertion catheter comprises a plug disposed at the proximal end of the hub configured to connect to a sterile shield sleeve.

Example 52: The insertion catheter of any example herein, in particular Examples 34 to 51, wherein the elongate flexible catheter shaft comprises a visual marker spaced proximally from the circulatory support device such that visibility of the visual marker on a proximal side of the hub indicates the circulatory support device is located within the tubular body of the insertion catheter.

Example 53: The insertion catheter of any example herein, in particular Examples 34 to 52, wherein the circulatory support device comprises a tubular housing, a motor, an impeller configured to be rotated by the motor, a first guidewire port on a distal end of the tubular housing, a second guidewire port on a sidewall of the tubular housing distal to the impeller, and a third guidewire port on a proximal side of the impeller, and wherein the distal end of the tubular body of the insertion catheter detachably connects to a guidewire aid configured to facilitate entry of a guidewire through the first guidewire port.

Example 54: The insertion catheter of any example herein, in particular Example 53, wherein the tubular body of the insertion catheter is configured to receive the circulatory support device with a removable guidewire guide tube, wherein the removable guidewire guide tube enters the first guidewire port on the distal end of the tubular housing, exits the tubular housing via the second guidewire port on the sidewall of the tubular housing distal to the impeller, reenters the tubular housing via the third guidewire port on the proximal side of the impeller, and extends proximally into the catheter shaft.

Example 55: The insertion catheter of any example herein, in particular Examples 38 to 54, wherein the hub comprises one or more features for preventing axial and optionally rotational movement of the circulatory support catheter.

Example 56: The insertion catheter of any example herein, in particular Examples 38 to 55, wherein the hub of the insertion catheter comprises a locking mechanism, the locking mechanism comprising a recess configured to accept a locking pad configured to releasably lock with the circulatory support catheter.

Example 57: The insertion catheter of any example herein, in particular Example 56, wherein the hub of the insertion catheter comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the circulatory support catheter.

Example 58: The insertion catheter of any example herein, in particular Example 57, wherein inward compression of the tabs of the locking mechanism compresses the locking pad against the circulatory support catheter.

Example 59: The insertion catheter of any example herein, in particular Examples 56 to 58, wherein the locking pad is configured to releasably lock with the catheter shaft of the circulatory support catheter.

Example 60: The insertion catheter of any example herein, in particular Examples 34 to 59, wherein the tubular body of the insertion catheter has a length within a range of from about 275 mm to about 675 mm and an inside diameter within a range of from about 1.5 mm to about 6 mm.

Example 61: The insertion catheter of any example herein, in particular Examples 34 to 60, wherein the distal portion of the tubular body has a length within a range of from about 75 mm to about 140 mm and an inside diameter within a range of from about 3.5 mm to about 6 mm, wherein the proximal portion of the tubular body has a length within a range of from about 100 mm to about 165 mm and an inside diameter within a range of from about 2.0 mm to about 4.5 mm, and wherein the mid portion of the tubular body has a length within a range of from about 150 mm to about 250 mm and an inside diameter within a range of from about 1.5 mm to about 4.5 mm.

Example 62: The insertion catheter of any example herein, in particular Examples 34 to 61, wherein the tubular portion comprises a braided wire.

Example 63: The insertion catheter of any example herein, in particular Examples 34 to 62, wherein the distal end of the tubular body comprises a PET band configured to hold ends of the braided wire.

Example 64: The insertion catheter of any example herein, in particular Examples 34 to 63, wherein the distal end of the tubular body comprises a radio opaque marker.

Example 65: The insertion catheter of any example herein, in particular Examples 34 to 64, wherein the proximal portion of the tubular body is configured to transmit longitudinal force without kinking.

Example 66: The insertion catheter of any example herein, in particular Examples 34 to 65, wherein the proximal portion of the tubular body is more stiff than the mid portion and the distal portion.

Example 67: The insertion catheter of any example herein, in particular Examples 34 to 66, wherein the proximal portion of the tubular body comprises a reinforced double-braided wire.

Example 68: The insertion catheter of any example herein, in particular Examples 34 to 67, wherein at least the distal portion of the tubular body is coated with a hydrophilic coating configured to reduce friction on an exterior surface thereof.

Example 69: A method of using an insertion catheter with a circulatory support catheter having a circulatory support device carried by an elongate flexible catheter shaft, the method comprising: inserting a distal end of the insertion catheter through a proximal end of an introducer sheath that has been advanced into an artery of a patient, and advancing the insertion catheter through the introducer sheath and any hemostatic valves of the introducer sheath until the distal end of the insertion catheter extends past a distal end of the introducer sheath, wherein the insertion catheter comprises a tubular body having a distal portion adjacent the distal end configured to axially movably receive the circulatory support device, and wherein the insertion catheter is configured to protect the circulatory support device as it is advanced through the introducer sheath and any hemostatic valves of the introducer sheath.

Example 70: The method of using an insertion catheter of any example herein, in particular Example 69, wherein the insertion catheter further comprises a hub fluidically connected to a proximal end of the tubular body, the hub configured to lock with the introducer sheath and prevent axial and/or rotational movement of the insertion catheter relative to the introducer sheath when the insertion catheter is fully advanced through the introducer sheath.

Example 71: The method of using an insertion catheter of any example herein, in particular Examples 69 to 70, wherein the tubular body further comprises a proximal portion adjacent its proximal end and a mid portion between the proximal portion and the distal portion.

Example 72: The method of using an insertion catheter of any example herein, in particular Examples 71, wherein the tubular body further comprises a proximal transition section between the proximal portion and the mid portion and a distal transition section between the distal portion and the mid portion.

Example 73: The method of using an insertion catheter of any example herein, in particular Example 72, wherein the distal portion and the distal transition section are configured to receive the circulatory support device and the mid portion, proximal transition section, proximal portion, and hub are configured to receive the elongate flexible catheter shaft.

Example 74: The method of using an insertion catheter of any example herein, in particular Examples 71 to 73, wherein the tubular body is configured so that the proximal portion remains outside the body of a patient when the insertion catheter is fully advanced through the introducer sheath.

Example 75: The method of using an insertion catheter of any example herein, in particular Examples 69 to 74, wherein the tubular body is configured so that the distal portion is positioned within an aorta of a patient when the insertion catheter is fully advanced through the introducer sheath.

Example 76: The method of using an insertion catheter of any example herein, in particular Examples 71 to 75, wherein the tubular body is configured so that the mid portion extends from outside the body of a patient through an arteriotomy of a femoral artery, through the femoral artery, through a bifurcation of the femoral artery where it joins an aorta, and into the aorta when the insertion catheter is fully advanced through the introducer sheath.

Example 77: The method of using an insertion catheter of any example herein, in particular Examples 69 to 76, further comprising advancing the circulatory support catheter through the insertion catheter until its target therapeutic location is reached within a patient.

Example 78: The method of using an insertion catheter of any example herein, in particular Example 77, wherein the hub of the insertion catheter comprises a locking mechanism, and the method further comprises locking the axial location of the circulatory support catheter relative to the insertion catheter.

Example 79: The method of using an insertion catheter of any example herein, in particular Example 78, wherein the locking mechanism comprising a recess configured to accept a locking pad configured to releasably lock with the circulatory support catheter.

Example 80: The method of using an insertion catheter of any example herein, in particular Examples 78 to 79, wherein the hub of the insertion catheter comprises a housing surrounding at least a portion of the locking mechanism, the housing comprising opposing first inner surface walls spaced farther than opposing second inner surface walls, wherein the at least a portion of the locking mechanism comprises radially outwardly extending tabs, and wherein the housing is configured to rotate to inwardly compress the tabs to prevent axial movement of the circulatory support catheter.

Example 81: The method of using an insertion catheter of any example herein, in particular Example 80, wherein inward compression of the tabs of the locking mechanism compresses the locking pad against the circulatory support catheter.

Example 82: The method of using an insertion catheter of any example herein, in particular Examples 79 to 81, wherein the locking pad is configured to releasably lock with the catheter shaft of the circulatory support catheter.

Example 83: The method of using an insertion catheter of any example herein, in particular Examples 79 to 82, further comprising rotating the housing of the hub to releasably lock the axial location of the circulatory support catheter relative to the insertion catheter.

Example 84: A medical delivery system comprising an insertion tool comprising an insertion tool hub and an insertion tool shaft extending distally from the insertion tool hub, and a peel-away catheter comprising a catheter shaft configured to be separable into a plurality of elongate shaft portions, and the catheter shaft comprising a proximal portion configured to mate with a distal portion of the insertion tool shaft.

Example 85: The system of any example herein, in particular example 84, wherein the proximal portion of the catheter shaft comprises an insertion tool mating portion configured to be disposed over and have an interference fit with an exterior surface of the distal portion of the insertion tool shaft.

Example 86: The system of any example herein, in particular example 85, wherein the insertion tool mating portion comprises a flared configuration.

Example 87: The system of any example herein, in particular examples 84 to 86, wherein the peel-away catheter further comprises a first and second operator engagement handle coupled to respective portions of the proximal portion of the catheter shaft for engagement by an operator to separate the catheter shaft into the plurality of elongate shaft portions.

Example 88: The system of any example herein, in particular examples 84 to 86, wherein the peel-away catheter and the insertion tool are configured to slidably receive respective portions of a Mechanical Circulatory Support (MCS) system.

Example 89: The system of any example herein, in particular example 88, wherein corresponding portions of the Mechanical Circulatory Support (MCS) system are configured to be disposed through respective delivery lumens of the insertion tool and peel-away catheter, a distal tip of the Mechanical Circulatory Support (MCS) system being configured to be disposed distally of the catheter shaft.

Example 90: The system of any example herein, in particular example 88, further comprising a support sleeve comprising slit and configured to be disposed around a portion of a shaft of the Mechanical Circulatory Support (MCS) system, a corresponding portion of a delivery lumen of the catheter shaft being configured to slidably receive the support sleeve and the portion of the shaft of the Mechanical Circulatory Support (MCS) system.

Example 91: The system of any example herein, in particular examples 84-86, further comprising an introducer sheath comprising an introducer sheath shaft extending distally from an introducer sheath hub, and wherein at least a portion of the catheter shaft is configured to be slidably disposed within the introducer sheath shaft and the introducer sheath hub while the proximal portion of the catheter shaft is mated with the distal portion of the insertion tool shaft.

Example 92: The system of any example herein, in particular example 91, further comprising a spacer configured to engage with a spacer engagement feature of the introducer sheath hub and a spacer engagement feature of the insertion tool hub to axially space and align the introducer sheath hub and the insertion tool hub.

Example 93: The system of any example herein, in particular example 92, wherein the spacer comprises a distal portion configured to engage with the spacer engagement feature of the introducer sheath hub, a proximal portion configured to engage with the spacer engagement feature of the insertion tool hub, and a medial portion extending between and perpendicular to the distal and proximal portions, the distal portion and proximal portion each comprising a concave edge configured to mate with a respective groove of the spacer engagement feature of the introducer sheath hub or insertion tool hub.

Example 94: A medical delivery system comprising an introducer sheath including an introducer sheath shaft extending distally from an introducer sheath hub, a peel-away catheter including a catheter shaft extending distally from a catheter hub, the introducer sheath and peel-away catheter being configured to slidably receive a Mechanical Circulatory Support (MCS) system. The system can include a spacer configured to engage with the introducer sheath hub and the catheter hub to axial space the introducer sheath hub from the catheter hub.

Example 95: The system of any example herein, in particular example 94, wherein the peel-away catheter comprises a first elongate member comprising a first elongate portion having at least a portion configured to be slidably disposed in a first elongate member lumen extending along a first longitudinal portion of a shaft wall of the catheter shaft while the peel-away catheter is in an unpeeled configuration. The peel-away catheter can include a second elongate member comprising a second elongate portion having at least a portion configured to be slidably disposed in a second elongate member lumen extending along a second longitudinal portion of the shaft wall of the catheter shaft while the peel-away catheter is in the unpeeled configuration, the first and second elongate portions being configured to be pulled laterally to cut through corresponding shaft wall portions between the first and second elongate member lumens and an exterior surface of the shaft wall for separating the shaft wall into first and second elongate shaft wall portions.

Example 96: The system of any example herein, in particular example 95, wherein the first elongate portion is opposingly disposed around a circumference of the catheter shaft relative to the second elongate portion.

Example 97: The system of any example herein, in particular example 94 or 95, wherein the peel-away catheter comprises a first proximal shaft tab coupled to a proximal portion of the first elongate shaft wall portion, and a second proximal shaft tab coupled to a proximal portion of the second longitudinal shaft wall portion, the first and second proximal shaft tabs being configured to be pulled laterally to tear corresponding shaft wall portions between the first and second elongate member lumens and an interior surface of the shaft wall for separating the shaft wall into the first and second elongate shaft wall portions.

Example 98: The system of any example herein, in particular example 97, wherein the first and second proximal shaft tabs are disposed at opposing positions around a circumference of the catheter shaft.

Example 99: The system of any example herein, in particular example 94, wherein the peel-away catheter comprises a first elongate member comprising a first elongate portion, a second elongate member comprising a second elongate portion, and a first and second elongate wall portions. While the catheter shaft is in an unpeeled configuration, the first elongate portion can be between a first edge of the second elongate shaft wall portion and a second edge of the first elongate shaft wall portion, and the second elongate portion can be between a first edge of the first elongate shaft wall portion and a second edge of the second elongate shaft wall portion, the first and second elongate portions being configured to be pulled laterally to separate the first and second elongate portions from the first and second elongate shaft wall portions.

Example 100: The system of any example herein, in particular example 99, wherein the first elongate portion is opposingly disposed around a circumference of the catheter shaft relative to the second elongate portion, and the first elongate shaft wall portion is opposingly disposed around the circumference of the catheter shaft relative to the second elongate shaft wall portion.

Example 101: The system of any example herein, in particular example 94, wherein the peel-away catheter comprises an elongate member comprising an elongate portion, and wherein a shaft wall of the catheter shaft comprises a first interlocking portion comprising a first plurality of mating portions along a longitudinal portion of the catheter shaft, and a second interlocking portion comprising a second plurality of mating portions along the longitudinal portion of the catheter shaft and configured to mate with the first interlocking portion. The peel-away catheter can include an elongate member comprising an elongate portion, corresponding portions of the elongate portion being configured to be slidably disposed within respective elongate member lumen portions of each of the first and second plurality of mating portions of the first and second interlocking portions.

Example 102: The system of any example herein, in particular example 101, wherein mating edges of the first and second interlocking portions have a rectangular wave shape.

Example 103: The system of any example herein, in particular example 101 or 102, wherein the elongate portion is configured to be withdrawn from the elongate member lumen portions to allow the first and second interlocking portions to separate.

Example 104: The system of any example herein, in particular example 94, wherein the peel-away catheter comprises an elongate member configured to fasten together edges of a shaft wall of the catheter shaft to maintain the catheter shaft in an unpeeled configuration.

Example 105: The system of any example herein, in particular example 104, wherein the shaft wall comprises a first edge portion comprises a plurality of first openings at respective locations along a longitudinal portion of the first edge portion, and a second edge portion comprising a plurality of second openings at respective locations along a longitudinal portion of the second edge portion. The elongate member can include an elongate portion, corresponding portions of the elongate portion being configured to be disposed through the plurality of first and second openings in alternating fashion to fasten together the first edge portion and the second edge portion together.

Example 106: The system of any example herein, in particular example 104 or 105, wherein the elongate portion is configured to be withdrawn from the plurality of first and second openings to allow the first and second edge portions to separate.

Example 107: The system of any example herein, in particular example 94, wherein the catheter shaft is configured to be in a rolled configuration while the catheter shaft is in the unpeeled configuration.

Example 108: The system of any example herein, in particular example 107, wherein a shaft wall of the catheter shaft comprises a first overlapping wall portion, and a second overlapping wall portion, and while the catheter shaft is in the rolled configuration, the first overlapping wall portion being configured to be over and in contact with the second overlapping wall portion along a longitudinal portion of the shaft wall.

Example 109: The system of any example herein, in particular example 107 or 108, wherein the peel-away catheter comprises a proximal shaft tab coupled to a proximal portion of a shaft wall of the catheter shaft, the proximal shaft tab is coupled at a position circumferentially spaced from the first and second overlapping wall portions.

Example 110: The system of any example herein, in particular example 109, wherein the proximal shaft tab is opposingly oriented around a circumference of the catheter shaft relative to the first and second overlapping wall portions.

Example 111: The system of any example herein, in particular examples 94 to 110, wherein at least one of an outer diameter of the catheter shaft and a diameter of the shaft delivery lumen of the catheter shaft is configured to conform to that of corresponding portions of the MCS system disposed therethrough.

Example 112: The system of any example herein, in particular examples 94 to 111, wherein the spacer is configured to engage with a spacer engagement feature of the introducer sheath hub and a spacer engagement feature of the catheter hub to axially space and align the introducer sheath hub and the catheter hub.

Example 113: The system of any example herein, in particular example 112, wherein the spacer comprises a distal portion configured to engage with the spacer engagement feature of the introducer sheath hub, a proximal portion configured to engage with the spacer engagement feature of the catheter hub, and a medial portion extending between and perpendicular to the distal and proximal portions, the distal portion and proximal portion each comprising a concave edge configured to mate with a respective groove of the spacer engagement feature of the introducer sheath hub or catheter hub.

Example 114: The system of any example herein, in particular examples 94 to 113, wherein the catheter hub is not a peel-away hub.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

If an exemplary embodiment comprises a “and/or” link between a first feature and a second feature, this is to be read in such a way that the embodiment according to one embodiment has both the first feature and the second feature and according to a further embodiment has either only the first feature or only the second feature.

What is claimed is:

1. An insertion catheter for a circulatory support catheter having a circulatory support device carried by an elongate flexible catheter shaft, the insertion catheter comprising:
a tubular body having a proximal end, a distal end, a longitudinal axis extending between the proximal end and the distal end, a proximal portion adjacent the proximal end, a distal portion adjacent the distal end, and a middle portion between the proximal portion and the distal portion;
wherein the distal portion of the tubular body is configured to axially movably receive the circulatory support device; and
wherein diameters of the distal portion and the proximal portion are greater than a diameter of the middle portion.

2. The insertion catheter of claim 1, further comprising a hub having a proximal end and a distal end, wherein the distal end of the hub fluidly connects with the proximal end of the tubular body.

3. The insertion catheter of claim 1, wherein the tubular body further comprises a proximal transition section between the proximal portion and the middle portion and a distal transition section between the distal portion and the middle portion, wherein the distal portion and the distal transition section are configured to receive the circulatory support device and the middle portion, proximal transition section, proximal portion, and a hub are configured to receive the elongate flexible catheter shaft.

4. The insertion catheter of claim 1, wherein the middle portion comprises a smaller outer diameter than an outer diameter of the proximal portion and an outer diameter of the distal portion.

5. The insertion catheter of claim 1, wherein the distal portion comprises a larger inner diameter than an inner diameter of the middle portion and the proximal portion.

6. The insertion catheter of claim 1, wherein the distal end of the tubular body is configured to extend past a distal end of an introducer sheath when the insertion catheter is fully docked with the introducer sheath in use.

7. The insertion catheter of claim 6, wherein a distal end of a hub comprises one or more features for preventing rotational and/or axial movement of the insertion catheter when docked with the introducer sheath.

8. The insertion catheter of claim 1, wherein the circulatory support device comprises a tubular housing, a motor, and an impeller configured to be rotated by the motor.

9. The insertion catheter of claim 1, wherein the proximal portion of the tubular body is stiffer than the middle portion and the distal portion.

10. A mechanical circulatory support system, comprising:
a circulatory support catheter, comprising a circulatory support device carried by an elongate flexible catheter shaft, the circulatory support device comprising a tubular housing, a motor, and an impeller configured to be rotated by the motor; and
an insertion catheter comprising a tubular body having a proximal end, a distal end, a longitudinal axis extending between the proximal end and the distal end, a proximal portion adjacent the proximal end, a distal portion adjacent the distal end, and a middle portion between the proximal portion and the distal portion;
wherein the distal portion of the tubular body is configured to axially movably receive the circulatory support device; and
wherein diameters of the distal portion and the proximal portion are greater than a diameter of the middle portion.

11. The system of claim 10, wherein the impeller is configured to be rotated by the motor via a shaft or a magnetic coupling.

12. The system of claim 10, wherein the system does not require purging.

13. The system of claim 10, wherein the middle portion comprises a smaller outer diameter than an outer diameter of the proximal portion and an outer diameter of the distal portion.

14. The system of claim 10, wherein the distal portion comprises a larger inner diameter than an inner diameter of the middle portion and the proximal portion.

15. The system of claim 10, wherein the insertion catheter comprises a hemostatic valve.

16. A method of using an insertion catheter with a circulatory support catheter having a circulatory support device carried by an elongate flexible catheter shaft, the method comprising:
inserting a distal end of the insertion catheter through a proximal end of an introducer sheath that has been advanced into an artery of a patient; and
advancing the insertion catheter through the introducer sheath until the distal end of the insertion catheter extends past a distal end of the introducer sheath;
wherein the insertion catheter comprises a tubular body having a distal portion adjacent the distal end configured to axially movably receive the circulatory support device; and
wherein the insertion catheter is configured to protect the circulatory support device as it is advanced through the introducer sheath.

17. The method of claim 16, wherein the insertion catheter further comprises a hub fluidically connected to a proximal end of the tubular body, the hub configured to lock with the introducer sheath and prevent axial and/or rotational movement of the insertion catheter relative to the introducer sheath when the insertion catheter is fully advanced through the introducer sheath.

18. The method of claim 16, wherein the tubular body is configured so that the proximal end remains outside a body of a patient when the insertion catheter is fully advanced through the introducer sheath.

19. The method of claim 16, wherein the tubular body is configured so that the distal portion is positioned within an aorta of a patient when the insertion catheter is fully advanced through the introducer sheath.

20. The method of claim 16, wherein the tubular body is configured so that at least a portion of the tubular body extends from outside a body of a patient through an arteriotomy of a femoral artery, through the femoral artery, through a bifurcation of the femoral artery where it joins an aorta, and into the aorta, when the insertion catheter is fully advanced through the introducer sheath.
